SEQUENCE LISTING (288 sequences)

SEQ ID NO: 1 - RNA, 11940 nt, Homo sapiens
  source 1..11940: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaa

SEQ ID NO: 2 - RNA, 13079 nt, Homo sapiens
  source 1..13079: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 3 - RNA, 12941 nt, Homo sapiens
  source 1..12941: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 4 - RNA, 12890 nt, Homo sapiens
  source 1..12890: mol_type = genomic RNA
aagtgtaggagacacactgctggcctgtggaaactcatggaactgttcctccagattaacacttcaggggttatggaagctggaggaagctgagcttttactacatcttttgggggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 5 - RNA, 12933 nt, Homo sapiens
  source 1..12933: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 6 - RNA, 12765 nt, Homo sapiens
  source 1..12765: mol_type = genomic RNA
aagtgtaggagacacactgctggcctgtggaaactcatggaactgttcctccagattaacacttcaggggttatggaagctggaggaagctgagcttttactacatcttttgggggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 7 - RNA, 12857 nt, Homo sapiens
  source 1..12857: mol_type = genomic RNA
aagtgtaggagacacactgctggcctgtggaaactcatggaactgttcctccagattaacacttcaggggttatggaagctggaggaagctgagcttttactacatcttttgggggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 8 - RNA, 12900 nt, Homo sapiens
  source 1..12900: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 9 - RNA, 12992 nt, Homo sapiens
  source 1..12992: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 10 - RNA, 12989 nt, Homo sapiens
  source 1..12989: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 11 - RNA, 12897 nt, Homo sapiens
  source 1..12897: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 12 - RNA, 12849 nt, Homo sapiens
  source 1..12849: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 13 - RNA, 12806 nt, Homo sapiens
  source 1..12806: mol_type = genomic RNA
aagtgtaggagacacactgctggcctgtggaaactcatggaactgttcctccagattaacacttcaggggttatggaagctggaggaagctgagcttttactacatcttttgggggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 14 - RNA, 12938 nt, Homo sapiens
  source 1..12938: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 15 - RNA, 13008 nt, Homo sapiens
  source 1..13008: mol_type = genomic RNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggtaagcatattggaaggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 16 - RNA, 26 nt, synthetic construct
  source 1..26: mol_type = other RNA
aataaagggctcaggggaggaaccag

SEQ ID NO: 17 - RNA, 26 nt, synthetic construct
  source 1..26: mol_type = other RNA
aaagggctcaggggaggaaccagcgc

SEQ ID NO: 18 - RNA, 26 nt, synthetic construct
  source 1..26: mol_type = other RNA
gggctcaggggaggaaccagcgctcc

SEQ ID NO: 19 - RNA, 26 nt, synthetic construct
  source 1..26: mol_type = other RNA
tcaggggaggaaccagcgctccaccc

SEQ ID NO: 20 - RNA, 26 nt, synthetic construct
  source 1..26: mol_type = other RNA
ccataataaagggctcaggggaggaa

SEQ ID NO: 21 - RNA, 27 nt, synthetic construct
  source 1..27: mol_type = other RNA
ccaagttggagcaagattatcctatac

SEQ ID NO: 22 - RNA, 30 nt, synthetic construct
  source 1..30: mol_type = other RNA
gtacccataataaagggctcaggggaggaa

SEQ ID NO: 23 - RNA, 26 nt, synthetic construct
  source 1..26: mol_type = other RNA
gtacccataataaagggctcagggga

SEQ ID NO: 24 - RNA, 27 nt, synthetic construct
  source 1..27: mol_type = other RNA
ggtagcaaaaggggtaatacagtaccc

SEQ ID NO: 25 - RNA, 28 nt, synthetic construct
  source 1..28: mol_type = other RNA
ccccatccaagttggagcaagattatcc

SEQ ID NO: 26 - RNA, 20 nt, synthetic construct
  source 1..20: mol_type = other RNA
gttggagcaagattatccta

SEQ ID NO: 27 - RNA, 20 nt, synthetic construct
  source 1..20: mol_type = other RNA
aaaggggtaatacagtaccc

SEQ ID NO: 28 - RNA, 20 nt, synthetic construct
  source 1..20: mol_type = other RNA
cccataataaagggctcagg

SEQ ID NO: 29 - RNA, 20 nt, synthetic construct
  source 1..20: mol_type = other RNA
aataaagggctcaggggagg

SEQ ID NO: 30 - RNA, 18 nt, synthetic construct
  source 1..18: mol_type = other RNA
aggggtaatacagtaccc

SEQ ID NO: 31 - RNA, 18 nt, synthetic construct
  source 1..18: mol_type = other RNA
aaggattaaaggtagcaa

SEQ ID NO: 32 - RNA, 18 nt, synthetic construct
  source 1..18: mol_type = other RNA
cataagtcacagtgcaag

SEQ ID NO: 33 - RNA, 26 nt, synthetic construct
  source 1..26: mol_type = other RNA
ccactacacataagtcacagtgcaag

SEQ ID NO: 34 - RNA, 26 nt, synthetic construct
  source 1..26: mol_type = other RNA
tacacataagtcacagtgcaaggatt

SEQ ID NO: 35 - RNA, 26 nt, synthetic construct
  source 1..26: mol_type = other RNA
cataagtcacagtgcaaggattaaag

SEQ ID NO: 36 - RNA, 26 nt, synthetic construct
  source 1..26: mol_type = other RNA
agtcacagtgcaaggattaaaggtag

SEQ ID NO: 37 - RNA, 26 nt, synthetic construct
  source 1..26: mol_type = other RNA
acagtgcaaggattaaaggtagcaaa

SEQ ID NO: 38 - RNA, 26 nt, synthetic construct
  source 1..26: mol_type = other RNA
tgcaaggattaaaggtagcaaaaggg

SEQ ID NO: 39 - RNA, 26 nt, synthetic construct
  source 1..26: mol_type = other RNA
aggattaaaggtagcaaaaggggtaa

SEQ ID NO: 40 - RNA, 26 nt, synthetic construct
  source 1..26: mol_type = other RNA
ttaaaggtagcaaaaggggtaataca

SEQ ID NO: 41 - RNA, 26 nt, synthetic construct
  source 1..26: mol_type = other RNA
aggtagcaaaaggggtaatacagtac

SEQ ID NO: 42 - RNA, 26 nt, synthetic construct
  source 1..26: mol_type = other RNA
agcaaaaggggtaatacagtacccat

SEQ ID NO: 43 - RNA, 26 nt, synthetic construct
  source 1..26: mol_type = other RNA
aaaggggtaatacagtacccataata

SEQ ID NO: 44 - RNA, 26 nt, synthetic construct
  source 1..26: mol_type = other RNA
gggtaatacagtacccataataaagg

SEQ ID NO: 45 - RNA, 26 nt, synthetic construct
  source 1..26: mol_type = other RNA
gctccaccccatccaagttggagcaa

SEQ ID NO: 46 - RNA, 26 nt, synthetic construct
  source 1..26: mol_type = other RNA
ccagcgctccaccccatccaagttgg

SEQ ID NO: 47 - RNA, 26 nt, synthetic construct
  source 1..26: mol_type = other RNA
aggaaccagcgctccaccccatccaa

SEQ ID NO: 48 - RNA, 21 nt, synthetic construct
  source 1..21: mol_type = other RNA
agttggagcaagattatccta

SEQ ID NO: 49 - RNA, 21 nt, synthetic construct
  source 1..21: mol_type = other RNA
ccaagttggagcaagattatc

SEQ ID NO: 50 - RNA, 18 nt, synthetic construct
  source 1..18: mol_type = other RNA
agttggagcaagattatc

SEQ ID NO: 51 - RNA, 18 nt, synthetic construct
  source 1..18: mol_type = other RNA
agtacccataataaaggg

SEQ ID NO: 52 - RNA, 40 nt, synthetic construct
  source 1..40: mol_type = other RNA
aaaggggtaatacagtacccgttggagcaagattatccta

SEQ ID NO: 53 - RNA, 38 nt, synthetic construct
  source 1..38: mol_type = other RNA
aggggtaatacagtacccgttggagcaagattatccta

SEQ ID NO: 54 - RNA, 47 nt, synthetic construct
  source 1..47: mol_type = other RNA
aaaggggtaatacagtacccccaagttggagcaagattatcctatac

SEQ ID NO: 55 - RNA, 45 nt, synthetic construct
  source 1..45: mol_type = other RNA
aggggtaatacagtacccccaagttggagcaagattatcctatac

SEQ ID NO: 56 - RNA, 40 nt, synthetic construct
  source 1..40: mol_type = other RNA
gttggagcaagattatcctaaaaggggtaatacagtaccc

SEQ ID NO: 57 - RNA, 38 nt, synthetic construct
  source 1..38: mol_type = other RNA
gttggagcaagattatcctaaggggtaatacagtaccc

SEQ ID NO: 58 - RNA, 47 nt, synthetic construct
  source 1..47: mol_type = other RNA
ccaagttggagcaagattatcctatacaaaggggtaatacagtaccc

SEQ ID NO: 59 - RNA, 45 nt, synthetic construct
  source 1..45: mol_type = other RNA
ccaagttggagcaagattatcctatacaggggtaatacagtaccc

SEQ ID NO: 60 - RNA, 53 nt, synthetic construct
  source 1..53: mol_type = other RNA
ccaagttggagcaagattatcctatacacagtgcaaggattaaaggtagcaaa

SEQ ID NO: 61 - RNA, 53 nt, synthetic construct
  source 1..53: mol_type = other RNA
acagtgcaaggattaaaggtagcaaaccaagttggagcaagattatcctatac

SEQ ID NO: 62 - RNA, 53 nt, synthetic construct
  source 1..53: mol_type = other RNA
ccaagttggagcaagattatcctatacaggattaaaggtagcaaaaggggtaa

SEQ ID NO: 63 - RNA, 53 nt, synthetic construct
  source 1..53: mol_type = other RNA
aggattaaaggtagcaaaaggggtaaccaagttggagcaagattatcctatac

SEQ ID NO: 64 - RNA, 45 nt, synthetic construct
  source 1..45: mol_type = other RNA
ccaagttggagcaagattatcctatacagtacccataataaaggg

SEQ ID NO: 65 - RNA, 45 nt, synthetic construct
  source 1..45: mol_type = other RNA
agtacccataataaagggccaagttggagcaagattatcctatac

SEQ ID NO: 66 - RNA, 44 nt, synthetic construct
  source 1..44: mol_type = other RNA
aggaaccagcgctccaccccatccaaaggggtaatacagtaccc

SEQ ID NO: 67 - RNA, 44 nt, synthetic construct
  source 1..44: mol_type = other RNA
aggggtaatacagtacccaggaaccagcgctccaccccatccaa

SEQ ID NO: 68 - RNA, 52 nt, synthetic construct
  source 1..52: mol_type = other RNA
aggaaccagcgctccaccccatccaaacagtgcaaggattaaaggtagcaaa

SEQ ID NO: 69 - RNA, 52 nt, synthetic construct
  source 1..52: mol_type = other RNA
acagtgcaaggattaaaggtagcaaaaggaaccagcgctccaccccatccaa

SEQ ID NO: 70 - RNA, 52 nt, synthetic construct
  source 1..52: mol_type = other RNA
aggaaccagcgctccaccccatccaaaggattaaaggtagcaaaaggggtaa

SEQ ID NO: 71 - RNA, 52 nt, synthetic construct
  source 1..52: mol_type = other RNA
aggattaaaggtagcaaaaggggtaaaggaaccagcgctccaccccatccaa

SEQ ID NO: 72 - RNA, 44 nt, synthetic construct
  source 1..44: mol_type = other RNA
aggaaccagcgctccaccccatccaaagtacccataataaaggg

SEQ ID NO: 73 - RNA, 44 nt, synthetic construct
  source 1..44: mol_type = other RNA
agtacccataataaagggaggaaccagcgctccaccccatccaa

SEQ ID NO: 74 - RNA, 39 nt, synthetic construct
  source 1..39: mol_type = other RNA
agttggagcaagattatcctaaggggtaatacagtaccc

SEQ ID NO: 75 - RNA, 39 nt, synthetic construct
  source 1..39: mol_type = other RNA
aggggtaatacagtacccagttggagcaagattatccta

SEQ ID NO: 76 - RNA, 47 nt, synthetic construct
  source 1..47: mol_type = other RNA
agttggagcaagattatcctaacagtgcaaggattaaaggtagcaaa

SEQ ID NO: 77 - RNA, 47 nt, synthetic construct
  source 1..47: mol_type = other RNA
acagtgcaaggattaaaggtagcaaaagttggagcaagattatccta

SEQ ID NO: 78 - RNA, 47 nt, synthetic construct
  source 1..47: mol_type = other RNA
agttggagcaagattatcctaaggattaaaggtagcaaaaggggtaa

SEQ ID NO: 79 - RNA, 47 nt, synthetic construct
  source 1..47: mol_type = other RNA
aggattaaaggtagcaaaaggggtaaagttggagcaagattatccta

SEQ ID NO: 80 - RNA, 39 nt, synthetic construct
  source 1..39: mol_type = other RNA
agttggagcaagattatcctaagtacccataataaaggg

SEQ ID NO: 81 - RNA, 39 nt, synthetic construct
  source 1..39: mol_type = other RNA
agtacccataataaagggagttggagcaagattatccta

SEQ ID NO: 82 - RNA, 47 nt, synthetic construct
  source 1..47: mol_type = other RNA
agtcacagtgcaaggattaaaggtagagttggagcaagattatccta

SEQ ID NO: 83 - RNA, 47 nt, synthetic construct
  source 1..47: mol_type = other RNA
agttggagcaagattatcctaagtcacagtgcaaggattaaaggtag

SEQ ID NO: 84 - RNA, 27 nt, synthetic construct
  source 1..27: mol_type = other RNA
ggctttctggctccttaccggaaagcc

SEQ ID NO: 85 - RNA, 27 nt, synthetic construct
  source 1..27: mol_type = other RNA
ggctttctgggaggttaccggaaagcc

SEQ ID NO: 86 - RNA, 28 nt, synthetic construct
  source 1..28: mol_type = other RNA
ggctttctggcctccttaccggaaagcc

SEQ ID NO: 87 - RNA, 28 nt, synthetic construct
  source 1..28: mol_type = other RNA
ggctttctggggaggttaccggaaagcc

SEQ ID NO: 88 - RNA, 27 nt, synthetic construct
  source 1..27: mol_type = other RNA
ggctttctggctggctaccggaaagcc

SEQ ID NO: 89 - RNA, 25 nt, synthetic construct
  source 1..25: mol_type = other RNA
ggctttctggcttccccggaaagcc

SEQ ID NO: 90 - RNA, 27 nt, synthetic construct
  source 1..27: mol_type = other RNA
ggctttctggcttcttcccggaaagcc

SEQ ID NO: 91 - RNA, 27 nt, synthetic construct
  source 1..27: mol_type = other RNA
ggctttctggcaacttaccggaaagcc

SEQ ID NO: 92 - RNA, 27 nt, synthetic construct
  source 1..27: mol_type = other RNA
ggctttctggttcggtaccggaaagcc

SEQ ID NO: 93 - RNA, 28 nt, synthetic construct
  source 1..28: mol_type = other RNA
ggctttctggaagccttaccggaaagcc

SEQ ID NO: 94 - RNA, 27 nt, synthetic construct
  source 1..27: mol_type = other RNA
ggctttctggcttcttaccggaaagcc

SEQ ID NO: 95 - RNA, 29 nt, synthetic construct
  source 1..29: mol_type = other RNA
ggctttctggcctccgccggaaagcccct

SEQ ID NO: 96 - RNA, 30 nt, synthetic construct
  source 1..30: mol_type = other RNA
ggctttctggctccttaccggaaagcccct

SEQ ID NO: 97 - RNA, 29 nt, synthetic construct
  source 1..29: mol_type = other RNA
ggttttctgacctccgtcggaaaacccct

SEQ ID NO: 98 - RNA, 33 nt, synthetic construct
  source 1..33: mol_type = other RNA
ggttttctgacctccttcggtcggaaaacccct

SEQ ID NO: 99 - RNA, 26 nt, synthetic construct
  source 1..26: mol_type = other RNA
ggttttctgacctccgtcggaaaacc

SEQ ID NO: 100 - RNA, 30 nt, synthetic construct
  source 1..30: mol_type = other RNA
ggttttctgacactccgtcggaaaacccct

SEQ ID NO: 101 - RNA, 29 nt, synthetic construct
  source 1..29: mol_type = other RNA
ggttttctgatctccatcggaaaacccct

SEQ ID NO: 102 - RNA, 29 nt, synthetic construct
  source 1..29: mol_type = other RNA
ggttttccgacctccgtcggaaaacccct

SEQ ID NO: 103 - RNA, 30 nt, synthetic construct
  source 1..30: mol_type = other RNA
ggctttctggcactccaccggaaagcccct

SEQ ID NO: 104 - RNA, 30 nt, synthetic construct
  source 1..30: mol_type = other RNA
ggctttctggcactccgccggaaagcccct

SEQ ID NO: 105 - RNA, 29 nt, synthetic construct
  source 1..29: mol_type = other RNA
ggctttctggcctccaccggaaagcccct

SEQ ID NO: 106 - RNA, 29 nt, synthetic construct
  source 1..29: mol_type = other RNA
ggttttccggtctccaccggaaagccccc

SEQ ID NO: 107 - RNA, 29 nt, synthetic construct
  source 1..29: mol_type = other RNA
ggctttccggtctccaccggaaagcccct

SEQ ID NO: 108 - RNA, 29 nt, synthetic construct
  source 1..29: mol_type = other RNA
ggctttccggcctccgccggaaagcccct

SEQ ID NO: 109 - RNA, 29 nt, synthetic construct
  source 1..29: mol_type = other RNA
ggttttccggtctccaccggaaaaccctt

SEQ ID NO: 110 - RNA, 29 nt, synthetic construct
  source 1..29: mol_type = other RNA
ggttttccgtgctcccacggaaaaccctt

SEQ ID NO: 111 - RNA, 29 nt, synthetic construct
  source 1..29: mol_type = other RNA
ggttttccggcctccgccggaaaaccctt

SEQ ID NO: 112 - RNA, 30 nt, synthetic construct
  source 1..30: mol_type = other RNA
ggttttccgtgactcccacggaaaaccctt

SEQ ID NO: 113 - RNA, 30 nt, synthetic construct
  source 1..30: mol_type = other RNA
ggttttccggcactccgccggaaaaccctt

SEQ ID NO: 114 - RNA, 29 nt, synthetic construct
  source 1..29: mol_type = other RNA
ggtcttccggtctcctccggaaggccccc

SEQ ID NO: 115 - RNA, 27 nt, synthetic construct
  source 1..27: mol_type = other RNA
ggtcttccggctccccggaaggccccc

SEQ ID NO: 116 - RNA, 29 nt, synthetic construct
  source 1..29: mol_type = other RNA
ggctttccgtgctcccacggaaagcccct

SEQ ID NO: 117 - RNA, 30 nt, synthetic construct
  source 1..30: mol_type = other RNA
ggctttccgtgactcccacggaaagcccct

SEQ ID NO: 118 - RNA, 30 nt, synthetic construct
  source 1..30: mol_type = other RNA
ggctttccggcactccgccggaaagcccct

SEQ ID NO: 119 - RNA, 29 nt, synthetic construct
  source 1..29: mol_type = other RNA
ggttttctgacttcggtcggaaaacccct

SEQ ID NO: 120 - RNA, 26 nt, synthetic construct
  source 1..26: mol_type = other RNA
ggttttctgacttcggtcggaaaacc

SEQ ID NO: 121 - RNA, 27 nt, synthetic construct
  source 1..27: mol_type = other RNA
ggctttctggctttttaccggaaagcc

SEQ ID NO: 122 - RNA, 30 nt, synthetic construct
  source 1..30: mol_type = other RNA
ggctttctggctttttaccggaaagcccct

SEQ ID NO: 123 - RNA, 29 nt, synthetic construct
  source 1..29: mol_type = other RNA
ggctttccggcctccgccggaaagcccct

SEQ ID NO: 124 - RNA, 26 nt, synthetic construct
  source 1..26: mol_type = other RNA
ggctttccggcctccgccggaaagcc

SEQ ID NO: 125 - DNA, 392 bp, Homo sapiens
  source 1..392: mol_type = genomic DNA
aaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 126 - DNA, 247 bp, Homo sapiens
  source 1..247: mol_type = genomic DNA
ccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 127 - DNA, 250 bp, Homo sapiens
  source 1..250: mol_type = genomic DNA
ttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 128 - DNA, 300 bp, Homo sapiens
  source 1..300: mol_type = genomic DNA
gcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 129 - DNA, 296 bp, Homo sapiens
  source 1..296: mol_type = genomic DNA
gtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 130 - DNA, 243 bp, Homo sapiens
  source 1..243: mol_type = genomic DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 131 - DNA, 106 bp, Homo sapiens
  source 1..106: mol_type = genomic DNA
caggactagtcttttaggtcaaaaagaagaagctttgtaaccgttggtttccgtagtgtagtggttatcacgttcgcctaacacgcgaaaggtccccggttcgaag

SEQ ID NO: 132 - DNA, 439 bp, Mus musculus
  source 1..439: mol_type = genomic DNA
ttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 133 - DNA, 258 bp, Mus musculus
  source 1..258: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 134 - DNA, 256 bp, Mus musculus
  source 1..256: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 135 - DNA, 288 bp, Homo sapiens
  source 1..288: mol_type = genomic DNA
tactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 136 - DNA, 440 bp, Mus musculus
  source 1..440: mol_type = genomic DNA
tgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 137 - DNA, 231 bp, Mus musculus
  source 1..231: mol_type = genomic DNA
gggcggggcatgcaaataactgctctgtggaactctgggagcaaaaacaaaaaactgcaaccaaaacttctcggcctccttgaatcttacaggcttttcgtggcgtaaaggtggtgtactcaatgaagaggagagtctgtgttggctgcatgtttgagtcggttggttggtgactgtgaattaaaggtgtggtcggtgttgagtgtatggggcgtgtgggcgtagttcggt

SEQ ID NO: 138 - DNA, 311 bp, Mus musculus
  source 1..311: mol_type = genomic DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttt

SEQ ID NO: 139 - DNA, 97 bp, Mus musculus
  source 1..97: mol_type = genomic DNA
catgcaaattacgcgctgtgctttgtgggaaatcaccctaaacgtaaaatttattcctctttcgagccttatagtggcggccggtctacaccctaaa

SEQ ID NO: 140 - DNA, 50 bp, Homo sapiens
  source 1..50: mol_type = genomic DNA
actttctggagtttcaaaaacagactgtacgccaagggtcatatcttttt

SEQ ID NO: 141 - DNA, 50 bp, Homo sapiens
  source 1..50: mol_type = genomic DNA
ccggggatacaacgtgtttcctaaaagtagagggaggtaagagacggtag

SEQ ID NO: 142 - DNA, 50 bp, Homo sapiens
  source 1..50: mol_type = genomic DNA
ctgaattttcttgcagttgaacaacagaggctttttttgtgtgtgtgggg

SEQ ID NO: 143 - DNA, 50 bp, Homo sapiens
  source 1..50: mol_type = genomic DNA
atatgtggtaatccaacaatagaaattatttttaagtttgtgtgttcctt

SEQ ID NO: 144 - DNA, 206 bp, Mus musculus
  source 1..206: mol_type = genomic DNA
gtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagca

SEQ ID NO: 145 - DNA, 110 bp, Mus musculus
  source 1..110: mol_type = genomic DNA
cccaatttcactggtctacaatgaaagcaaaacagttctcttccccgctccccggtgtgtgagaggggctttgatccttctctggtttcctaggaaacgcgtatgtgtac

SEQ ID NO: 146 - DNA, 107 bp, Mus musculus
  source 1..107: mol_type = genomic DNA
cccaatttcactggtctacaatgaaagcaaaacagttctcttccccgctccccggtgtgtgagaggggctttgatccttctctggtttcctaggaaacgcgtatgtg

SEQ ID NO: 147 - DNA, 94 bp, Homo sapiens
  source 1..94: mol_type = genomic DNA
cctcttatgatgtttgttgccaatgatagattgttttcactgtgcaaaaattatgggtagttttggtggtcttgatgcagttgtaagcttggag

SEQ ID NO: 148 - DNA, 91 bp, Homo sapiens
  source 1..91: mol_type = genomic DNA
cttatgatgtttgttgccaatgatagattgttttcactgtgcaaaaattatgggtagttttggtggtcttgatgcagttgtaagcttggag

SEQ ID NO: 149 - DNA, 200 bp, Mus musculus
  source 1..200: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttga

SEQ ID NO: 150 - DNA, 78 bp, Mus musculus
  source 1..78: mol_type = genomic DNA
ccctctggggagtaaagttggttttaaagtcagagcatggtgattgtagggcagtccaacttttttaaatatgctgtg

SEQ ID NO: 151 - DNA, 30 bp, synthetic construct
  source 1..30: mol_type = other DNA
caaactacagagccaagtgctatccacaga

SEQ ID NO: 152 - DNA, 30 bp, synthetic construct
  source 1..30: mol_type = other DNA
gagctttctgggttgccatctcaagcagac

SEQ ID NO: 153 - DNA, 30 bp, synthetic construct
  source 1..30: mol_type = other DNA
tacaaggccatcagctcatactcacaattg

SEQ ID NO: 154 - DNA, 100 bp, synthetic construct
  source 1..100: mol_type = other DNA
caaactacagagccaagtgctatccacagagagctttctgggttgccatctcaagcagactacaaggccatcagctcatactcacaattgactttgagag

SEQ ID NO: 155 - DNA, 100 bp, synthetic construct
  source 1..100: mol_type = other DNA
ttgaccacatacgtgctctttcaaagttctgtgtttgaagttatgttagtaacaactgatgcccatcctgcaatgacaaatccaattctcagtgcagctc

SEQ ID NO: 156 - DNA, 500 bp, synthetic construct
  source 1..500: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgt

SEQ ID NO: 157 - DNA, 500 bp, synthetic construct
  source 1..500: mol_type = other DNA
ttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaac

SEQ ID NO: 158 - DNA, 110 bp, synthetic construct
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 159 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 160 - RNA, 13 nt, synthetic construct
  source 1..13: mol_type = other RNA
aatttttggagca

SEQ ID NO: 161 - RNA, 13 nt, synthetic construct
  source 1..13: mol_type = other RNA
aatttttggagta

SEQ ID NO: 162 - DNA, 1504 bp, synthetic construct
  source 1..1504: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtaggggtaatacagtacccccaagttggagcaagattatcctatacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtaggggtaatacagtacccccaagttggagcaagattatcctatacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 163 - DNA, 1490 bp, synthetic construct
  source 1..1490: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgttggagcaagattatcctaaggggtaatacagtacccaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgttggagcaagattatcctaaggggtaatacagtacccaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 164 - DNA, 1508 bp, synthetic construct
  source 1..1508: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtaaaggggtaatacagtacccccaagttggagcaagattatcctatacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtaaaggggtaatacagtacccccaagttggagcaagattatcctatacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 165 - DNA, 1492 bp, synthetic construct
  source 1..1492: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtagttggagcaagattatcctaaggggtaatacagtacccaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtagttggagcaagattatcctaaggggtaatacagtacccaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 166 - DNA, 1492 bp, synthetic construct
  source 1..1492: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtaggggtaatacagtacccagttggagcaagattatcctaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtaggggtaatacagtacccagttggagcaagattatcctaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 167 - DNA, 1492 bp, synthetic construct
  source 1..1492: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtagttggagcaagattatcctaagtacccataataaagggaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtagttggagcaagattatcctaagtacccataataaagggaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 168 - DNA, 1492 bp, synthetic construct
  source 1..1492: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtagtacccataataaagggagttggagcaagattatcctaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtagtacccataataaagggagttggagcaagattatcctaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 169 - DNA, 1508 bp, synthetic construct
  source 1..1508: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtagtcacagtgcaaggattaaaggtagagttggagcaagattatcctaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtagtcacagtgcaaggattaaaggtagagttggagcaagattatcctaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 170 - DNA, 1508 bp, synthetic construct
  source 1..1508: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtagttggagcaagattatcctaagtcacagtgcaaggattaaaggtagaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtagttggagcaagattatcctaagtcacagtgcaaggattaaaggtagaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 171 - DNA, 1494 bp, synthetic construct
  source 1..1494: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaaggggtaatacagtacccccaagttggagcaagattatcctatacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaggggtaatacagtacccccaagttggagcaagattatcctatacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 172 - DNA, 1480 bp, synthetic construct
  source 1..1480: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcctaaggggtaatacagtacccaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcgttggagcaagattatcctaaggggtaatacagtacccaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 173 - DNA, 1500 bp, synthetic construct
  source 1..1500: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaaggggtaatacagtacccccaagttggagcaagattatcctatacaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaggggtaatacagtacccccaagttggagcaagattatcctatacaatttttggagcaggttttctgacttcggtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 174 - DNA, 1486 bp, synthetic construct
  source 1..1486: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcctaaggggtaatacagtacccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcgttggagcaagattatcctaaggggtaatacagtacccaatttttggagcaggttttctgacttcggtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 175 - DNA, 1494 bp, synthetic construct
  source 1..1494: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaaggggtaatacagtacccccaagttggagcaagattatcctatacaatttttggagcaggttttctgacttcggtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaggggtaatacagtacccccaagttggagcaagattatcctatacaatttttggagcaggttttctgacttcggtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 176 - DNA, 1480 bp, synthetic construct
  source 1..1480: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcctaaggggtaatacagtacccaatttttggagcaggttttctgacttcggtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcgttggagcaagattatcctaaggggtaatacagtacccaatttttggagcaggttttctgacttcggtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 177 - DNA, 1500 bp, synthetic construct
  source 1..1500: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaaggggtaatacagtacccccaagttggagcaagattatcctatacaatttttggagcaggctttccggcctccgccggaaagcccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaggggtaatacagtacccccaagttggagcaagattatcctatacaatttttggagcaggctttccggcctccgccggaaagcccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 178 - DNA, 1486 bp, synthetic construct
  source 1..1486: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcctaaggggtaatacagtacccaatttttggagcaggctttccggcctccgccggaaagcccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcgttggagcaagattatcctaaggggtaatacagtacccaatttttggagcaggctttccggcctccgccggaaagcccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 179 - DNA, 1494 bp, synthetic construct
  source 1..1494: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaaggggtaatacagtacccccaagttggagcaagattatcctatacaatttttggagcaggctttccggcctccgccggaaagcccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaggggtaatacagtacccccaagttggagcaagattatcctatacaatttttggagcaggctttccggcctccgccggaaagccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 180 - DNA, 1480 bp, synthetic construct
  source 1..1480: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcctaaggggtaatacagtacccaatttttggagcaggctttccggcctccgccggaaagcccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcgttggagcaagattatcctaaggggtaatacagtacccaatttttggagcaggctttccggcctccgccggaaagccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 181 - RNA, 30 nt, synthetic construct
  source 1..30: mol_type = other RNA
aaaaacagacaccagggagagacagagatg

SEQ ID NO: 182 - RNA, 18 nt, synthetic construct
  source 1..18: mol_type = other RNA
gacaccagggagagacag

SEQ ID NO: 183 - RNA, 30 nt, synthetic construct
  source 1..30: mol_type = other RNA
agaaaaacagacaccagggagagacagaga

SEQ ID NO: 184 - RNA, 30 nt, synthetic construct
  source 1..30: mol_type = other RNA
agaagaaaaacagacaccagggagagacag

SEQ ID NO: 185 - RNA, 25 nt, synthetic construct
  source 1..25: mol_type = other RNA
aaacagacaccagggagagacagag

SEQ ID NO: 186 - RNA, 30 nt, synthetic construct
  source 1..30: mol_type = other RNA
gggagagacacgtgggagagagatggaggg

SEQ ID NO: 187 - RNA, 30 nt, synthetic construct
  source 1..30: mol_type = other RNA
aggggagagacacgtgggagagagatggag

SEQ ID NO: 188 - RNA, 30 nt, synthetic construct
  source 1..30: mol_type = other RNA
gaggggagagacacgtgggagagagatgga

SEQ ID NO: 189 - RNA, 30 nt, synthetic construct
  source 1..30: mol_type = other RNA
tgaggggagagacacgtgggagagagatgg

SEQ ID NO: 190 - RNA, 30 nt, synthetic construct
  source 1..30: mol_type = other RNA
gtgaggggagagacacgtgggagagagatg

SEQ ID NO: 191 - RNA, 30 nt, synthetic construct
  source 1..30: mol_type = other RNA
ggtgaggggagagacacgtgggagagagat

SEQ ID NO: 192 - RNA, 30 nt, synthetic construct
  source 1..30: mol_type = other RNA
gagagacacgtgggagagagatggaggggt

SEQ ID NO: 193 - RNA, 30 nt, synthetic construct
  source 1..30: mol_type = other RNA
aaggtgaggggagagacacgtgggagagag

SEQ ID NO: 194 - RNA, 24 nt, synthetic construct
  source 1..24: mol_type = other RNA
gagagacacgtgggagagagatgg

SEQ ID NO: 195 - RNA, 28 nt, synthetic construct
  source 1..28: mol_type = other RNA
aggggagagacacgtgggagagagatgg

SEQ ID NO: 196 - RNA, 25 nt, synthetic construct
  source 1..25: mol_type = other RNA
ggagagacacgtgggagagagatgg

SEQ ID NO: 197 - RNA, 20 nt, synthetic construct
  source 1..20: mol_type = other RNA
gacacgtgggagagagatgg

SEQ ID NO: 198 - RNA, 30 nt, synthetic construct
  source 1..30: mol_type = other RNA
gaccctcagcttccagggaacatgctgagg

SEQ ID NO: 199 - RNA, 30 nt, synthetic construct
  source 1..30: mol_type = other RNA
ctcagcttccagggaacatgctgagggggg

SEQ ID NO: 200 - RNA, 30 nt, synthetic construct
  source 1..30: mol_type = other RNA
ccctcagcttccagggaacatgctgagggg

SEQ ID NO: 201 - RNA, 30 nt, synthetic construct
  source 1..30: mol_type = other RNA
gagaccctcagcttccagggaacatgctga

SEQ ID NO: 202 - RNA, 24 nt, synthetic construct
  source 1..24: mol_type = other RNA
gcttccagggaacatgctgagggg

SEQ ID NO: 203 - RNA, 18 nt, synthetic construct
  source 1..18: mol_type = other RNA
tccagggaacatgctgag

SEQ ID NO: 204 - RNA, 30 nt, synthetic construct
  source 1..30: mol_type = other RNA
cagcttccagggaacatgctgaggggggtg

SEQ ID NO: 205 - RNA, 30 nt, synthetic construct
  source 1..30: mol_type = other RNA
gagagaaagagagagaccgggactgagccc

SEQ ID NO: 206 - RNA, 60 nt, synthetic construct
  source 1..60: mol_type = other RNA
aaaaacagacaccagggagagacagagatggagagacacgtgggagagagatggaggggt

SEQ ID NO: 207 - RNA, 60 nt, synthetic construct
  source 1..60: mol_type = other RNA
aaaaacagacaccagggagagacagagatggaggggagagacacgtgggagagagatgga

SEQ ID NO: 208 - RNA, 60 nt, synthetic construct
  source 1..60: mol_type = other RNA
gagagacacgtgggagagagatggaggggtcagcttccagggaacatgctgaggggggtg

SEQ ID NO: 209 - RNA, 60 nt, synthetic construct
  source 1..60: mol_type = other RNA
gagagacacgtgggagagagatggaggggtccctcagcttccagggaacatgctgagggg

SEQ ID NO: 210 - RNA, 60 nt, synthetic construct
  source 1..60: mol_type = other RNA
gaggggagagacacgtgggagagagatggacagcttccagggaacatgctgaggggggtg

SEQ ID NO: 211 - RNA, 60 nt, synthetic construct
  source 1..60: mol_type = other RNA
gaggggagagacacgtgggagagagatggaccctcagcttccagggaacatgctgagggg

SEQ ID NO: 212 - RNA, 60 nt, synthetic construct
  source 1..60: mol_type = other RNA
cagcttccagggaacatgctgaggggggtggagagacacgtgggagagagatggaggggt

SEQ ID NO: 213 - RNA, 60 nt, synthetic construct
  source 1..60: mol_type = other RNA
ccctcagcttccagggaacatgctgagggggagagacacgtgggagagagatggaggggt

SEQ ID NO: 214 - RNA, 60 nt, synthetic construct
  source 1..60: mol_type = other RNA
cagcttccagggaacatgctgaggggggtggaggggagagacacgtgggagagagatgga

SEQ ID NO: 215 - RNA, 60 nt, synthetic construct
  source 1..60: mol_type = other RNA
ccctcagcttccagggaacatgctgagggggaggggagagacacgtgggagagagatgga

SEQ ID NO: 216 - RNA, 60 nt, synthetic construct
  source 1..60: mol_type = other RNA
aaaaacagacaccagggagagacagagatgccctcagcttccagggaacatgctgagggg

SEQ ID NO: 217 - RNA, 18 nt, synthetic construct
  source 1..18: mol_type = other RNA
cacgtgggagagagatgg

SEQ ID NO: 218 - RNA, 36 nt, synthetic construct
  source 1..36: mol_type = other RNA
ccctcagcttccagggaacatgctgaggggggtggt

SEQ ID NO: 219 - RNA, 40 nt, synthetic construct
  source 1..40: mol_type = other RNA
gaccctcagcttccagggaacatgctgaggggggtggtag

SEQ ID NO: 220 - RNA, 40 nt, synthetic construct
  source 1..40: mol_type = other RNA
gcttccagggaacatgctgaggggggtggtaggaggtgag

SEQ ID NO: 221 - RNA, 60 nt, synthetic construct
  source 1..60: mol_type = other RNA
aagagagagaccgggactgagccccagagaccctcagcttccagggaacatgctgagggg

SEQ ID NO: 222 - RNA, 30 nt, synthetic construct
  source 1..30: mol_type = other RNA
gcttccagggaacatgctgaggggggtggt

SEQ ID NO: 223 - RNA, 30 nt, synthetic construct
  source 1..30: mol_type = other RNA
tccagggaacatgctgaggggggtggtagg

SEQ ID NO: 224 - RNA, 18 nt, synthetic construct
  source 1..18: mol_type = other RNA
gagagagaccgggactga

SEQ ID NO: 225 - RNA, 30 nt, synthetic construct
  source 1..30: mol_type = other RNA
gagaaagagagagaccgggactgagcccca

SEQ ID NO: 226 - RNA, 30 nt, synthetic construct
  source 1..30: mol_type = other RNA
ggaccgcaggagcaagatgggaggctgctt

SEQ ID NO: 227 - RNA, 30 nt, synthetic construct
  source 1..30: mol_type = other RNA
gggaccgcaggagcaagatgggaggctgct

SEQ ID NO: 228 - RNA, 30 nt, synthetic construct
  source 1..30: mol_type = other RNA
agggaccgcaggagcaagatgggaggctgc

SEQ ID NO: 229 - RNA, 30 nt, synthetic construct
  source 1..30: mol_type = other RNA
gagggaccgcaggagcaagatgggaggctg

SEQ ID NO: 230 - RNA, 30 nt, synthetic construct
  source 1..30: mol_type = other RNA
ggagggaccgcaggagcaagatgggaggct

SEQ ID NO: 231 - RNA, 35 nt, synthetic construct
  source 1..35: mol_type = other RNA
ggaccgcaggagcaagatgggaggctgcttgaaga

SEQ ID NO: 232 - RNA, 25 nt, synthetic construct
  source 1..25: mol_type = other RNA
gcaggagcaagatgggaggctgctt

SEQ ID NO: 233 - RNA, 18 nt, synthetic construct
  source 1..18: mol_type = other RNA
gagacacgtgggagagag

SEQ ID NO: 234 - RNA, 24 nt, synthetic construct
  source 1..24: mol_type = other RNA
agacacgtgggagagagatggagg

SEQ ID NO: 235 - RNA, 40 nt, synthetic construct
  source 1..40: mol_type = other RNA
aggtgaggggagagacacgtgggagagagatggaggggtg

SEQ ID NO: 236 - RNA, 24 nt, synthetic construct
  source 1..24: mol_type = other RNA
aggggagagacacgtgggagagag

SEQ ID NO: 237 - RNA, 24 nt, synthetic construct
  source 1..24: mol_type = other RNA
cacgtgggagagagatggaggggt

SEQ ID NO: 238 - RNA, 54 nt, synthetic construct
  source 1..54: mol_type = other RNA
gcttccagggaacatgctgagggggagagaaagagagagaccgggactgagccc

SEQ ID NO: 239 - RNA, 48 nt, synthetic construct
  source 1..48: mol_type = other RNA
tccagggaacatgctgaggagagaaagagagagaccgggactgagccc

SEQ ID NO: 240 - RNA, 60 nt, synthetic construct
  source 1..60: mol_type = other RNA
cagcttccagggaacatgctgaggggggtggagagaaagagagagaccgggactgagccc

SEQ ID NO: 241 - RNA, 54 nt, synthetic construct
  source 1..54: mol_type = other RNA
gagagaaagagagagaccgggactgagcccgcttccagggaacatgctgagggg

SEQ ID NO: 242 - RNA, 48 nt, synthetic construct
  source 1..48: mol_type = other RNA
gagagaaagagagagaccgggactgagccctccagggaacatgctgag

SEQ ID NO: 243 - RNA, 60 nt, synthetic construct
  source 1..60: mol_type = other RNA
gagagaaagagagagaccgggactgagccccagcttccagggaacatgctgaggggggtg

SEQ ID NO: 244 - RNA, 54 nt, synthetic construct
  source 1..54: mol_type = other RNA
gagagacacgtgggagagagatggcagcttccagggaacatgctgaggggggtg

SEQ ID NO: 245 - RNA, 54 nt, synthetic construct
  source 1..54: mol_type = other RNA
cagcttccagggaacatgctgaggggggtggagagacacgtgggagagagatgg

SEQ ID NO: 246 - RNA, 55 nt, synthetic construct
  source 1..55: mol_type = other RNA
ggagagacacgtgggagagagatggcagcttccagggaacatgctgaggggggtg

SEQ ID NO: 247 - RNA, 43 nt, synthetic construct
  source 1..43: mol_type = other RNA
ggagagacacgtgggagagagatggtccagggaacatgctgag

SEQ ID NO: 248 - RNA, 55 nt, synthetic construct
  source 1..55: mol_type = other RNA
cagcttccagggaacatgctgaggggggtgggagagacacgtgggagagagatgg

SEQ ID NO: 249 - RNA, 43 nt, synthetic construct
  source 1..43: mol_type = other RNA
tccagggaacatgctgagggagagacacgtgggagagagatgg

SEQ ID NO: 250 - RNA, 36 nt, synthetic construct
  source 1..36: mol_type = other RNA
tccagggaacatgctgagtccagggaacatgctgag

SEQ ID NO: 251 - RNA, 20 nt, synthetic construct
  source 1..20: mol_type = other RNA
agacaccagggagagacaga

SEQ ID NO: 252 - RNA, 19 nt, synthetic construct
  source 1..19: mol_type = other RNA
cagacaccagggagagaca

SEQ ID NO: 253 - RNA, 19 nt, synthetic construct
  source 1..19: mol_type = other RNA
acacgtgggagagagatgg

SEQ ID NO: 254 - RNA, 20 nt, synthetic construct
  source 1..20: mol_type = other RNA
acacgtgggagagagatgga

SEQ ID NO: 255 - RNA, 22 nt, synthetic construct
  source 1..22: mol_type = other RNA
gagacacgtgggagagagatgg

SEQ ID NO: 256 - RNA, 19 nt, synthetic construct
  source 1..19: mol_type = other RNA
gagagagaccgggactgag

SEQ ID NO: 257 - RNA, 18 nt, synthetic construct
  source 1..18: mol_type = other RNA
ggggggaagggtcgggga

SEQ ID NO: 258 - RNA, 20 nt, synthetic construct
  source 1..20: mol_type = other RNA
ctggggggaagggtcgggga

SEQ ID NO: 259 - RNA, 30 nt, synthetic construct
  source 1..30: mol_type = other RNA
cgggaacacgctggggggaagggtcgggga

SEQ ID NO: 260 - RNA, 30 nt, synthetic construct
  source 1..30: mol_type = other RNA
acctccttcagctccctcgggaacacgctg

SEQ ID NO: 261 - RNA, 37 nt, synthetic construct
  source 1..37: mol_type = other RNA
tccagggaacatgctgaggagagagaccgggactgag

SEQ ID NO: 262 - RNA, 37 nt, synthetic construct
  source 1..37: mol_type = other RNA
gagagagaccgggactgagtccagggaacatgctgag

SEQ ID NO: 263 - RNA, 49 nt, synthetic construct
  source 1..49: mol_type = other RNA
cagcttccagggaacatgctgaggggggtggagagagaccgggactgag

SEQ ID NO: 264 - RNA, 49 nt, synthetic construct
  source 1..49: mol_type = other RNA
gagagagaccgggactgagcagcttccagggaacatgctgaggggggtg

SEQ ID NO: 265 - RNA, 54 nt, synthetic construct; identical to SEQ ID NO: 244
  source 1..54: mol_type = other RNA

SEQ ID NO: 266 - RNA, 50 nt, synthetic construct
  source 1..50: mol_type = other RNA
ctggggggaagggtcggggacagcttccagggaacatgctgaggggggtg

SEQ ID NO: 267 - RNA, 38 nt, synthetic construct
  source 1..38: mol_type = other RNA
ctggggggaagggtcggggatccagggaacatgctgag

SEQ ID NO: 268 - RNA, 38 nt, synthetic construct
  source 1..38: mol_type = other RNA
tccagggaacatgctgagctggggggaagggtcgggga

SEQ ID NO: 269 - RNA, 50 nt, synthetic construct
  source 1..50: mol_type = other RNA
cagcttccagggaacatgctgaggggggtgctggggggaagggtcgggga

SEQ ID NO: 270 - RNA, 60 nt, synthetic construct
  source 1..60: mol_type = other RNA
cgggaacacgctggggggaagggtcggggacagcttccagggaacatgctgaggggggtg

SEQ ID NO: 271 - RNA, 48 nt, synthetic construct
  source 1..48: mol_type = other RNA
cgggaacacgctggggggaagggtcggggatccagggaacatgctgag

SEQ ID NO: 272 - RNA, 39 nt, synthetic construct
  source 1..39: mol_type = other RNA
gagagagaccgggactgagctggggggaagggtcgggga

SEQ ID NO: 273 - RNA, 39 nt, synthetic construct
  source 1..39: mol_type = other RNA
ctggggggaagggtcggggagagagagaccgggactgag

SEQ ID NO: 274 - RNA, 50 nt, synthetic construct
  source 1..50: mol_type = other RNA
ctggggggaagggtcggggacgggaacacgctggggggaagggtcgggga

SEQ ID NO: 275 - RNA, 44 nt, synthetic construct
  source 1..44: mol_type = other RNA
gagagacacgtgggagagagatggctggggggaagggtcgggga

SEQ ID NO: 276 - RNA, 44 nt, synthetic construct
  source 1..44: mol_type = other RNA
ctggggggaagggtcggggagagagacacgtgggagagagatgg

SEQ ID NO: 277 - RNA, 42 nt, synthetic construct
  source 1..42: mol_type = other RNA
tccagggaacatgctgaggagagacacgtgggagagagatgg

SEQ ID NO: 278 - RNA, 42 nt, synthetic construct
  source 1..42: mol_type = other RNA
gagagacacgtgggagagagatggtccagggaacatgctgag

SEQ ID NO: 279 - RNA, 38 nt, synthetic construct
  source 1..38: mol_type = other RNA
ctggggggaagggtcggggacacgtgggagagagatgg

SEQ ID NO: 280 - RNA, 6015 nt, Homo sapiens
  source 1..6015: mol_type = genomic RNA
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctccatcgaccttcagtccttcatggctcgaggcctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 281 - DNA, 420 bp, Rattus norvegicus
  source 1..420: mol_type = genomic DNA
ttacttcatactaaaggctgtgcatccgactcctaagttgatgaaggaaaatgcctagtgttcttggaggctacaaaacaaaagacaaagctaactaccatctgcttatgggttcattggtattttccagctggcagggaggcgggtttccgagtacaggaaatgagtctctatggaggcggtgctatgtagatgagaattcaggagcaaactgggaaaagcagctgcttccaaatatttgtgatttttggagtgtagttttggggaaactcgcagcctaccaattctcctaagtgctttagaatatggagagacactgtacataaagatatagagctttttaatggaggcttaaatttataccgtatctacaaaatgctacattcacaatgcagttcaggctctgtggcattgcaactc

SEQ ID NO: 282 - DNA, 306 bp, Bos taurus
  source 1..306: mol_type = genomic DNA
gcgcaggagccgccgagctcttgctgcgaagccttgtctgcgttcttaaaaactaaagggggcgtgactggcttccttatcagccaatcggcatcgggtcatttgcatagggccccatccaccgttcgtaaactctagccgcgaaggatttaagagagttccttagaacacggtcttcccctccgaaagtcgcagtctgcctcgttatctgtagagcaataggagctaggaaccggccgtcggggctcaccctcactgacagcggggttggtggctatgccgtgcgatctccgggtgctacgtctg

SEQ ID NO: 283 - DNA, 420 bp, synthetic construct
  source 1..420: mol_type = other DNA
cgccgtgccctgctctgccctccgcacgctgctcagactccacacccgtaacgaagctccaccgaatttttcgtctgtctgtgaagacgaagaattgaaaaatctcagctacacttggtctttggggtcggcgccaggcagagctagaacgaactagacgcttgtccgaaagcagtgacgtcaaatgacaagggacaagaggtggggatatgtagatgaggggagcggtggctgctggctggatttcgtctttcgccgggtagaatttcccctgaacgtccaaaggatgtatcttggtcactgtgaaataaataatccttcaggttcccatcgaaggccgaaatagcttggaatataattgaccataactctgttgtgtgctgagttcgagggtgacgtcccagactaagcttacaactc

SEQ ID NO: 284 - DNA, 420 bp, Equus caballus
  source 1..420: mol_type = genomic DNA
tgccgcgcagaatgcagactcatcctgacaagtccacgccagaacctttgcgtctgcccggaaaggcaaggaagtttaaagcctttccagatacccctatggatgattgtcttttagatcgtcctgagggctagctagagcgaattcaccgcttctccgagaagcagaatgacgtcgagtgacgacagtgatgacaagaggcggggatatgtagatgaaagacacggtgactgccggttggagtgagttcttcatggggaacactttccactgaatatccaaaaggatgtaccctgatctctgtcatataaataacccttcaatttcgaacttaaggcctaaagaactcggaatataattgaccatgactttatagtgtgttgaagacgatggtgaggtgccaggcgaggggagcaactc

SEQ ID NO: 285 - RNA, 200 nt, Rattus norvegicus
  source 1..200: mol_type = genomic RNA
atttgtttggtactaaagatagttatcagccgaaccagaaggctaaaatggcttctgatacttacttggccaatgccttttccctttatactgctattgctttgtattctgaaaagcatcttagtggtttttaactttctctacgttccctctgcacgttgagtcttgagttatgttaaatggtacggtacctaggattg

SEQ ID NO: 286 - DNA, 200 bp, Bos taurus
  source 1..200: mol_type = genomic DNA
cccacacaagtgtttatgaacaatagaataatatgtgggtgggaagcttggttttttttgatacgggttttttttttttttttggtctacgtgtttagtccgttcttgacggagaagacccgggaggttgggattcttgtctggaaatttccggctctgtctgatgtagtttcccgttcgcctcccttctttgctgcgag

SEQ ID NO: 287 - DNA, 200 bp, synthetic construct
  source 1..200: mol_type = other DNA
gcgtggtttgtgcaaggaaagacatagatgctaagtttggagctctgctaatttcctgtttttgtagtcgttttcgggtttatgaggcagaggcagtaagcagttttcctgcaacatggataagctagtttggacttttggtaaaggatttttttgtaatgtgggagacttgcttttgatccctggtttgggaaattctc

SEQ ID NO: 288 - DNA, 200 bp, Equus caballus
  source 1..200: mol_type = genomic DNA
acaaacttggtcttaaaagaagactacttgtttatgttcttctttctctaacttttatttggggtttagtggctttctaacatggtcattatgaggtaagaccttggggttggcctattaattaacaccttggggctggccgtatggctgagtggttaagtttgtgtgctctgcttcgggtttggcctgttctaattctg

(19) United States
(12) Patent Application Publication
(10) Pub. No.: US 2026/0234627 A1
(43) Pub. Date: Aug. 13, 2026

(54) COMPOSITIONS AND METHODS COMPRISING SMALL NUCLEAR RNA (SNRNA) FOR TREATING GENETIC EPILEPSIES

(71) Applicant: Regeneron Pharmaceuticals, Inc., Tarrytown, NY (US)

(72) Inventors: Daniela ROTH, San Diego, CA (US); Ranjan BATRA, San Diego, CA (US); Rea LARDELLI MARKMILLER, San Diego, CA (US); Rachel A. ADAMS, San Diego, CA (US); Melissa PARKER, San Diego, CA (US); Daniel A. KNOWLAND, San Diego, CA (US)

(21) Appl. No.: 19/655,027

(22) Filed: Apr. 22, 2026

Related U.S. Application Data
(63) Continuation of application No. PCT/US2024/052599, filed on Oct. 23, 2024.
(60) Provisional application No. 63/592,405, filed on Oct. 23, 2023.
(60) Provisional application No. 63/592,727, filed on Oct. 24, 2023.

(51) Int. Cl. C12N 15/113; C12N 15/86

(52) U.S. Cl. CPC C12N 15/113; C12N 15/86; C12N 2310/531; C12N 2320/33; C12N 2750/14143

(57) ABSTRACT

SnRNA systems targeting SCN1A or SYNGAP1 RNA sequences are disclosed herein. Further disclosed are methods of treating genetic epilepsies such as Dravet Syndrome or SYNGAP1-related epilepsy.

### RELATED APPLICATIONS

This application is a continuation of International Application No. PCT/US2024/052599, filed on Oct. 23, 2024, which claims the priority to, and benefit of, U.S. Provisional Application No. 63/592,405, filed on Oct. 23, 2023 and U.S. Provisional Application No. 63/592,727, filed on Oct. 24, 2023, the contents of each of which are incorporated by reference in their entireties.

### REFERENCE TO AN ELECTRONIC SEQUENCE LISTING

The contents of the electronic sequence listing (REGE_035_C01US_SeqList_ST26.xml; Size: 492,585 bytes; and Date of Creation: Apr. 22, 2026) is herein incorporated by reference in its entirety.

### FIELD OF THE DISCLOSURE

The disclosure is directed to molecular biology, gene therapy, and compositions and methods for modifying expression and activity of RNA molecules.

### BACKGROUND

Small nuclear RNA (snRNA) is one of the smallest types of RNA with an average size of about 150 nucleotides. snRNAs are functional non-coding RNAs. Eucaryotic genomes code for a variety of non-coding RNA such as snRNA, a class of highly abundant RNA, localized in the nucleus with important functions in intron splicing and RNA processing. snRNA, in the pre-mRNA splicing process, are capable of forming ribonucleoprotein particles (snRNPs) along with other proteins. These snRNPs and additional proteins form a large particulate complex (spliceosome) bound to the unspliced pre-mRNA transcripts. In addition to splicing, snRNAs function in nuclear maturation of nascent transcripts, gene expression regulation, as a splice donor in non-canonical systems, and in 3′ end processing of replication-dependent histone mRNAs. U7 snRNA can be programmed to bind and modulate mRNA without exogenous protein expression, this will ultimately decrease the risk of immunogenicity, observed with other protein-based gene therapy approaches. Furthermore, the small size of these programmed snRNAs creates an opportunity to develop single vector, highly specific (allele-specific), single target and multi-targeting gene therapy approaches.

Genetic epilepsies are a class of seizure disorders with an underlying genetic cause via the inherited or spontaneous mutation to a gene.

Dravet Syndrome is a rare developmental and epileptic encephalopathy that usually begins within first year of life. Symptoms of Dravet Syndrome include frequent and prolonged seizures, intellectual disability, developmental delays, behavioral disturbances, and ataxia. Dravet Syndrome patients are also at high risk for sudden unexpected death in epilepsy (SUDEP). The mean age of onset of SUDEP is 4.6 years.

Approximately 80% of patients with Dravet Syndrome have a de novo heterozygous missense mutation or truncation in the SCN1A gene. SCN1A encodes the a subunit of the voltage-gated sodium ion channel type 1 (Nav1.1), which is critical in normal neurological function. Nav1.1 is the primary Na⁺ channel in GABAergic inhibitory interneurons where it induces fast depolarization of neuronal membranes during action potential initiation. Mutations in Nav1.1 result in haploinsufficiency of Nav1.1 protein. The SCN1A gene has 26 exons spanning 6030 bp, making it too large for gene replacement therapy.

Dravet Syndrome is estimated to have a prevalence of about 1:15,700 in US. To date, treatment include anticonvulsant therapies, which only partially manage seizures and do not improve other comorbidities, a ketogenic diet to reduce seizure frequency, and supportive therapies to address neurodevelopmental challenges. Accordingly, there remains an unmet need for new therapeutic approaches for Dravet Syndrome.

SYNGAP1-related epilepsy is a rare genetic disorder caused by a variant on the SYNGAP1 gene. Many individuals develop epilepsy with varying degrees of severity, and/or autism spectrum disorder and other behavioral, social, and sensory issues.

The SYNGAP1 gene is located on chromosome 6 and is responsible for producing the Synaptic Ras GTPase-activating protein 1 (SYNGAP1) protein. High levels of SYNGAP1 are present in the brain and is essential for proper function and development. SYNGAP1 is a key regulator of the neural mechanisms of learning and memory where neurons communicate with each other (synapses). Seizure types associated with SYNGAP1 include: atypical and typical absences, myoclonic seizures, atonic seizures, eyelid myoclonia with absences (EMA), and myoclonic-atonic epilepsy (MAE or Doose syndrome). More frequently, persons have combined features of EMA and MAE. In persons with this gene, a characteristic feature is eyelid myoclonia that evolves to a myoclonic-atonic or atonic seizure.

Seizures often begin around 4 months to 7 years with an average age of 2 years. Seizure triggers are frequent during the active phases of epilepsy, which may reach 100 or more seizures per day in some individuals. Developmental delay or intellectual disability is present in nearly all people affected by SYNGAP1. Typically, developmental delay is evident during the first two years of the child's life. This leads to an intellectual disability diagnosis. Two-thirds of children also exhibit hypotonia (low muscle tone), and a smaller group have ataxia (poor balance) and/or other gait abnormalities. The incidence of SYNGAP1 is estimated to be 1-4 out of 10,000 individuals. Accordingly, there remains an unmet need for new therapeutic approaches for SYNGAP1-related epilepsy.

For some individuals with SYNGAP1-related epilepsy there is a premature termination codon in poison exon 11 of SYNGAP1 leading to nonsense mediated decay, transcript insufficiency and haploinsufficiency thereby leading the patient to experience symptoms associated with SYNGAP1-related epilepsy.

### SUMMARY

cRNA-targeting nucleic acid molecule comprising a small nuclear RNA (snRNA), wherein the snRNA comprises a targeting sequence that binds an SCN1A or SYNGAP1 RNA sequence.

In some embodiments, the SCN1A RNA sequence is Exon 20N. In some embodiments, the SYNGAP1 RNA sequence is Exon 11. In some embodiments, the SCN1A targeting sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 16-83. In some embodiments, the SYNGAP1 targeting sequence comprises the nucleic acid sequence set forth in any one of SEQ ID NOs: 181-279.

In some embodiments, the snRNA comprises an stem loop (SL). In some embodiments, the SL comprises one or more nucleic acid sequences set forth in any one of SEQ ID NOs: 84-124. In some embodiments, the SL comprises one or more nucleic acid sequences that are each, independently, at least 65%, 70%, 75%, 80%, 85%, 90%, 95%, 96%, 97%, 98%, 99% or 100% (or any percentage in between) identical to the nucleic acid sequence set forth in any one of SEQ ID NOs: 84-124. In some embodiments, the SL comprises or consists of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NO: 97.

The disclosure provides an RNA-targeting nucleic acid molecule comprising a small nuclear RNA (snRNA), wherein the snRNA comprises a targeting sequence that binds an SCN1A RNA sequence and a SL comprising the nucleic acid sequence set forth in SEQ ID NO: 97, wherein the targeting sequence is positioned 5′ of the SL.

In some embodiments, the SCN1A RNA sequence is Exon 20N. In some embodiments, the SCN1A targeting sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 16-83

In some embodiments, the SCN1A RNA sequence is a pre-mRNA or mRNA sequence.

In some embodiments, the snRNA comprises an Sm binding domain (SmBD). In some embodiments, the SmBD is a U1, U2, U4, or U5 SmBD. In some embodiments, the SmBD comprises the nucleic acid sequence set forth in any one of SEQ ID NOs: 160 or 161.

In some embodiments, the snRNA comprises a 5′ interaction stabilizer domain (5′ISD). In some embodiments, the 5′ISD comprises the nucleotide sequence ggagt, cctct, ggaggt, cctcct, agccag, ggaag, gaagaag, gttg, ccgaa, taaggag, gaag, or ggctt.

The disclosure provides a vector comprising one or more snRNA of any embodiment of the disclosure. In some embodiments, the vector is an adeno-associated virus (AAV) vector.

In some embodiments, the snRNA is operably linked to a promoter. In some embodiments, the snRNA is operably linked to a U7 promoter or a U1 promoter.

In some embodiments, the snRNA is operably linked to a downstream terminator (DT). In some embodiments, the snRNA is operably linked to a U7 downstream terminator or a U1 downstream terminator.

In some embodiments, the vector comprises at least one, at least two, at least three, at least four, or at least five snRNA. In some embodiments, the least one, at least two, at least three, at least four, or at least five snRNA each target the same target RNA sequences.

In some embodiments, each snRNA is separated by a buffer sequence. In some embodiments, the buffer sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one SEQ ID NOs: 151-157.

In some embodiments, the vector comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 162-180.

The disclosure provides a method of targeting one or more target RNAs of interest and exon-skipping the one or more target RNAs, comprising contacting the snRNA of any embodiment of the disclosure with a cell comprising the one or more target RNAs.

The disclosure provides an SCN1A RNA-targeting nucleic acid molecule comprising a targeting sequence set forth in any one of SEQ ID NOs: 16-83. The disclosure provides a SYNGAP1 RNA-targeting nucleic acid molecule comprising a targeting sequence set forth in any one of SEQ ID NOs: 181-279.

The disclosure provides a polynucleotide or vector comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 162-180.

The disclosure provides a recombinant AAV (rAAV) comprising: (a) an AAV capsid comprising an AAV capsid protein; and (b) a vector genome comprising a snRNA or RNA-targeting nucleic acid molecule as disclosed herein.

In some embodiments, the vector genome further comprises a 5′ inverted terminal repeat (ITR) sequence and a 3′ ITR. In some embodiments, the vector genome comprises in the 5′ to 3′ direction, a 5′ ITR sequence, the snRNA or the RNA-targeting nucleic acid molecule, and a 3′ ITR sequence.

The disclosure provides a recombinant AAV (rAAV) comprising: an AAV capsid comprising an AAV capsid protein; and a vector genome comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 162-180.

In some embodiments, the AAV capsid comprises an AAV capsid protein of an AAV serotype selected from the group consisting of: AAV1, AAV2, AAV3, AAV4, AAV5, AAV6, AAV7, AAV8, AAV9, AAV10, AAVrh10, AAV11, AAV12, and variants thereof.

In some embodiments, the vector genome is single-stranded or self-complementary.

In some embodiments, the rAAV is replication incompetent.

The disclosure provides a pharmaceutical composition comprising a snRNA, RNA-targeting nucleic acid molecule, vector, polynucleotide, or rAAV as disclosed herein.

A method of targeting one or more target RNAs of interest and exon-skipping the one or more target RNAs, comprising contacting a snRNA, RNA-targeting nucleic acid molecule, vector, polynucleotide, or rAAV as disclosed herein with a cell comprising the one or more target RNAs.

In some embodiments, the one or more target RNAs of interest comprise poison exon 20N of a human sodium channel Nav1.1 gene.

The disclosure provides a method of treating a disease or disorder in a subject comprising administering to the subject a snRNA, RNA-targeting nucleic acid molecule, vector, polynucleotide, rAAV, or pharmaceutical composition disclosed herein.

In some embodiments, the disease or disorder is a genetic epilepsy. In some embodiments, the disease or disorder is Dravet Syndrome. In some embodiments, the disease or disorder is SYNGAP1-related epilepsy.

In some embodiments, the administration is systemic, intravenous, or intracerebroventricular.

The disclosure provides a use of a snRNA or RNA-targeting nucleic acid molecule as disclosed herein for targeting one or more target RNAs of interest and exon-skipping the one or more target RNAs.

In some embodiments, the one or more target RNAs are comprised in a cell.

The disclosure provides a use of a snRNA, RNA-targeting nucleic acid molecule, vector, polynucleotide, rAAV, or pharmaceutical composition as disclosure herein for treating a disease or disorder in a subject.

In some embodiments, the disease or disorder is a genetic epilepsy. In some embodiments, the disease or disorder is Dravet Syndrome.

In some embodiments, the administration is systemic, intravenous, or intracerebroventricular.

### BRIEF DESCRIPTION OF THE DRAWINGS

FIG. 1A shows the mechanism of action of SCN1A poison exon 20N skipping by U7 snRNAs to restore frame and function of the sodium channel Nav1.1 in Dravet syndrome. U7 snRNAs bind splicing regulatory sequences within the SCN1A poison exon 20N (acceptor and donor sites or splicing enhancer sequences) to promote skipping and restoration of frame and function. PTC: premature termination codon; NMD: nonsense mediated decay. FIG. 1B shows a tapestation image of the RT-PCR products after U7 snRNA treatment using single U7 spacers (in pcDNA), showing the SCN1A exon 20N non-skipped band (containing the poison exon, top fragment), or the skipped product (exclusion of the poison exon, bottom band). Non-targeting U7 (NT), empty pcDNA plasmid (pc) and untreated cells (UNT) were used as a negative control. FIG. 1C depicts quantification of the gel shown in FIG. 1B plotted as percentage of poison exon 20N inclusion post-treatment with multiple U7 snRNA single-spacers in Hela cells. The dashed line denotes the average of the poison exon 20N inclusion in control conditions as untreated cells or cells transfected with empty plasmid or non-targeting (NT) U7 snRNA.

FIG. 2A shows a tapestation image of the RT-PCR products after U7 snRNA treatments using single and fusion U7 spacers (cloned into pcDNA plasmid), showing the SCN1A exon 20N non-skipped band (containing the poison exon, top fragment), or the skipped product (exclusion of the poison exon, bottom band). Non-targeting U7 (NT), and untreated cells (UNT) were used as a negative control. FIG. 2B depicts a quantification of gel shown in FIG. 2A plotted as percentage of poison exon 20N inclusion post-treatment with multiple U7 snRNA spacers in HepG2 cells. FIG. 2C depicts the expression of productive SCN1A (without the poison exon 20N) determined by qRT-PCR following treatment with single and fusion U7 snRNAs.

FIG. 3A shows a quantification of the RT-PCR products for percentage of poison exon 20N inclusion after U7 snRNA treatments using fusion synthetic U7 spacers of z15/z6 and z11/z15 with additional modifications to the ISD and stem loop. Non-targeting U7 (NT), and untreated cells (UNT) were used as a negative control. FIG. 3B depicts the expression of productive SCN1A (without the poison exon 20N) determined by qRT-PCR following treatment with U7 snRNAs.

FIG. 4A shows a tapestation image of the RT-PCR products for exon 20N skipping detection after U7 snRNA treatments using increasing MOIs of AAV9 (dual U7 snRNA cassettes expressing fusion spacers under mouse U7 and mouse U1 promoters). The gel image shows the SCN1A poison exon 20N (non-skipped band, top), and the skipped products (bottom band). AAV9 null (empty capsid) and untreated (UNT) cells were used as negative controls. FIG. 4B depicts the quantification of the gel shown in FIG. 4A plotted as percentage of poison exon 20N inclusion post-AAV9 transduction with 2 U7 snRNAs vectors A05290 and A05291. FIG. 4C shows quantification of the levels of productive SCN1A RNA (without the poison exon) 7 days post-transduction with AAV9 containing dual U7 snRNA cassettes in SCN1A+/−GABAergic neurons.

FIG. 5A shows a diagram of the study design for in vivo studies of wild type (WT) mice treated with scAAV9-U7 snRNA targeting SCN1A poison exon delivered intracerebroventricularly (ICV) at P0 and harvested at P20. FIG. 5B shows a representative tapestation image for cortex and hippocampus samples of the RT-PCR products after U7 snRNA treatments using 2 lead constructs A05290 and A05291, showing the SCN1A poison exon 21N (the mouse equivalent of poison exon 20N in human) non-skipped band (containing the poison exon, top fragment), or the skipped product (exclusion of the poison exon, bottom band). Vehicle treated samples were used as a negative control. FIG. 5C depicts the quantification of the gel shown in FIG. 5B, with samples from additional cortex slices (1, 2 and 3) and hippocampus slices (1 and 2), plotted as percentage of poison exon 21N inclusion post-AAV9 transduction with 2 U7 snRNAs vectors. FIG. 5D shows the quantification of the levels of productive SCN1A mRNA (i.e., without the poison exon) in. 3 areas of the cortex and 2 areas of the hippocampus 20 days post ICV injection with AAV9 expressing dual U7 snRNA cassettes. FIG. 5E shows SCN1A/Nav1.1 protein expression levels in cortex (CTX) and hippocampus (Hippo) 20 days post ICV injection with AAV9 expressing dual U7 snRNA cassettes. FIG. 5F shows snRNA expression in cortex and hippocampus (Hipp) after AAV9 expressing snRNAs treatment.

FIG. 6A is a schematic depicting the inclusion of SYNGAP poison exon 11 (shown as a hatched box) which contains a premature termination codon (PTC) and leads to nonsense mediated decay (NMD) transcript degradation and haploinsufficiency. FIG. 6B is a schematic showing the mechanism of action of SYNGAP poison exon 11 skipping by U7 snRNAs to restore frame and function. U7 snRNAs were engineered to bind splicing regulatory sequences within the SYNGAP poison exon 11 to promote skipping and restoration of frame and function. FIG. 6C is a tapestation image of the RT-PCR products after U7 snRNA treatments using single U7 spacers (in pcDNA, z22, z23 and z24), showing the SYNGAP poison exon 11 non-skipped band (containing the poison exon, top fragment), or the skipped product (exclusion of the poison exon, bottom band). Non-targeting U7 (NT), was used as a negative control. ASOs previously shown to promote exclusion of poison exon 11 were used as a positive control. FIG. 6D is a graph depicting quantification of tapestation results plotted as percentage of poison exon exclusion post-treatment with multiple single and fusion spacers U7 snRNAs in Hek293-T cells targeting distinct areas of SYNGAP1 pre-mRNA.

FIG. 7A is a graph depicting the quantification of the percentage of endogenous SYNGAP1 poison exon 11 skipping determined by the splicing assay post-treatment with single synthetic U7 snRNAs in HEK293-T cells. FIG. 7B is a graph depicting quantification of the expression of productive SYNGAP1 mRNA determined by qRT-PCR post-treatment with single synthetic U7 snRNAs in HEK293-T cells. Non-targeting U7 (NT) was used as a negative control. FIG. 7C is a graph depicting quantification of the percentage of endogenous SYNGAP1 poison exon 11 skipping determined by the splicing assay post-treatment with new optimized single and fusion U7 snRNAs in HEK293-T cells. FIG. 7D is a graph depicting quantification of the expression of productive SYNGAP1 mRNA determined by qRT-PCR post-treatment with new optimized single and fusion U7 snRNAs in HEK293-T cells. Non-targeting U7 (NT) was used as a negative control.

FIG. 8A is a tapestation image of the RT-PCR products of SYNGAP1 exon 11 levels in patient neural progenitor cells (NPCs) containing the R1240X mutation. Patient NPCS show high levels of SYNGAP1 poison exon 11 inclusion (top band). FIG. 8B is a graph depicting the quantification of the RT-PCR products for percentage of SYNGAP1 poison exon 11 skipping post-U7 snRNA treatments in patient-derived NPCs. Non-targeting U7 (NT) was used as a negative control. FIG. 8C is a graph depicting the quantification of the expression of productive SYNGAP1 mRNA (without the poison exon 11) determined by qRT-PCR following treatment with U7 snRNAs.

### DETAILED DESCRIPTION

The disclosure provides gene therapy compositions comprising a therapeutic RNA-targeting platform comprised of short nuclear RNA (snRNA) targeting precursor mRNA (pre-mRNA) or mRNA sequences encoding Nav1.1 (SCN1A). The targeted pre-mRNA or mRNA sequences can include exonic regions of SCN1A and/or splicing regulatory sequences of SCN1A.

The disclosure provides gene therapy compositions comprising a therapeutic RNA-targeting platform comprised of short nuclear RNA (snRNA) targeting precursor mRNA (pre-mRNA) or mRNA sequences encoding Synaptic Ras GTPase-activating protein 1 (SYNGAP1). The targeted pre-mRNA or mRNA sequences can include exonic regions of SYNGAP1 and/or splicing regulatory sequences of SYNGAP1.

Disclosed herein are compositions comprising nucleic acid molecules, and vectors comprising the snRNA construct or constructs targeting SCN1A. The snRNA molecules of the disclosure can be non-natural, modified and/or engineered snRNA (esnRNA). In some embodiments, snRNA or esnRNA targeting SCN1A of the disclosure comprise a mutated snRNA stem loop. In some embodiments, snRNA targeting SCN1A of the disclosure comprises a native stem loop.

Disclosed herein are compositions comprising nucleic acid molecules, and vectors comprising the snRNA construct or constructs targeting SYNGAP1. The snRNA molecules of the disclosure can be non-natural, modified and/or engineered snRNA (esnRNA). In some embodiments, snRNA or esnRNA targeting SYNGAP1 of the disclosure comprise a mutated snRNA stem loop. In some embodiments, snRNA targeting SYNGAP1 of the disclosure comprises a native stem loop.

Small nuclear ribonucleic acids (snRNAs) are essential components of small nuclear ribonucleoprotein complexes (snRNPs) which, when assembled with additional proteins, form the large ribonucleoprotein complex known as the spliceosome, the cell machinery appointed to mediate the entire mRNA maturation process. The spliceosome is responsible for precursor mRNA splicing; the process that removes introns from RNA transcripts before protein production. An individual snRNA is generally about 250 nucleotides or less in size. For example, U1 snRNA is 164 nucleotides in length and is encoded by genes that occur in several copies within the human genome. U1 snRNA represents the ribonucleic component of the nuclear particle U1 snRNP. The U1 snRNA has a stem and loop tridimensional structure and within the 5′ region there is a single-stranded sequence, generally about 9 nucleotides in length, capable of binding by complementary base pairing to the splicing donor site on the pre-mRNA molecule. (Horowitz et al., 1994, Trends Genet., 10(3):100-6.) The various spliceosomal snRNAs have been designated as U1, U2, U4, U5, U6, U4ATAC, U6ATAC, U7, U11 and U12, due to the generous amount of uridylic acid they contain. (Mattaj et al., 1993, FASEB J, 15, 7:47-53.)

snRNA systems can be used for treating toxic mutations. For example, antisense oligonucleotides that interfere with splice sites and regulatory elements within an exon containing toxic mutations can induce skipping of specific exons at the pre-RNA level. Such antisense sequences can be packaged in an snRNA sequence delivered using viral vectors carrying a nucleic acid sequence from which the snRNA can be transcribed. U7 snRNA is endogenously involved in histone pre-mRNA 3′-end processing but can be converted into a versatile tool for splicing modulation by a small change in the binding site for Sm/Lsm proteins.

Most U-rich snRNPs are complexes that mediate the splicing of pre-mRNAs. U7 snRNP is an exception. U7 is not involved in splicing but rather is a key factor in the unique 3′-end processing of replication-dependent histone mRNAs. By modifying the U7 snRNA histone binding sequence and the Sm motif, U7 can no longer be involved in processing the histone pre-mRNA and instead targets pre-mRNAs or smRNA for blocking or splicing modulation. In this manner, U7 snRNA can be used as an effective gene therapy platform. The U7 snRNA platform also has the additional advantages of being a compact size, having the capability to accumulate in the nucleus without causing cellular toxicity, and possesses little to no immunoreactivity. (Gadgil et al., 2021, J Gene Med, 23(4): e3321.). The U7 snRNA platform is described in more detail in International Patent Application Publication No. WO 2023/168458, which is incorporated herein by reference in its entirety.

In some aspects disclosed herein are esnRNA comprising a stem loop (SL). Compensatory modifications made to the native stem loop sequence can create a SL which more effectively communicates (folds and anneals) with the snRNA interaction stabilization domain (ISD) compared to the native stem loop sequence, which in turn creates an snRNA platform with increased stability. U7 snRNAs have been previously shown to be programmable to modulate mRNAs. Disclosed herein are programmed engineered snRNA improvements which are capable of being used as a gene therapy tool.

snRNA systems disclosed herein are configured to bind target SCN1A or SYNGAP1 RNA sequences to modulate RNA splicing which can lead to single or multiple exon skipping or exon inclusion of targeted sequences of the SCN1A or SYNGAP1 RNA. SCN1A-targeting snRNA are configured to bind to SCN1A pre-mRNA molecules at sites that regulate RNA splicing. SYNGAP1-targeting snRNA are configured to bind to SYNGAP1 pre-mRNA molecules at sites that regulate RNA splicing. Splicing regulatory sites can include splice acceptor sequences, splice donor sequences, and exon splice enhancer sequences. snRNA sequences of the disclosure can induce exon skipping (of single or multiple exons) of targeted exonic sequences.

Unproductive splicing events such as inclusion of poison exons containing premature stop codons is a naturally occurring regulatory mechanism to reduce expression of unnecessary genes during development or in specific tissues. The SCN1A pre-mRNA contains a “poison exon” (exon 20N in humans, exon 21N in mice), which includes a premature termination codon. The SYNGAP1 pre-mRNA contains a “poison exon” (exon 11 in humans), which includes a premature termination codon. Transcription of the exon results in nonsense mediated decay of the RNA. Without wishing to be bound by theory, it is hypothesized that by promoting skipping the “poison exon” (Exon 20N) from the SCN1A pre-mRNA using the U7 snRNA platform, would increase expression of the full length SCN1A transcript and therefore promote expression of restored and functional Nav1.1 protein. Further without wishing to be bound by theory, it is hypothesized that by promoting skipping the “poison exon” (Exon 11) from the SYNGAP1 pre-mRNA using the U7 snRNA platform, would increase expression of the full length SYNGAP1 transcript and therefore promote expression of restored and functional SYNGAP1 protein.

In one embodiment, these snRNAs are human snRNAs. In another embodiment, these snRNAs are mouse snRNAs. In another embodiment, the snRNAs are of any species. In another embodiment, the snRNAs are a combination of human and mouse snRNAs. In one embodiment, the U7 snRNA is a human U7 snRNA or a mouse U7 snRNA. In another embodiment disclosed herein, snRNA comprises varying types of snRNAs (U1-U12, etc.) by combining domains of endogenous snRNAs to fine tune stabilization of the platform and/or to reduce off-target effects. For example, in one embodiment, the snRNA system comprises a combination of human or mouse U7 snRNA and human or mouse U1 snRNA components.

Additional elements that can tune the processing and abundance of the RNA can be further engineered into the snRNAs or esnRNAs comprising SLs. In one embodiment, additional elements that can tune the processing, stability, and abundance of the esnRNA can be further engineered into the esnRNAs at the 5′ or 3′ ends. In another embodiment, such elements may include but are not limited to stem loops, hairpins, G-C clamps, kissing loops, triplexes, quadruplexes, and protein binding sites.

The snRNA platform and portions thereof can be used in a therapeutic setting and context so long as a suitable spacer(s) or target sequence (s) TS(s) is included in the design of the therapeutic composition. In certain embodiments, a therapeutic snRNA composition is used to treat a disease associated with dysregulated, mutated, or non-functional NaV1.1. In some aspects, the disease or disorder is Dravet Syndrome

### Targeting Sequences

The snRNA systems can be programmed to comprise a targeting sequence (TS) (also termed “spacer”) that targets an RNA of interest. The snRNA systems can be programmed with one or more targeting sequences targeting one or more RNAs of interest. In some aspects, the targeting sequence is a 5′ targeting sequence (5′TS) that targets one or more RNAs of interest. In this context, 5′ is in reference to the snRNA insert's 5′ end and not necessarily to the overall vector configuration comprising the snRNA insert or inserts. The TS can be located in or near the 5′ end of the snRNA. In an alternative embodiment, the targeting sequence(s) (TS) can be located in or near a 3′ position in the snRNA construct, thereby generating a 3′ targeting sequence (3′ TS), particularly if the snRNA construct is not a U7-based snRNA.

Targeting sequences of the disclosure, including 5′ TS, and 3′TS can be between about 1 and about 200 nucleotides in length. In some aspects, targeting sequences of the disclosure are between about 10 and about 150 nucleotides in length. In some aspects, targeting sequences of the disclosure are between about 10 and about 100 nucleotides in length. In some aspects, targeting sequences of the disclosure are between about 20 and about 60 nucleotides in length. In some aspects, targeting sequences of the disclosure are at least about 10, 20, 30, 40, 50, 60, or about 70 nucleotides in length.

snRNA compositions of the disclosure can comprise more than one targeting sequence, wherein each targeting sequence binds a distinct RNA sequence. In some aspects, snRNA of the disclosure comprise a fusion targeting sequence. In some aspects, a fusion targeting sequence is a nucleic acid sequence comprising two targeting sequences directly connected or connected by one or more linker nucleic acid sequences, wherein each targeting sequence binds a different target RNA sequence.

In one example, U7 snRNA can be programmed by replacing the histone mRNA binding sequence with a sequence complementary to a target of interest. In some aspects snRNA systems of the disclosure bind a target mRNA or pre-mRNA sequence of interest. The exemplary snRNA systems shown herein lead to exon skipping (for treating Dravet Syndrome, e.g., SCN1A exon 20N skipping; or for treating SYNGAP1-related epilepsy, e.g. SYNGAP1 exon 11 skipping).

In some embodiments, snRNAs of the disclosure target a pre-mRNA or mRNA sequence encoding the NaV1.1 protein. SCN1A is a gene encoding the protein NaV1.1. Mutations in SCN1A are associated with Dravet Syndrome. In some embodiments, the SCN1A RNA sequence targeted by snRNA compositions of the disclosure can be any exonic or intronic SCN1A RNA sequence. In some embodiments, the SCN1A RNA sequence targeted by snRNA compositions of the disclosure is an exon 20N or Exon 21N SCN1A RNA sequence. In some embodiments, the SCN1A RNA sequence targeted by snRNA compositions of the disclosure is a human exon 20N SCN1A RNA sequence. In some embodiments, the SCN1A RNA sequence targeted by snRNA compositions of the disclosure is a murine exon 21N SCN1A RNA sequence. In some embodiments, the SCN1A RNA sequence targeted by the snRNA of the disclosure is a splice acceptor sequence, a splice donor sequence, or an exon splice enhancer sequence.

In some embodiments, the nucleic acid sequence encoding wild-type human SCN1A mRNA comprises or consists of SEQ ID NO: 1. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 1.

In some embodiments, the nucleic acid sequence encoding wild-type human SCN1A mRNA comprises or consists of SEQ ID NO: 2. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 2.

In some embodiments, the nucleic acid sequence encoding wild-type human SCN1A mRNA comprises or consists of SEQ ID NO: 3. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 3.

In some embodiments, the nucleic acid sequence encoding wild-type human SCN1A mRNA comprises or consists of SEQ ID NO: 4. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 4.

In some embodiments, the nucleic acid sequence encoding wild-type human SCN1A mRNA comprises or consists of SEQ ID NO: 5. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 5.

In some embodiments, the nucleic acid sequence encoding wild-type human SCN1A mRNA comprises or consists of SEQ ID NO: 6. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 6.

A comprises or consists of SEQ ID NO: 7. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 7.

In some embodiments, the nucleic acid sequence encoding wild-type human SCN1A mRNA comprises or consists of SEQ ID NO: 8. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 8.

In some embodiments, the nucleic acid sequence encoding wild-type human SCN1A mRNA comprises or consists of SEQ ID NO: 9. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 9.

In some embodiments, the nucleic acid sequence encoding wild-type human SCN1A mRNA comprises or consists of SEQ ID NO: 10. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 10.

In some embodiments, the nucleic acid sequence encoding wild-type human SCN1A mRNA comprises or consists of SEQ ID NO: 11. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 11.

In some embodiments, the nucleic acid sequence encoding wild-type human SCN1A mRNA comprises or consists of SEQ ID NO: 12. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 12.

In some embodiments, the nucleic acid sequence encoding wild-type human SCN1A mRNA comprises or consists of SEQ ID NO: 13. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 13.

In some embodiments, the nucleic acid sequence encoding wild-type human SCN1A mRNA comprises or consists of SEQ ID NO: 14. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 14.

In some embodiments, the nucleic acid sequence encoding wild-type human SCN1A mRNA comprises or consists of SEQ ID NO: 15. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 15.

Target sequence that binds SCN1A exon 20N can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 1, which follows:

TABLE 1
Exon 20N Targeting Sequences (Single Spacers)

| Plasmid UID | Spacer | Dravet single spacers | SEQ ID NO |
|---|---|---|---|
| P04169 | z1 | AATAAAGGGCTCAGGGGAGGAACCAG | 16 |
| P04170 | z2 | AAAGGGCTCAGGGGAGGAACCAGCGC | 17 |
| P04171 | z3 | GGGCTCAGGGGAGGAACCAGCGCTCC | 18 |
| P04172 | z4 | TCAGGGGAGGAACCAGCGCTCCACCC | 19 |
| P04173 | z5 | CCATAATAAAGGGCTCAGGGGAGGAA | 20 |
| P04706 | z6 | ccaagttggagcaagattatcctatac | 21 |
| P04707 | z7 | gtacccataataaagggctcaggggaggaa | 22 |
| P04708 | z8 | gtacccataataaagggctcagggga | 23 |
| P04709 | z9 | ggtagcaaaaggggtaatacagtaccc | 24 |
| P04710 | z10 | ccccatccaagttggagcaagattatcc | 25 |
| P04754 | z11 | gttggagcaagattatccta | 26 |
| P04755 | z12 | aaaggggtaatacagtaccc | 27 |
| P04756 | z13 | cccataataaagggctcagg | 28 |
| P04757 | z14 | aataaagggctcaggggagg | 29 |
| P05074 | z15 | aggggtaatacagtaccc | 30 |
| P05079 | z16 | aaggattaaaggtagcaa | 31 |
| P05080 | z17 | cataagtcacagtgcaag | 32 |
| P05081 | z18 | ccactacacataagtcacagtgcaag | 33 |
| P05082 | z19 | tacacataagtcacagtgcaaggatt | 34 |
| P05083 | z20 | cataagtcacagtgcaaggattaaag | 35 |
| P05084 | z21 | agtcacagtgcaaggattaaaggtag | 36 |
| P05085 | z22 | acagtgcaaggattaaaggtagcaaa | 37 |
| P05086 | z23 | tgcaaggattaaaggtagcaaaaggg | 38 |
| P05087 | z24 | aggattaaaggtagcaaaaggggtaa | 39 |
| P05088 | z25 | ttaaaggtagcaaaaggggtaataca | 40 |
| P05089 | z26 | aggtagcaaaaggggtaatacagtac | 41 |
| P05090 | z27 | agcaaaaggggtaatacagtacccat | 42 |
| P05091 | z28 | aaaggggtaatacagtacccataata | 43 |
| P05092 | z29 | gggtaatacagtacccataataaagg | 44 |
| P05093 | z30 | gctccaccccatccaagttggagcaa | 45 |
| P05094 | z31 | ccagcgctccaccccatccaagttgg | 46 |
| P05095 | z32 | aggaaccagcgctccaccccatccaa | 47 |
| P05166 | z33 | AGTTGGAGCAAGATTATCCTA | 48 |
| P05167 | z34 | CCAAGTTGGAGCAAGATTATC | 49 |
| P05168 | z35 | AGTTGGAGCAAGATTATC | 50 |
| P05314 | z36 | AGTACCCATAATAAAGGG | 51 |

In some embodiments, the target sequence that binds SCN1A exon 20N comprises the sequence set forth in SEQ ID NO: 16. In some embodiments, the target sequence that binds SCN1A exon 20N comprises the sequence set forth in SEQ ID NO: 17. In some embodiments, the target sequence that binds SCN1A exon 20N comprises the sequence set forth in SEQ ID NO: 18. In some embodiments, the target sequence that binds SCN1A exon 20N comprises the sequence set forth in SEQ ID NO: 19. In some embodiments, the target sequence that binds SCN1A exon 20N comprises the sequence set forth in SEQ ID NO: 20. In some embodiments, the target sequence that binds SCN1A exon 20N comprises the sequence set forth in SEQ ID NO: 21. In some embodiments, the target sequence that binds SCN1A exon 20N comprises the sequence set forth in SEQ ID NO: 22. In some embodiments, the target sequence that binds SCN1A exon 20N comprises the sequence set forth in SEQ ID NO: 23. In some embodiments, the target sequence that binds SCN1A exon 20N comprises the sequence set forth in SEQ ID NO: 24. In some embodiments, the target sequence that binds SCN1A exon 20N comprises the sequence set forth in SEQ ID NO: 25. In some embodiments, the target sequence that binds SCN1A exon 20N comprises the sequence set forth in SEQ ID NO: 26. In some embodiments, the target sequence that binds SCN1A exon 20N comprises the sequence set forth in SEQ ID NO: 27. In some embodiments, the target sequence that binds SCN1A exon 20N comprises the sequence set forth in SEQ ID NO: 28. In some embodiments, the target sequence that binds SCN1A exon 20N comprises the sequence set forth in SEQ ID NO: 29. In some embodiments, the target sequence that binds SCN1A exon 20N comprises the sequence set forth in SEQ ID NO: 30. In some embodiments, the target sequence that binds SCN1A exon 20N comprises the sequence set forth in SEQ ID NO: 31. In some embodiments, the target sequence that binds SCN1A exon 20N comprises the sequence set forth in SEQ ID NO: 32. In some embodiments, the target sequence that binds SCN1A exon 20N comprises the sequence set forth in SEQ ID NO: 33. In some embodiments, the target sequence that binds SCN1A exon 20N comprises the sequence set forth in SEQ ID NO: 34. In some embodiments, the target sequence that binds SCN1A exon 20N comprises the sequence set forth in SEQ ID NO: 35. In some embodiments, the target sequence that binds SCN1A exon 20N comprises the sequence set forth in SEQ ID NO: 36. In some embodiments, the target sequence that binds SCN1A exon 20N comprises the sequence set forth in SEQ ID NO: 37. In some embodiments, the target sequence that binds SCN1A exon 20N comprises the sequence set forth in SEQ ID NO: 38. In some embodiments, the target sequence that binds SCN1A exon 20N comprises the sequence set forth in SEQ ID NO: 39. In some embodiments, the target sequence that binds SCN1A exon 20N comprises the sequence set forth in SEQ ID NO: 40. In some embodiments, the target sequence that binds SCN1A exon 20N comprises the sequence set forth in SEQ ID NO: 41. In some embodiments, the target sequence that binds SCN1A exon 20N comprises the sequence set forth in SEQ ID NO: 42. In some embodiments, the target sequence that binds SCN1A exon 20N comprises the sequence set forth in SEQ ID NO: 43. In some embodiments, the target sequence that binds SCN1A exon 20N comprises the sequence set forth in SEQ ID NO: 44. In some embodiments, the target sequence that binds SCN1A exon 20N comprises the sequence set forth in SEQ ID NO: 45. In some embodiments, the target sequence that binds SCN1A exon 20N comprises the sequence set forth in SEQ ID NO: 46. In some embodiments, the target sequence that binds SCN1A exon 20N comprises the sequence set forth in SEQ ID NO: 47. In some embodiments, the target sequence that binds SCN1A exon 20N comprises the sequence set forth in SEQ ID NO: 48. In some embodiments, the target sequence that binds SCN1A exon 20N comprises the sequence set forth in SEQ ID NO: 49. In some embodiments, the target sequence that binds SCN1A exon 20N comprises the sequence set forth in SEQ ID NO: 50. In some embodiments, the target sequence that binds SCN1A exon 20N comprises the sequence set forth in SEQ ID NO: 51.

The sequences set forth in Table may be combined to generate fusion spaces. Any first sequence set forth in Table 1 may combined with any second sequence set forth in in Table. Illustrative fusion spacers are set forth in Table 2. Target sequence that binds SCN1A exon 20N can comprise, consist essentially of, or consist of a nucleic acid sequence at least 650%, 700%, 7500, 8000, 8500, 9000, 95%, 960%, 97%, 980%, 9900 or 1000% (or any percentage in between) identical to one or more of the following nucleotide sequences set forth in Table, which follow.

TABLE 2
Exon 20N Targeting Sequences (Fusion Spacers)

| Plasmid | | | SEQ |
|---|---|---|---|
| UID | Spacer | Dravet Fusion spacers | ID NO |
| P05075 | z12/z11 | aaaggggtaatacagtacccgttggagcaagattatccta | 52 |
| P05076 | z15/z11 | aggggtaatacagtacccgttggagcaagattatccta | 53 |
| A05378 | z12/z6 | aaaggggtaatacagtacccccaagttggagcaagattatcctatac | 54 |
| A05290 | z15/z6 | aggggtaatacagtacccccaagttggagcaagattatcctatac | 55 |
| P05162 | z11/z12 | gttggagcaagattatcctaaaaggggtaatacagtaccc | 56 |
| A05291 | z11/z15 | gttggagcaagattatcctaaggggtaatacagtaccc | 57 |
| P05164 | z6/z12 | ccaagttggagcaagattatcctatacaaaggggtaatacagtaccc | 58 |
| P05165 | z6/z15 | ccaagttggagcaagattatcctatacaggggtaatacagtaccc | 59 |
| P05292 | z6/z22 | ccaagttggagcaagattatcctatacacagtgcaaggattaaaggtagca | 60 |
| | | aa | |
| P05293 | z22/z6 | acagtgcaaggattaaaggtagcaaaccaagttggagcaagattatcctat | 61 |
| | | ac | |
| P05294 | z6/z24 | ccaagttggagcaagattatcctatacaggattaaaggtagcaaaagggg | 62 |
| | | taa | |
| P05295 | z24/z6 | aggattaaaggtagcaaaaggggtaaccaagttggagcaagattatccta | 63 |
| | | tac | |
| P05296 | z6/z36 | ccaagttggagcaagattatcctatacAGTACCCATAATAAA | 64 |
| | | GGG | |
| P05297 | z36/z6 | AGTACCCATAATAAAGGGccaagttggagcaagattatc | 65 |
| | | ctatac | |
| P05298 | z32/z15 | AGGAACCAGCGCTCCACCCCATCCAAaggggtaa | 66 |
| | | tacagtaccc | |
| P05299 | z15/z32 | aggggtaatacagtacccAGGAACCAGCGCTCCACCCC | 67 |
| | | ATCCAA | |
| P05300 | z32/z22 | AGGAACCAGCGCTCCACCCCATCCAAacagtgca | 68 |
| | | aggattaaaggtagcaaa | |
| P05301 | z22/z32 | acagtgcaaggattaaaggtagcaaaAGGAACCAGCGCTC | 69 |
| | | CACCCCATCCAA | |
| P05302 | z32/z24 | AGGAACCAGCGCTCCACCCCATCCAAaggattaa | 70 |
| | | aggtagcaaaaggggtaa | |
| P05303 | z24/z32 | aggattaaaggtagcaaaaggggtaaAGGAACCAGCGCTC | 71 |
| | | CACCCCATCCAA | |
| P05304 | z32/z36 | AGGAACCAGCGCTCCACCCCATCCAAAGTAC | 72 |
| | | CCATAATAAAGGG | |
| P05305 | z36/z32 | AGTACCCATAATAAAGGGAGGAACCAGCGCT | 73 |
| | | CCACCCCATCCAA | |
| A05379 | z33/z15 | AGTTGGAGCAAGATTATCCTAaggggtaatacagtac | 74 |
| | | CC | |
| A05380 | z15/z33 | aggggtaatacagtacccAGTTGGAGCAAGATTATCCT | 75 |
| | | A | |
| P05308 | z33/z22 | AGTTGGAGCAAGATTATCCTAacagtgcaaggattaa | 76 |
| | | aggtagcaaa | |
| P05309 | z22/z33 | acagtgcaaggattaaaggtagcaaaAGTTGGAGCAAGAT | 77 |
| | | TATCCTA | |
| P05310 | z33/z24 | AGTTGGAGCAAGATTATCCTAaggattaaaggtagca | 78 |
| | | aaaggggtaa | |
| P05311 | z24/z33 | aggattaaaggtagcaaaaggggtaaAGTTGGAGCAAGAT | 79 |
| | | TATCCTA | |
| A05381 | z33/z36 | AGTTGGAGCAAGATTATCCTAAGTACCCATA | 80 |
| | | ATAAAGGG | |
| A05382 | z36/z33 | AGTACCCATAATAAAGGGAGTTGGAGCAAG | 81 |
| | | ATTATCCTA | |
| A05383 | z21/z33 | agtcacagtgcaaggattaaaggtagAGTTGGAGCAAGATT | 82 |
| | | ATCCTA | |
| A05384 | z33/z21 | AGTTGGAGCAAGATTATCCTAagtcacagtgcaagga | 83 |
| | | ttaaaggtag | |

In some embodiments, the target sequence that binds SCN1A exon 20N comprises the sequence set forth in SEQ ID NO: 52. In some embodiments, the target sequence that binds SCN1A exon 20N comprises the sequence set forth in SEQ ID NO: 53. In some embodiments, the target sequence that binds SCN1A exon 20N comprises the sequence set forth in SEQ ID NO: 54. In some embodiments, the target sequence that binds SCN1A exon 20N comprises the sequence set forth in SEQ ID NO: 55. In some embodiments, the target sequence that binds SCN1A exon 20N comprises the sequence set forth in SEQ ID NO: 56. In some embodiments, the target sequence that binds SCN1A exon 20N comprises the sequence set forth in SEQ ID NO: 57. In some embodiments, the target sequence that binds SCN1A exon 20N comprises the sequence set forth in SEQ ID NO: 58. In some embodiments, the target sequence that binds SCN1A exon 20N comprises the sequence set forth in SEQ ID NO: 59. In some embodiments, the target sequence that binds SCN1A exon 20N comprises the sequence set forth in SEQ ID NO: 60. In some embodiments, the target sequence that binds SCN1A exon 20N comprises the sequence set forth in SEQ ID NO: 61. In some embodiments, the target sequence that binds SCN1A exon 20N comprises the sequence set forth in SEQ ID NO: 62. In some embodiments, the target sequence that binds SCN1A exon 20N comprises the sequence set forth in SEQ ID NO: 63. In some embodiments, the target sequence that binds SCN1A exon 20N comprises the sequence set forth in SEQ ID NO: 64. In some embodiments, the target sequence that binds SCN1A exon 20N comprises the sequence set forth in SEQ ID NO: 65. In some embodiments, the target sequence that binds SCN1A exon 20N comprises the sequence set forth in SEQ ID NO: 66. In some embodiments, the target sequence that binds SCN1A exon 20N comprises the sequence set forth in SEQ ID NO: 67. In some embodiments, the target sequence that binds SCN1A exon 20N comprises the sequence set forth in SEQ ID NO: 68. In some embodiments, the target sequence that binds SCN1A exon 20N comprises the sequence set forth in SEQ ID NO: 69. In some embodiments, the target sequence that binds SCN1A exon 20N comprises the sequence set forth in SEQ ID NO: 70. In some embodiments, the target sequence that binds SCN1A exon 20N comprises the sequence set forth in SEQ ID NO: 71. In some embodiments, the target sequence that binds SCN1A exon 20N comprises the sequence set forth in SEQ ID NO: 72. In some embodiments, the target sequence that binds SCN1A exon 20N comprises the sequence set forth in SEQ ID NO: 73. In some embodiments, the target sequence that binds SCN1A exon 20N comprises the sequence set forth in SEQ ID NO: 74. In some embodiments, the target sequence that binds SCN1A exon 20N comprises the sequence set forth in SEQ ID NO: 75. In some embodiments, the target sequence that binds SCN1A exon 20N comprises the sequence set forth in SEQ ID NO: 76. In some embodiments, the target sequence that binds SCN1A exon 20N comprises the sequence set forth in SEQ ID NO: 77. In some embodiments, the target sequence that binds SCN1A exon 20N comprises the sequence set forth in SEQ ID NO: 78. In some embodiments, the target sequence that binds SCN1A exon 20N comprises the sequence set forth in SEQ ID NO: 79. In some embodiments, the target sequence that binds SCN1A exon 20N comprises the sequence set forth in SEQ ID NO: 80. In some embodiments, the target sequence that binds SCN1A exon 20N comprises the sequence set forth in SEQ ID NO: 81. In some embodiments, the target sequence that binds SCN1A exon 20N comprises the sequence set forth in SEQ ID NO: 82. In some embodiments, the target sequence that binds SCN1A exon 20N comprises the sequence set forth in SEQ ID NO: 83.

In some embodiments, snRNAs of the disclosure target a pre-mRNA or mRNA sequence encoding the Synaptic Ras GTPase-activating protein 1 protein. SYNGAP1 is a gene encoding the protein Synaptic Ras GTPase-activating protein 1 (SYNGAP1). Mutations in SYNGAP1 are associated with SYNGAP1-related epilepsy. In some embodiments, the SYNGAP1 RNA sequence targeted by snRNA compositions of the disclosure can be any exonic or intronic SYNGAP1 RNA sequence. In some embodiments, the SYNGAP1 RNA sequence targeted by snRNA compositions of the disclosure is an exon 11 or poison exon 11 SYNGAP1 RNA sequence. In some embodiments, the SYNGAP1 RNA sequence targeted by snRNA compositions of the disclosure is a human exon 11 or poison exon 11 SYNGAP1 RNA sequence. In some embodiments, the SYNGAP1 RNA sequence targeted by snRNA compositions of the disclosure is a murine exon 11 or poison exon 11 SCN1A RNA sequence. In some embodiments, the SYNGAP1 RNA sequence targeted by the snRNA of the disclosure is a splice acceptor sequence, a splice donor sequence, or an exon splice enhancer sequence.

In some embodiments, the nucleic acid sequence encoding wild-type human SYNGAP1 mRNA comprises or consists of SEQ ID NO: 280. In some embodiments, the nucleic acid sequence encoding wild-type human SCN1A mRNA comprises a sequence that is at least 65%, 70%, 75%, 80%, 85%, 90%, 95%, 96%, 97%, 98%, or 99% identical to the sequence set forth in SEQ ID NO: 280.

The sequences set forth in Table 3 may be combined to generate fusion spaces. Any first sequence set forth in Table 3 may combine with any second sequence set forth in Table 3. Illustrative fusion spacers are set forth in Table 3. Target sequences that binds SYNGAP1 exon 11 or poison exon 11 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 3, which follow.

TABLE 3
Exon 20N Targeting Sequences and Exon 20N Fusion Targeting Sequences

| Plasmid | | | |
|---|---|---|---|
| UID | Spacer | SYNGAP1 spacers | SEQ ID NO |
| P05326 | z1 | AAAAACAGACACCAGGGAGAGACAGAGATG | 181 |
| P05327 | z2 | GACACCAGGGAGAGACAG | 182 |
| P05328 | z3 | AGAAAAACAGACACCAGGGAGAGACAGAGA | 183 |
| P05329 | z4 | AGAAGAAAAACAGACACCAGGGAGAGACAG | 184 |
| P05330 | z5 | AAACAGACACCAGGGAGAGACAGAG | 185 |
| P05331 | z6 | GGGAGAGACACGTGGGAGAGAGATGGAGGG | 186 |
| P05332 | z7 | AGGGGAGAGACACGTGGGAGAGAGATGGAG | 187 |
| P05333 | z8 | GAGGGGAGAGACACGTGGGAGAGAGATGGA | 188 |
| P05334 | z9 | TGAGGGGAGAGACACGTGGGAGAGAGATGG | 189 |
| P05335 | z10 | GTGAGGGGAGAGACACGTGGGAGAGAGATG | 190 |
| P05336 | z11 | GGTGAGGGGAGAGACACGTGGGAGAGAGAT | 191 |
| P05337 | z12 | GAGAGACACGTGGGAGAGAGATGGAGGGGT | 192 |
| P05338 | z13 | AAGGTGAGGGGAGAGACACGTGGGAGAGAG | 193 |
| P05339 | z14 | GAGAGACACGTGGGAGAGAGATGG | 194 |
| P05340 | z15 | AGGGGAGAGACACGTGGGAGAGAGATGG | 195 |
| P05341 | z16 | GGAGAGACACGTGGGAGAGAGATGG | 196 |
| P05342 | z17 | GACACGTGGGAGAGAGATGG | 197 |
| P05343 | z18 | GACCCTCAGCTTCCAGGGAACATGCTGAGG | 198 |
| P05344 | z19 | CTCAGCTTCCAGGGAACATGCTGAGGGGGG | 199 |
| P05345 | z20 | CCCTCAGCTTCCAGGGAACATGCTGAGGGG | 200 |
| P05346 | z21 | GAGACCCTCAGCTTCCAGGGAACATGCTGA | 201 |
| P05347 | z22 | GCTTCCAGGGAACATGCTGAGGGG | 202 |
| P05348 | z23/ET- | UCCAGGGAACAUGCUGAG | 203 |
| | 085/ | | |
| | P05348 | | |
| P05349 | z24 | CAGCTTCCAGGGAACATGCTGAGGGGGGTG | 204 |
| P05350 | z25 | GAGAGAAAGAGAGAGACCGGGACTGAGCCC | 205 |
| P05351 | z1 + z12 | AAAAACAGACACCAGGGAGAGACAGAGATG | 206 |
| | | GAGAGACACGTGGGAGAGAGATGGAGGGGT | |
| P05352 | z1 + z8 | AAAAACAGACACCAGGGAGAGACAGAGATG | 207 |
| | | GAGGGGAGAGACACGTGGGAGAGAGATGGA | |
| P05353 | z12 + z24 | GAGAGACACGTGGGAGAGAGATGGAGGGGT | 208 |
| | | CAGCTTCCAGGGAACATGCTGAGGGGGGTG | |
| P05354 | z12 + z20 | GAGAGACACGTGGGAGAGAGATGGAGGGGT | 209 |
| | | CCCTCAGCTTCCAGGGAACATGCTGAGGGG | |
| P05355 | z8 + z24 | GAGGGGAGAGACACGTGGGAGAGAGATGGA | 210 |
| | | CAGCTTCCAGGGAACATGCTGAGGGGGGTG | |
| P05356 | z8 + z20 | GAGGGGAGAGACACGTGGGAGAGAGATGGA | 211 |
| | | CCCTCAGCTTCCAGGGAACATGCTGAGGGG | |
| P05357 | z24 + z12 | CAGCTTCCAGGGAACATGCTGAGGGGGGTGG | 212 |
| | | AGAGACACGTGGGAGAGAGATGGAGGGGT | |
| P05358 | z20 + z12 | CCCTCAGCTTCCAGGGAACATGCTGAGGGGG | 213 |
| | | AGAGACACGTGGGAGAGAGATGGAGGGGT | |
| P05359 | z24 + z8 | CAGCTTCCAGGGAACATGCTGAGGGGGGTGG | 214 |
| | | AGGGGAGAGACACGTGGGAGAGAGATGGA | |
| P05360 | z20 + z8 | CCCTCAGCTTCCAGGGAACATGCTGAGGGGG | 215 |
| | | AGGGGAGAGACACGTGGGAGAGAGATGGA | |
| P05361 | z1 + 20 | AAAAACAGACACCAGGGAGAGACAGAGATG | 216 |
| | | CCCTCAGCTTCCAGGGAACATGCTGAGGGG | |
| P05450 | z26 | CACGTGGGAGAGAGATGG | 217 |
| P05451 | z27 | CCCTCAGCTTCCAGGGAACATGCTGAGGGGG | 218 |
| | | GTGGT | |
| P05452 | z28 | GACCCTCAGCTTCCAGGGAACATGCTGAGGG | 219 |
| | | GGGTGGTAG | |
| P05453 | z29 | GCTTCCAGGGAACATGCTGAGGGGGGTGGTA | 220 |
| | | GGAGGTGAG | |
| P05454 | z30 | AAGAGAGAGACCGGGACTGAGCCCCAGAGA | 221 |
| | | CCCTCAGCTTCCAGGGAACATGCTGAGGGG | |
| P05455 | z31 | GCTTCCAGGGAACATGCTGAGGGGGGTGGT | 222 |
| P05456 | z32 | TCCAGGGAACATGCTGAGGGGGGTGGTAGG | 223 |
| P05457 | z33 | GAGAGAGACCGGGACUGA | 224 |
| P05458 | z34 | GAGAAAGAGAGAGACCGGGACTGAGCCCCA | 225 |
| P05459 | z35 | GGACCGCAGGAGCAAGATGGGAGGCTGCTT | 226 |
| P05460 | z36 | GGGACCGCAGGAGCAAGATGGGAGGCTGCT | 227 |
| P05461 | z37 | AGGGACCGCAGGAGCAAGATGGGAGGCTGC | 228 |
| P05462 | z38 | GAGGGACCGCAGGAGCAAGATGGGAGGCTG | 229 |
| P05463 | z39 | GGAGGGACCGCAGGAGCAAGATGGGAGGCT | 230 |
| P05464 | z40 | GGACCGCAGGAGCAAGATGGGAGGCTGCTT | 231 |
| | | GAAGA | |
| P05465 | z41 | GCAGGAGCAAGATGGGAGGCTGCTT | 232 |
| P05466 | z42 | GAGACACGTGGGAGAGAG | 233 |
| P05467 | z43 | AGACACGTGGGAGAGAGATGGAGG | 234 |
| P05468 | z44 | AGGTGAGGGGAGAGACACGTGGGAGAGAGA | 235 |
| | | TGGAGGGGTG | |
| P05469 | z45 | AGGGGAGAGACACGTGGGAGAGAG | 236 |
| P05470 | z46 | CACGTGGGAGAGAGATGGAGGGGT | 237 |
| P05471 | z22 + z25 | GCTTCCAGGGAACATGCTGAGGGGGAGAGA | 238 |
| | | AAGAGAGAGACCGGGACTGAGCCC | |
| P05472 | z23 + z25 | TCCAGGGAACATGCTGAGGAGAGAAAGAGA | 239 |
| | | GAGACCGGGACTGAGCCC | |
| P05473 | z24 + z25 | CAGCTTCCAGGGAACATGCTGAGGGGGGTGG | 240 |
| | | AGAGAAAGAGAGAGACCGGGACTGAGCCC | |
| P05474 | z25 + z22 | GAGAGAAAGAGAGAGACCGGGACTGAGCCC | 241 |
| | | GCTTCCAGGGAACATGCTGAGGGG | |
| P05475 | z25 + z23 | GAGAGAAAGAGAGAGACCGGGACTGAGCCC | 242 |
| | | TCCAGGGAACATGCTGAG | |
| P05476 | z25 + z24 | GAGAGAAAGAGAGAGACCGGGACTGAGCCC | 243 |
| | | CAGCTTCCAGGGAACATGCTGAGGGGGGTG | |
| P05477 | z14 + z24 | GAGAGACACGTGGGAGAGAGATGGCAGCTT | 244 |
| | | CCAGGGAACATGCTGAGGGGGGTG | |
| P05478 | z24 + z14 | CAGCTTCCAGGGAACATGCTGAGGGGGGTGG | 245 |
| | | AGAGACACGTGGGAGAGAGATGG | |
| P05479 | z16 + z24 | GGAGAGACACGTGGGAGAGAGATGGCAGCT | 246 |
| | | TCCAGGGAACATGCTGAGGGGGGTG | |
| P05480 | z16 + z23 | GGAGAGACACGTGGGAGAGAGATGGTCCAG | 247 |
| | | GGAACATGCTGAG | |
| P05481 | z24 + z16 | CAGCTTCCAGGGAACATGCTGAGGGGGGGG | 248 |
| | | GAGAGACACGTGGGAGAGAGATGG | |
| P05482 | z23 + z16 | TCCAGGGAACATGCTGAGGGAGAGACACGT | 249 |
| | | GGGAGAGAGATGG | |
| P05483 | z23 + z23 | TCCAGGGAACATGCTGAGTCCAGGGAACATG | 250 |
| | | CTGAG | |
| | z2, 20 | AGACACCAGGGAGAGACAGA | 251 |
| | nt | | |
| | z2, 19 | CAGACACCAGGGAGAGACA | 252 |
| | nt | | |
| | z26, 19 | ACACGUGGGAGAGAGAUGG | 253 |
| | nt | | |
| | (z26a) | | |
| | z26, 20 | ACACGUGGGAGAGAGAUGGA | 254 |
| | nt | | |
| | (z26b) | | |
| | z26, 22 | GAGACACGUGGGAGAGAGAUGG | 255 |
| | nt | | |
| | (z26c) | | |
| | z47 | GAGAGAGACCGGGACUGAG | 256 |
| | z48 | GGGGGGAAGGGUCGGGGA | 257 |
| | z49 | CUGGGGGGAAGGGUCGGGGA | 258 |
| | z50 | CGGGAACACGCUGGGGGGAAGGGUCGGGGA | 259 |
| | z51 | ACCUCCUUCAGCUCCCUCGGGAACACGCUG | 260 |
| | z23/z47 | UCCAGGGAACAUGCUGAGGAGAGAGACCGG | 261 |
| | | GACUGAG | |
| | z47/z23 | GAGAGAGACCGGGACUGAGUCCAGGGAACA | 262 |
| | | UGCUGAG | |
| | z24/z47 | CAGCUUCCAGGGAACAUGCUGAGGGGGGUG | 263 |
| | | GAGAGAGACCGGGACUGAG | |
| | z47/z24 | GAGAGAGACCGGGACUGAGCAGCUUCCAGG | 264 |
| | | GAACAUGCUGAGGGGGGUG | |
| | z14/z24 | GAGAGACACGUGGGAGAGAGAUGGCAGCUU | 265 |
| | | CCAGGGAACAUGCUGAGGGGGGUG | |
| | z49/z24 | CUGGGGGGAAGGGUCGGGGACAGCUUCCAG | 266 |
| | | GGAACAUGCUGAGGGGGGUG | |
| L06064 | z49/z23 | CUGGGGGGAAGGGUCGGGGAUCCAGGGAAC | 267 |
| | | AUGCUGAG | |
| | z23/z49 | UCCAGGGAACAUGCUGAGCUGGGGGGAAGG | 268 |
| | | GUCGGGGA | |
| | z24/z49 | CAGCUUCCAGGGAACAUGCUGAGGGGGGUG | 269 |
| | | CUGGGGGGAAGGGUCGGGGA | |
| | z50/z24 | CGGGAACACGCUGGGGGGAAGGGUCGGGGA | 270 |
| | | CAGCUUCCAGGGAACAUGCUGAGGGGGGUG | |
| L06068 | z50/z23 | CGGGAACACGCUGGGGGGAAGGGUCGGGGA | 271 |
| | | UCCAGGGAACAUGCUGAG | |
| | z47/z49 | GAGAGAGACCGGGACUGAGCUGGGGGGAAG | 272 |
| | | GGUCGGGGA | |
| | z49/z47 | CUGGGGGGAAGGGUCGGGGAGAGAGAGACC | 273 |
| | | GGGACUGAG | |
| | z24/z50 | CUGGGGGGAAGGGUCGGGGACGGGAACACG | 274 |
| | | CUGGGGGGAAGGGUCGGGGA | |
| | z14/z49 | GAGAGACACGUGGGAGAGAGAUGGCUGGGG | 275 |
| | | GGAAGGGUCGGGGA | |
| | z49/z14 | CUGGGGGGAAGGGUCGGGGAGAGAGACACG | 276 |
| | | UGGGAGAGAGAUGG | |
| | z23/z14 | UCCAGGGAACAUGCUGAGGAGAGACACGUG | 277 |
| | | GGAGAGAGAUGG | |
| | z14/z23 | GAGAGACACGUGGGAGAGAGAUGGUCCAGG | 278 |
| | | GAACAUGCUGAG | |
| | z49/z26 | CUGGGGGGAAGGGUCGGGGACACGUGGGAG | 279 |
| | | AGAGAUGG | |

In some embodiments, the target sequence that binds SYNGAP1 exon 11 or poison exon 11 comprises the sequence set forth in SEQ ID NO: 181. In some embodiments, the target sequence that binds SYNGAP1 exon 11 or poison exon 11 comprises the sequence set forth in SEQ ID NO: 182. In some embodiments, the target sequence that binds SYNGAP1 exon 11 or poison exon 11 comprises the sequence set forth in SEQ ID NO: 183. In some embodiments, the target sequence that binds SYNGAP1 exon 11 or poison exon 11 comprises the sequence set forth in SEQ ID NO: 184. In some embodiments, the target sequence that binds SYNGAP1 exon 11 or poison exon 11 comprises the sequence set forth in SEQ ID NO: 185. In some embodiments, the target sequence that binds SYNGAP1 exon 11 or poison exon 11 comprises the sequence set forth in SEQ ID NO: 186. In some embodiments, the target sequence that binds SYNGAP1 exon 11 or poison exon 11 comprises the sequence set forth in SEQ ID NO: 187. In some embodiments, the target sequence that binds SYNGAP1 exon 11 or poison exon 11 comprises the sequence set forth in SEQ ID NO: 188. In some embodiments, the target sequence that binds SYNGAP1 exon 11 or poison exon 11 comprises the sequence set forth in SEQ ID NO: 189. In some embodiments, the target sequence that binds SYNGAP1 exon 11 or poison exon 11 comprises the sequence set forth in SEQ ID NO: 190. In some embodiments, the target sequence that binds SYNGAP1 exon 11 or poison exon 11 comprises the sequence set forth in SEQ ID NO: 191. In some embodiments, the target sequence that binds SYNGAP1 exon 11 or poison exon 11 comprises the sequence set forth in SEQ ID NO: 192. In some embodiments, the target sequence that binds SYNGAP1 exon 11 or poison exon 11 comprises the sequence set forth in SEQ ID NO: 193. In some embodiments, the target sequence that binds SYNGAP1 exon 11 or poison exon 11 comprises the sequence set forth in SEQ ID NO: 194. In some embodiments, the target sequence that binds SYNGAP1 exon 11 or poison exon 11 comprises the sequence set forth in SEQ ID NO: 195. In some embodiments, the target sequence that binds SYNGAP1 exon 11 or poison exon 11 comprises the sequence set forth in SEQ ID NO: 196. In some embodiments, the target sequence that binds SYNGAP1 exon 11 or poison exon 11 comprises the sequence set forth in SEQ ID NO: 197. In some embodiments, the target sequence that binds SYNGAP1 exon 11 or poison exon 11 comprises the sequence set forth in SEQ ID NO: 198. In some embodiments, the target sequence that binds SYNGAP1 exon 11 or poison exon 11 comprises the sequence set forth in SEQ ID NO: 199. In some embodiments, the target sequence that binds SYNGAP1 exon 11 or poison exon 11 comprises the sequence set forth in SEQ ID NO: 200.

In some embodiments, the target sequence that binds SYNGAP1 exon 11 or poison exon 11 comprises the sequence set forth in SEQ ID NO: 201. In some embodiments, the target sequence that binds SYNGAP1 exon 11 or poison exon 11 comprises the sequence set forth in SEQ ID NO: 202. In some embodiments, the target sequence that binds SYNGAP1 exon 11 or poison exon 11 comprises the sequence set forth in SEQ ID NO: 203. In some embodiments, the target sequence that binds SYNGAP1 exon 11 or poison exon 11 comprises the sequence set forth in SEQ ID NO: 204. In some embodiments, the target sequence that binds SYNGAP1 exon 11 or poison exon 11 comprises the sequence set forth in SEQ ID NO: 205. In some embodiments, the target sequence that binds SYNGAP1 exon 11 or poison exon 11 comprises the sequence set forth in SEQ ID NO: 206. In some embodiments, the target sequence that binds SYNGAP1 exon 11 or poison exon 11 comprises the sequence set forth in SEQ ID NO: 207. In some embodiments, the target sequence that binds SYNGAP1 exon 11 or poison exon 11 comprises the sequence set forth in SEQ ID NO: 208. In some embodiments, the target sequence that binds SYNGAP1 exon 11 or poison exon 11 comprises the sequence set forth in SEQ ID NO: 209. In some embodiments, the target sequence that binds SYNGAP1 exon 11 or poison exon 11 comprises the sequence set forth in SEQ ID NO: 210.

In some embodiments, the target sequence that binds SYNGAP1 exon 11 or poison exon 11 comprises the sequence set forth in SEQ ID NO: 211. In some embodiments, the target sequence that binds SYNGAP1 exon 11 or poison exon 11 comprises the sequence set forth in SEQ ID NO: 212. In some embodiments, the target sequence that binds SYNGAP1 exon 11 or poison exon 11 comprises the sequence set forth in SEQ ID NO: 213. In some embodiments, the target sequence that binds SYNGAP1 exon 11 or poison exon 11 comprises the sequence set forth in SEQ ID NO:214. In some embodiments, the target sequence that binds SYNGAP1 exon 11 or poison exon 11 comprises the sequence set forth in SEQ ID NO: 215. In some embodiments, the target sequence that binds SYNGAP1 exon 11 or poison exon 11 comprises the sequence set forth in SEQ ID NO: 216. In some embodiments, the target sequence that binds SYNGAP1 exon 11 or poison exon 11 comprises the sequence set forth in SEQ ID NO: 217. In some embodiments, the target sequence that binds SYNGAP1 exon 11 or poison exon 11 comprises the sequence set forth in SEQ ID NO: 218. In some embodiments, the target sequence that binds SYNGAP1 exon 11 or poison exon 11 comprises the sequence set forth in SEQ ID NO: 219. In some embodiments, the target sequence that binds SYNGAP1 exon 11 or poison exon 11 comprises the sequence set forth in SEQ ID NO: 220.

In some embodiments, the target sequence that binds SYNGAP1 exon 11 or poison exon 11 comprises the sequence set forth in SEQ ID NO: 221. In some embodiments, the target sequence that binds SYNGAP1 exon 11 or poison exon 11 comprises the sequence set forth in SEQ ID NO: 222. In some embodiments, the target sequence that binds SYNGAP1 exon 11 or poison exon 11 comprises the sequence set forth in SEQ ID NO: 223. In some embodiments, the target sequence that binds SYNGAP1 exon 11 or poison exon 11 comprises the sequence set forth in SEQ ID NO: 224. In some embodiments, the target sequence that binds SYNGAP1 exon 11 or poison exon 11 comprises the sequence set forth in SEQ ID NO: 225. In some embodiments, the target sequence that binds SYNGAP1 exon 11 or poison exon 11 comprises the sequence set forth in SEQ ID NO: 226. In some embodiments, the target sequence that binds SYNGAP1 exon 11 or poison exon 11 comprises the sequence set forth in SEQ ID NO: 227. In some embodiments, the target sequence that binds SYNGAP1 exon 11 or poison exon 11 comprises the sequence set forth in SEQ ID NO: 228. In some embodiments, the target sequence that binds SYNGAP1 exon 11 or poison exon 11 comprises the sequence set forth in SEQ ID NO: 229. In some embodiments, the target sequence that binds SYNGAP1 exon 11 or poison exon 11 comprises the sequence set forth in SEQ ID NO: 230.

In some embodiments, the target sequence that binds SYNGAP1 exon 11 or poison exon 11 comprises the sequence set forth in SEQ ID NO: 231. In some embodiments, the target sequence that binds SYNGAP1 exon 11 or poison exon 11 comprises the sequence set forth in SEQ ID NO: 232. In some embodiments, the target sequence that binds SYNGAP1 exon 11 or poison exon 11 comprises the sequence set forth in SEQ ID NO: 233. In some embodiments, the target sequence that binds SYNGAP1 exon 11 or poison exon 11 comprises the sequence set forth in SEQ ID NO: 234. In some embodiments, the target sequence that binds SYNGAP1 exon 11 or poison exon 11 comprises the sequence set forth in SEQ ID NO: 235. In some embodiments, the target sequence that binds SYNGAP1 exon 11 or poison exon 11 comprises the sequence set forth in SEQ ID NO: 236. In some embodiments, the target sequence that binds SYNGAP1 exon 11 or poison exon 11 comprises the sequence set forth in SEQ ID NO: 237. In some embodiments, the target sequence that binds SYNGAP1 exon 11 or poison exon 11 comprises the sequence set forth in SEQ ID NO: 238. In some embodiments, the target sequence that binds SYNGAP1 exon 11 or poison exon 11 comprises the sequence set forth in SEQ ID NO: 239. In some embodiments, the target sequence that binds SYNGAP1 exon 11 or poison exon 11 comprises the sequence set forth in SEQ ID NO: 240.

In some embodiments, the target sequence that binds SYNGAP1 exon 11 or poison exon 11 comprises the sequence set forth in SEQ ID NO: 241. In some embodiments, the target sequence that binds SYNGAP1 exon 11 or poison exon 11 comprises the sequence set forth in SEQ ID NO: 242. In some embodiments, the target sequence that binds SYNGAP1 exon 11 or poison exon 11 comprises the sequence set forth in SEQ ID NO: 243. In some embodiments, the target sequence that binds SYNGAP1 exon 11 or poison exon 11 comprises the sequence set forth in SEQ ID NO: 244. In some embodiments, the target sequence that binds SYNGAP1 exon 11 or poison exon 11 comprises the sequence set forth in SEQ ID NO: 245. In some embodiments, the target sequence that binds SYNGAP1 exon 11 or poison exon 11 comprises the sequence set forth in SEQ ID NO: 246. In some embodiments, the target sequence that binds SYNGAP1 exon 11 or poison exon 11 comprises the sequence set forth in SEQ ID NO: 247. In some embodiments, the target sequence that binds SYNGAP1 exon 11 or poison exon 11 comprises the sequence set forth in SEQ ID NO: 248. In some embodiments, the target sequence that binds SYNGAP1 exon 11 or poison exon 11 comprises the sequence set forth in SEQ ID NO: 249. In some embodiments, the target sequence that binds SYNGAP1 exon 11 or poison exon 11 comprises the sequence set forth in SEQ ID NO: 250.

In some embodiments, the target sequence that binds SYNGAP1 exon 11 or poison exon 11 comprises the sequence set forth in SEQ ID NO: 251. In some embodiments, the target sequence that binds SYNGAP1 exon 11 or poison exon 11 comprises the sequence set forth in SEQ ID NO: 252. In some embodiments, the target sequence that binds SYNGAP1 exon 11 or poison exon 11 comprises the sequence set forth in SEQ ID NO: 253. In some embodiments, the target sequence that binds SYNGAP1 exon 11 or poison exon 11 comprises the sequence set forth in SEQ ID NO: 254. In some embodiments, the target sequence that binds SYNGAP1 exon 11 or poison exon 11 comprises the sequence set forth in SEQ ID NO: 255. In some embodiments, the target sequence that binds SYNGAP1 exon 11 or poison exon 11 comprises the sequence set forth in SEQ ID NO: 256. In some embodiments, the target sequence that binds SYNGAP1 exon 11 or poison exon 11 comprises the sequence set forth in SEQ ID NO: 257. In some embodiments, the target sequence that binds SYNGAP1 exon 11 or poison exon 11 comprises the sequence set forth in SEQ ID NO: 258. In some embodiments, the target sequence that binds SYNGAP1 exon 11 or poison exon 11 comprises the sequence set forth in SEQ ID NO: 259. In some embodiments, the target sequence that binds SYNGAP1 exon 11 or poison exon 11 comprises the sequence set forth in SEQ ID NO: 260.

In some embodiments, the target sequence that binds SYNGAP1 exon 11 or poison exon 11 comprises the sequence set forth in SEQ ID NO: 261. In some embodiments, the target sequence that binds SYNGAP1 exon 11 or poison exon 11 comprises the sequence set forth in SEQ ID NO: 262. In some embodiments, the target sequence that binds SYNGAP1 exon 11 or poison exon 11 comprises the sequence set forth in SEQ ID NO: 263. In some embodiments, the target sequence that binds SYNGAP1 exon 11 or poison exon 11 comprises the sequence set forth in SEQ ID NO: 264. In some embodiments, the target sequence that binds SYNGAP1 exon 11 or poison exon 11 comprises the sequence set forth in SEQ ID NO: 265. In some embodiments, the target sequence that binds SYNGAP1 exon 11 or poison exon 11 comprises the sequence set forth in SEQ ID NO: 266. In some embodiments, the target sequence that binds SYNGAP1 exon 11 or poison exon 11 comprises the sequence set forth in SEQ ID NO: 267. In some embodiments, the target sequence that binds SYNGAP1 exon 11 or poison exon 11 comprises the sequence set forth in SEQ ID NO: 268. In some embodiments, the target sequence that binds SYNGAP1 exon 11 or poison exon 11 comprises the sequence set forth in SEQ ID NO: 269. In some embodiments, the target sequence that binds SYNGAP1 exon 11 or poison exon 11 comprises the sequence set forth in SEQ ID NO: 270.

In some embodiments, the target sequence that binds SYNGAP1 exon 11 or poison exon 11 comprises the sequence set forth in SEQ ID NO: 271. In some embodiments, the target sequence that binds SYNGAP1 exon 11 or poison exon 11 comprises the sequence set forth in SEQ ID NO: 272. In some embodiments, the target sequence that binds SYNGAP1 exon 11 or poison exon 11 comprises the sequence set forth in SEQ ID NO: 273. In some embodiments, the target sequence that binds SYNGAP1 exon 11 or poison exon 11 comprises the sequence set forth in SEQ ID NO: 274. In some embodiments, the target sequence that binds SYNGAP1 exon 11 or poison exon 11 comprises the sequence set forth in SEQ ID NO: 275. In some embodiments, the target sequence that binds SYNGAP1 exon 11 or poison exon 11 comprises the sequence set forth in SEQ ID NO: 276. In some embodiments, the target sequence that binds SYNGAP1 exon 11 or poison exon 11 comprises the sequence set forth in SEQ ID NO: 277. In some embodiments, the target sequence that binds SYNGAP1 exon 11 or poison exon 11 comprises the sequence set forth in SEQ ID NO: 278. In some embodiments, the target sequence that binds SYNGAP1 exon 11 or poison exon 11 comprises the sequence set forth in SEQ ID NO: 279.

### Stem Loops

The engineered snRNA (esnRNA) system disclosed herein can comprise a stem loop (SL) which includes compensatory modifications to a native snRNA stem loop. These modifications result in increased stability of the engineered small nuclear ribonuclear protein complex (esnRNP) compared to snRNP comprising an unmodified stem loop. A SL disclosed herein can be derived from any snRNP such as U1-U12. In one embodiment, the SL is a human or mouse U7 SL. In one embodiment, the SL is a human SL. In one embodiment, the SL is a mouse SL. In some embodiments, the SL is a human and mouse SL. In some embodiments, the SL is a non-human SL, e.g., a mouse SL, a pig, a sheep SL, a goat SL, a cow SL, a dog SL, a cat SL, a horse SL, or a combination thereof. In some embodiments, the SL sequence is not a native stem loop sequence. In some embodiments, the nucleic acid sequence of the SL is at least 65%, 70%, 75%, 80%, 85%, 90%, 95%, 96%, 97%, 98%, 99% or 100% (or any percentage in between) is not a native stem loop sequence. Stem loops are described in WO2023168458, the contents of which are incorporated herein by reference in its entirety for examples of SL sequences that may be used in the constructs described herein.

In some embodiments, a human SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 84)
ggctttctggctccttaccggaaagcc,
(SEQ ID NO: 85)
ggctttctgggaggttaccggaaagcc,
(SEQ ID NO: 86)
ggctttctggcctccttaccggaaagcc,
(SEQ ID NO: 87)
ggctttctggggaggttaccggaaagcc,
(SEQ ID NO: 88)
ggctttctggctggctaccggaaagcc,
(SEQ ID NO: 89)
ggctttctggcttccccggaaagcc,
(SEQ ID NO: 90)
ggctttctggcttcttcccggaaagcc,
(SEQ ID NO: 91)
ggctttctggcaacttaccggaaagcc,
(SEQ ID NO: 92)
ggctttctggttcggtaccggaaagcc,
(SEQ ID NO: 93)
ggctttctggaagccttaccggaaagcc,
(SEQ ID NO: 94)
ggctttctggcttcttaccggaaagcc,
or
(SEQ ID NO: 95)
GGCTTTCTGGCCTCCGCCGGAAAGCCCCT.

In some embodiments, a human SL comprises the sequence set forth in SEQ ID NO: 84. In some embodiments, a human SL comprises the sequence set forth in SEQ ID NO: 85. In some embodiments, a human SL comprises the sequence set forth in SEQ ID NO: 86. In some embodiments, a human SL comprises the sequence set forth in SEQ ID NO: 87. In some embodiments, a human SL comprises the sequence set forth in SEQ ID NO: 88. In some embodiments, a human SL comprises the sequence set forth in SEQ ID NO: 89. In some embodiments, a human SL comprises the sequence set forth in SEQ ID NO: 90. In some embodiments, a human SL comprises the sequence set forth in SEQ ID NO: 91. In some embodiments, a human SL comprises the sequence set forth in SEQ ID NO: 92. In some embodiments, a human SL comprises the sequence set forth in SEQ ID NO: 93. In some embodiments, a human SL comprises the sequence set forth in SEQ ID NO: 94. In some embodiments, a human SL comprises the sequence set forth in SEQ ID NO: 95.

In some embodiments, a murine SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 96)
ggctttctggctccttaccggaaagcccct
(SEQ ID NO: 97)
Ggttttctgacctccgtcggaaaacccct,
(SEQ ID NO: 98)
ggttttctgacctccttcggtcggaaaacccct,
(SEQ ID NO: 99)
Ggttttctgacctccgtcggaaaacc,
(SEQ ID NO: 100)
GGTTTTCTGACACTCCGTCGGAAAACCCCT,
(SEQ ID NO: 101)
GGTTTTCTGATCTCCATCGGAAAACCCCT,
or
(SEQ ID NO: 102)
GGTTTTCCGACCTCCGTCGGAAAACCCCT.

In some embodiments, a murine SL comprises the sequence set forth in SEQ ID NO: 96. In some embodiments, a murine SL comprises the sequence set forth in SEQ ID NO: 97. In some embodiments, a murine SL comprises the sequence set forth in SEQ ID NO: 98. In some embodiments, a murine SL comprises the sequence set forth in SEQ ID NO: 98. In some embodiments, a murine SL comprises the sequence set forth in SEQ ID NO: 100. In some embodiments, a murine SL comprises the sequence set forth in SEQ ID NO: 101. In some embodiments, a murine SL comprises the sequence set forth in SEQ ID NO: 102.

In some embodiments, a human or murine SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 103)
GGCTTTCTGGCACTCCACCGGAAAGCCCCT,
(SEQ ID NO: 104)
GGCTTTCTGGCACTCCGCCGGAAAGCCCCT,
or
(SEQ ID NO: 105)
GGCTTTCTGGCCTCCACCGGAAAGCCCCT.

In some embodiments, a human or murine SL comprises the sequence set forth in SEQ IID NO: 103. In some embodiments, a human or murine SL comprises the sequence set forth in SEQ IID NO: 104. In some embodiments, a human or murine SL comprises the sequence set forth in SEQ IID NO: 105.

In some embodiments, a dog or cat SL comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleotide sequence

(SEQ ID NO: 106)
GGTTTTCCGGTCTCCACCGGAAAGCCCCC.

In some embodiments, a cow, sheep, or goat SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 107)
GGCTTTCCGGTCTCCACCGGAAAGCCCCT,
or
(SEQ ID NO: 108)
GGCTTTCCGGCCTCCGCCGGAAAGCCCCT.

In some embodiments, a cow, sheep, or goat SL comprises the sequence set forth in SEQ ID NO: 107. In some embodiments, a cow, sheep, or goat SL comprises the sequence set forth in SEQ ID NO: 108.

In some embodiments, a pig SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 109)
GGTTTTCCGGTCTCCACCGGAAAACCCTT.
(SEQ ID NO: 110)
GGTTTTCCGTGCTCCCACGGAAAACCCTT,
(SEQ ID NO: 111)
GGTTTTCCGGCCTCCGCCGGAAAACCCTT,
(SEQ ID NO: 112)
GGTTTTCCGTGACTCCCACGGAAAACCCTT,
or
(SEQ ID NO: 113)
GGTTTTCCGGCACTCCGCCGGAAAACCCTT.

In some embodiments, a pig SL comprises the sequence set forth in SEQ ID NO: 109. In some embodiments, a pig SL comprises the sequence set forth in SEQ ID NO: 110. In some embodiments, a pig SL comprises the sequence set forth in SEQ ID NO: 111. In some embodiments, a pig SL comprises the sequence set forth in SEQ ID NO: 112. In some embodiments, a pig SL comprises the sequence set forth in SEQ ID NO: 113.

In some embodiments, a horse SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 114)
GGTCTTCCGGTCTCCTCCGGAAGGCCCCC,
or
(SEQ ID NO: 115)
GGTCTTCCGGCTCCCCGGAAGGCCCCC.

In some embodiments, a horse SL comprises the sequence set forth in SEQ ID NO: 114. In some embodiments, a horse SL comprises the sequence set forth in SEQ ID NO: 115.

In some embodiments, a sheep SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 116)
GGCTTTCCGTGCTCCCACGGAAAGCCCCT,
(SEQ ID NO: 117)
GGCTTTCCGTGACTCCCACGGAAAGCCCCT,
or
(SEQ ID NO: 118)
GGCTTTCCGGCACTCCGCCGGAAAGCCCCT.

In some embodiments, a sheep SL comprises the sequence set forth in SEQ ID NO: 116. In some embodiments, a sheep SL comprises the sequence set forth in SEQ ID NO: 117. In some embodiments, a sheep SL comprises the sequence set forth in SEQ ID NO: 118.

In some embodiments, stem loops provide for enhanced stability of an snRNA relative to an snRNA comprising a native stem loop. In some embodiments is a native snRNA stem loop comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 119)
Ggttttctgacttcggtcggaaaacccct,
(SEQ ID NO: 120)
ggttttctgacttcggtcggaaaacc,
(SEQ ID NO: 121)
Ggctttctggctttttaccggaaagcc,
(SEQ ID NO: 122)
ggctttctggctttttaccggaaagccCCT,
(SEQ ID NO: 123)
GGCTTTCCGGCCTCCGCCGGAAAGCCCCT,
or
(SEQ ID NO: 124)
GGCTTTCCGGCCTCCGCCGGAAAGCC.

In some embodiments is a native snRNA stem loop comprises the sequence set forth in SEQ ID NO: 119. In some embodiments is a native snRNA stem loop comprises the sequence set forth in SEQ ID NO: 120. In some embodiments is a native snRNA stem loop comprises the sequence set forth in SEQ ID NO: 121. In some embodiments is a native snRNA stem loop comprises the sequence set forth in SEQ ID NO: 122.

### 5′ Interaction Stability Domain

The SL disclosed herein possesses more effective folding and annealing properties with a 5′ interaction stability domain (5′ISD) and this in turn results in increased stability of the esnRNA compared to a non-engineered snRNA. The 5′ ISD has nucleotides that are complementary to the nucleotides within the SL, and without wishing to be bound by theory, an interaction between the 5′ISD and SL is predicted to form secondary structure that protects the 5′ end of an snRNA. In some aspects the 5′ ISD anneals and/or hybridizes to an SL of the disclosure. In some aspects the 5′ISD is a sequence having complementarity and/or reverse complementarity to a sequence present in an SL of the disclosure. In some aspects a 5′ISD disclosed herein can comprise or consist of one of the following nucleotide sequences:

ggagt,
cctct,
ggaggt,
cctcct,
agccag,
ggaag,
gaagaag,
gttg,
ccgaa,
taaggag,
gaag,
and
ggctt.

### Sm Binding Domains

The snRNA systems disclosed herein utilize an Sm binding domain (SmBD). The Sm protein ring that assembles around the Sm binding domain (SmBD) to form an snRNP includes SmB/B′, SmD1, SmD2, SmD3, SmE, SmF, and SmG. The U7 Sm binding site recruits endogenous RNA binding factors and can be replaced with a non-U7 snRNA to make the esnRNA more stable. In one embodiment, the SmBD a U1 SmBD, a U2 SmBD, a U4 SmBD, or a SmBD. In another embodiment, the SmBD is derived from a pseudo snRNA. In another embodiment, the SmBD is a nucleotide sequence comprising ATTTTT. In another embodiment, the SmBD comprises the nucleotide sequence AATTTTTGG, AATTTGTGG, AATTTGTGG, AATTTCTGG, GATTTTTGG, AATTTTTGA, AATTTTTTG, AATTTTTGGAGCA (SEQ ID NO: 160), or AATTTTTGGAGTA (SEQ ID NO: 161).

### Promoter Sequences

Gene therapy and RNA-targeting snRNA gene therapy compositions of the disclosure comprise promoter sequences derived from an snRNA. A “promoter” is a regulatory sequence that is a region of a polynucleotide sequence at which initiation and rate of transcription are controlled. It may contain genetic elements at which regulatory proteins and molecules may bind such as RNA polymerase and other transcription factors.

The snRNA systems disclosed herein may comprise an snRNA promoter from any of U1-U12. In one embodiment, the snRNA promoter is a U7 promoter. In another embodiment, the U7 promoter is a human U7 promoter (hU7) or a mouse U7 promoter (mU7). In another embodiment, the U7 promoter is an endogenous human U7 promoter at least 65%, 70%, 75%, 80%, 85%, 90%, 95%, 96%, 97%, 98%, 99% or 100% (or any percentage in between) identical to SEQ ID NO: 135:

TACTGCCGAATCCAGGTCTCCGGGCTTAACAACAACGAAGGGGCTGTGA
CTGGCTGCTTTCTCAACCAATCAGCACCGAACTCATTTGCATGGGCTGA
GAACAAATGTTCGCGAACTCTAGAAATGAATGACTTAAGTAAGTTCCTT
AGAATATTATTTTTCCTACTGAAAGTTACCACATGCGTCGTTGTTTATA
CAGTAATAGGAACAAGAAAAAAGTCACCTAAGCTCACCCTCATCAATTG
TGGAGTTCCTTTATATCCCATCTTCTCTCCAAACACATACGCA.

In one embodiment, the snRNA promoter is a U1 promoter. In another embodiment, the U1 promoter is a human U1 promoter or a mouse U1 promoter.

In another embodiment, the same snRNA promoter drives expression of each copy of an snRNA insert. In another embodiment, each copy of an snRNA insert is the same. In another embodiment, different snRNA promoters drive each copy of an snRNA insert. In one embodiment, a 2×snRNA comprises a mouse U7 promoter driving one copy of an snRNA insert and a mouse U1 promoter drives the other copy of an snRNA insert.

In other aspects, the snRNA promoter is a PolII promoter or a PolIII promoter.

In other aspects, the snRNA promoter comprises, consists essentially of, or consists of a nucleic acid sequence at least 65%, 70%, 75%, 80%, 85%, 90%, 95%, 96%, 97%, 98%, 99% or 100% (or any percentage in between) identical to a promoter and/or promoter sequence listed in Table 4, which follows:

TABLE 4
Illustrative Promoter Sequences

| | | SEQ |
|---|---|---|
| Name | Sequence | ID NO |
| hU1 | AAGGACCAGCTTCTTTGGGAGAGAACAGACGCAGGGGGGGGAGG | 125 |
| | GAAAAAGGGAGAGGCAGACGTCACTTCCCCTTGGCGGCTCTGGCA | |
| | GCAGATTGGTCGGTTGAGTGGCAGAAAGGCAGACGGGGACTGGG | |
| | CAAGGCACTGTCGGTGACATCACGGACAGGGCGACTTCTATGTAG | |
| | ATGAGGCAGCGCAGAGGCTGACGTCTTCGCCACTTGCTGCTTCAC | |
| | CACGAAGGAGTTCCCGTGCCCTGGGAGCGGGTTCAGGACCGCTGA | |
| | TCGGAAGTGAGAATCCCAGCTGTGTGTCAGGGCTGGAAAGGGCTC | |
| | GGGAGTGCGCGGGGCAAGTGACCGTGTGTGTAAAGAGTGAGGCG | |
| | TATGAGGCTGTGTCGGGGCAGAGGCacaacgtttc | |
| hU2 | CCACGCCCTCTGTGAAAGGGCGGGGCATGCAAATTCGAAATGAAA | \|126 |
| | GCCCGGGAACGCCGGAAGAAGCACGGGTGTAAGATTTCCCTTTTC | |
| | AAAGGCAGAGAATAAGAAATCAGCCCGAGAGTGTAAGGGCGTCA | |
| | ATAGCGCTGTGGACGAGACAGAGGGAATGGGGCAAGGAGCGAGG | |
| | CTGGGGCTCTCACCGCGACTTGAATGTGGATGAGAGTGGGACGGT | |
| | GACGGCGGGCGCGAAGGCGAGCGC | |
| hU4 | TTCGCAGTCTCTGAATTAAGTCTATTAGCATGTTCCTCCCATAGTG | 127 |
| | CTTTGCTTCATATCAACAAAAACCTAGCTAAGTGAAATCAGCAAC | |
| | GATATGCAGAAACCACCTACGCAGGTCACAAACATCTTTCTATGA | |
| | TTGTATAATTTTCAAGCAAGCAATAAGTGAAGATTTTTCCATAGGC | |
| | CCTAAACTCACCTTTGCGAAATAGGAAGCTGGTTTATTGGGAGTG | |
| | ATGAGCAGGGGGCGTAACAAATT | |
| hU5 | GCAGCAAGGCCTCCACTTCACCCCCTAAAGGTTGCCCCAAGAGCA | \|128 |
| | CCGTGTGACTGCTAAGGTATTTCCGGAGTCTAAAGACGATTATTCA | |
| | GGTCTCATTTGCATACCCATAATACACTGCAAACAGTATTTTTTTC | |
| | GGAAAAACATTTATATATTGCTTGACATTTTTAAGTATGAGAATTT | |
| | TGCATGCAGAATTTTTTTGTATAAACTTTCTCAGGTAGTAACCCTT | |
| | GGGATTAGTAGACACCATCAGTGTACTAGGAATTGCAGTTACCCG | |
| | AAAATTGAGTTACAGAAGTAACTGGT | |
| hU6 | Gtttattacagggacagcagagatccagtttggttaattaaggtaccgagggcctatttcccatgattccttcatat | 129 |
| | ttgcatatacgatacaaggctgttagagagataattagaattaatttgactgtaaacacaaagatattagtacaaaa | |
| | tacgtgacgtagaaagtaataatttcttgggtagtttgcagttttaaaattatgttttaaaatggactatcatatgctta | |
| | ccgtaacttgaaagtatttcgatttcttggctttatatatcttgtggaaaggacgaaacacc | |
| h7sk | CTGCAGTATTTAGCATGCCCCACCCATCTGCAAGGCATTCTGGATA | 130 |
| | GTGTCAAAACAGCCGGAAATCAAGTCCGTTTATCTCAAACTTTAG | |
| | CATTTTGGGAATAAATGATATTTGCTATGCTGGTTAAATTAGATTT | |
| | TAGTTAAATTTCCTGCTGAAGCTCTAGTACGATAAGTAACTTGACC | |
| | TAAGTGTAAAGTTGAGATTTCCTTCAGGTTTATATAGCTTGTGCGC | |
| | CGCCTGGGTACCTC | |
| tRNA(Val) | CAGGACTAGTCTTTTAGGTCAAAAAGAAGAAGCTTTGTAACCGTT | 131 |
| | GGTTTCCGTAGTGTAGTGGTTATCACGTTCGCCTAACACGCGAAAG | |
| | GTCCCCGGTTCGAAG | |
| mU1 | TTGTTCCTCTTAGTGTTAATTCACACTAAAGACTGTGCATCCGACT | 132 |
| | CCTACATTTATGAAAGTAAATGCCTGTTGTTAGAACAAAAAAGGC | |
| | TACAGAACAAAAAACAAAGCGAAATACCATCTGCTTTAGGTTCAG | |
| | TGTGGTATTTTCCCGCTGACAGGGAGGCGGGTTTTTGGGTACAGG | |
| | AAACGAGTCACTATGGAGGCGGTACTATGTAGATGAGAATTCAGG | |
| | TGCAAACTGGGAAAAGCAACTGCTTCCAAATATTTGTGATTTTTAC | |
| | AGTGTAGTTTTGGAAAAACTCTTAGCCTACCAATTCTTCTAAGTGT | |
| | TTTAAAATGTGGGAGCCAGTACACATGAAGTTATAGAGTGTTTTA | |
| | ATGAGGCTTAAATATTTACCGTAACTATGAAATGCTACGCATATCA | |
| | TGCTGTTCAGGCTCCGTGGCCACGCAACTC | |
| mU7+ | TaacaacataggagctgtgattggctgttttcagccaatcagcactgActcatttgcatagcctttacaagcggt | 133 |
| extra | cacaaactcaagaaacgagcggttttaatagtcttttagaatattgtttatcgaaccgaataaggaactgtgctttg | |
| 3′ | tgattcacatatcagtggaggggtgtggaaatggcaccttgatctcaccctcatcgaaagtggagttgatgtcct | |
| | Tccctggctcgctacagacgcacttccgcaa | |
| mU7 | taacaacataggagctgtgattggctgttttcagccaatcagcactgActcatttgcatagcctttacaagcggtc | 134 |
| | acaaactcaagaaacgagcggttttaatagtcttttagaatattgtttatcgaaccgaataaggaactgtgctttgt | |
| | gattcacatatcagtggaggggtgtggaaatggcaccttgatctcaccctcatcgaaagtggagttgatgtcct | |
| | Tccctggctcgctacagacgcacttccgc | |
| hU7 | TACTGCCGAATCCAGGTCTCCGGGCTTAACAACAACGAAGGGGCT | 135 |
| | GTGACTGGCTGCTTTCTCAACCAATCAGCACCGAACTCATTTGCAT | |
| | GGGCTGAGAACAAATGTTCGCGAACTCTAGAAATGAATGACTTAA | |
| | GTAAGTTCCTTAGAATATTATTTTTCCTACTGAAAGTTACCACATG | |
| | CGTCGTTGTTTATACAGTAATAGGAACAAGAAAAAAGTCACCTAA | |
| | GCTCACCCTCATCAATTGTGGAGTTCCTTTATATCCCATCTTCTCTC | |
| | CAAACACATACGCA | |
| mU5 | TGCCAACAGCTTTGAAATCCTCTGTGCTCTTGTGCCAATCCCACCC | 136 |
| | TCAGAACAGGGAGTCTGTCGGTTATGTTTTCGTCCTTTACAGTTCT | |
| | TCGCCGTCTAATCCATTGGAAACCTCCTTTATTTAAAGGAAACCGT | |
| | GTCCAAATTCAGTGTACAGAGGTGCAAACCTCCGCCCCGCTCCTTG | |
| | TAGGAAGCCCACGGGAACTGGGCCAATCGGACGGCGCTTACTCGG | |
| | CTCATTTACATACCCATAACACACCGCGGCTAATGCAAATATTTTC | |
| | GTGAAAAGATCTTCCATTACTCGAAGTTTTAAAACTAAAAACAAA | |
| | ATATTATTTCAAAAGATCATGAAGAAATTTCTGTCGGTAACAGCA | |
| | GTTTCAATTGATAAATCACCATCACTTATCTAGGAGGTTCTGTTAC | |
| | TCTAGAAGTGAATTAAGCAGGACAGCTGT | |
| mU2 | Gggcggggcatgcaaataactgctctgtggaactctgggagcaaaaacaaaaaactgcaaccaaaacttctc | 137 |
| | ggcctccttgaatcttacaggcttttcgtggcgtaaaggtggtgtactcaatgaagaggagagtctgtgttggct | |
| | gcatgtttgagtcggttggttggtgactgtgaatTAAAGGTGTGGtcggtgttgagtgtatggggcgt | |
| | gtgggcgtagttcggt | |
| mU6 | cgacgccgccatctctaggcccgcgccggccccctcgcacagacttgtgggagaagctcggctactcccctg | 138 |
| | ccccggttaatttgcatataatatttcctagtaactatagaggcttaatgtgcgataaaagacagataatctgttcttt | |
| | ttaatactagctacattttacatgataggcttggatttctataagagatacaaatactaaattattattttaaaaaacag | |
| | cacaaaaggaaactcaccctaactgtaaagtaattgtgtgttttgagactataaatatcccttggagaaaagcctt | |
| | gttt | |
| mH1 | CATgCAAATTACGCGCTgTGCTTTGTGGGAAATCACCCTAAACGTA | 139 |
| | AAATTTATTCCTCTTTcGAGCCTTATAGTGGcGGCCGGTCTACACCC | |
| | TAAA | |
| rU1 | TTACTTCATACTAAAGGCTGTGCATCCGACTCCTAAGTTGATGAAG | 281 |
| | GAAAATGCCTAGTGTTCTTGGAGGCTACAAAACAAAAGACAAAGC | |
| | TAACTACCATCTGCTTATGGGTTCATTGGTATTTTCCAGCTGGCAG | |
| | GGAGGCGGGTTTCCGAGTACAGGAAATGAGTCTCTATGGAGGCGG | |
| | TGCTATGTAGATGAGAATTCAGGAGCAAACTGGGAAAAGCAGCTG | |
| | CTTCCAAATATTTGTGATTTTTGGAGTGTAGTTTTGGGGAAACTCG | |
| | CAGCCTACCAATTCTCCTAAGTGCTTTAGAATATGGAGAGACACT | |
| | GTACATAAAGATATAGAGCTTTTTAATGGAGGCTTAAATTTATACC | |
| | GTATCTACAAAATGCTACATTCACAATGCAGTTCAGGCTCTGTGGC | |
| | ATTGCAACTC | |
| bU7 | GCGCAGGAGCCGCCGAGCTCTTGCTGCGAAGCCTTGTCTGCGTTCT | 282 |
| | TAAAAACTAAAGGGGGCGTGACTGGCTTCCTTATCAGCCAATCGG | |
| | CATCGGGTCATTTGCATAGGGCCCCATCCACCGTTCGTAAACTCTA | |
| | GCCGCGAAGGATTTAAGAGAGTTCCTTAGAACACGGTCTTCCCCT | |
| | CCGAAAGTCGCAGTCTGCCTCGTTATCTGTAGAGCAATAGGAGCT | |
| | AGGAACCGGCCGTCGGGGCTCACCCTCACTGACAGCGGGGTTGGT | |
| | GGCTATGCCGTGCGATCTCCGGGTGCTACGTCTG | |
| sU1 | CGCCGTGCCCTGCTCTGCCCTCCGCACGCTGCTCAGACTCCACACC | 283 |
| | CGTAACGAAGCTCCACCGAATTTTTCGTCTGTCTGTGAAGACGAA | |
| | GAATTGAAAAATCTCAGCTACACTTGGTCTTTGGGGTCGGCGCCA | |
| | GGCAGAGCTAGAACGAACTAGACGCTTGTCCGAAAGCAGTGACGT | |
| | CAAATGACAAGGGACAAGAGGTGGGGATATGTAGATGAGGGGAG | |
| | CGGTGGCTGCTGGCTGGATTTCGTCTTTCGCCGGGTAGAATTTCCC | |
| | CTGAACGTCCAAAGGATGTATCTTGGTCACTGTGAAATAAATAAT | |
| | CCTTCAGGTTCCCATCGAAGGCCGAAATAGCTTGGAATATAATTG | |
| | ACCATAACTCTGTTGTGTGCTGAGTTCGAGGGTGACGTCCCAGACT | |
| | AAGCTTACAACTC | |
| eqU1 | TGCCGCGCAGAATGCAGACTCATCCTGACAAGTCCACGCCAGAAC | 284 |
| | CTTTGCGTCTGCCCGGAAAGGCAAGGAAGTTTAAAGCCTTTCCAG | |
| | ATACCCCTATGGATGATTGTCTTTTAGATCGTCCTGAGGGCTAGCT | |
| | AGAGCGAATTCACCGCTTCTCCGAGAAGCAGAATGACGTCGAGTG | |
| | ACGACAGTGATGACAAGAGGCGGGGATATGTAGATGAAAGACAC | |
| | GGTGACTGCCGGTTGGAGTGAGTTCTTCATGGGGAACACTTTCCAC | |
| | TGAATATCCAAAAGGATGTACCCTGATCTCTGTCATATAAATAACC | |
| | CTTCAATTTCGAACTTAAGGCCTAAAGAACTCGGAATATAATTGA | |
| | CCATGACTTTATAGTGTGTTGAAGACGATGGTGAGGTGCCAGGCG | |
| | AGGGGAGCAACTC | |

In some embodiments, the snRNA promoter comprises the sequence set forth in SEQ ID NO: 125. In some embodiments, the snRNA promoter comprises the sequence set forth in SEQ ID NO: 126. In some embodiments, the snRNA promoter comprises the sequence set forth in SEQ ID NO: 127. In some embodiments, the snRNA promoter comprises the sequence set forth in SEQ ID NO: 128. In some embodiments, the snRNA promoter comprises the sequence set forth in SEQ ID NO: 129. In some embodiments, the snRNA promoter comprises the sequence set forth in SEQ ID NO: 130. In some embodiments, the snRNA promoter comprises the sequence set forth in SEQ ID NO: 131. In some embodiments, the snRNA promoter comprises the sequence set forth in SEQ ID NO: 132. In some embodiments, the snRNA promoter comprises the sequence set forth in SEQ ID NO: 133. In some embodiments, the snRNA promoter comprises the sequence set forth in SEQ ID NO: 134. In some embodiments, the snRNA promoter comprises the sequence set forth in SEQ ID NO: 135. In some embodiments, the snRNA promoter comprises the sequence set forth in SEQ ID NO: 136. In some embodiments, the snRNA promoter comprises the sequence set forth in SEQ ID NO: 137. In some embodiments, the snRNA promoter comprises the sequence set forth in SEQ ID NO: 138. In some embodiments, the snRNA promoter comprises the sequence set forth in SEQ ID NO: 139. In some embodiments, the snRNA promoter comprises the sequence set forth in SEQ ID NO: 281. In some embodiments, the snRNA promoter comprises the sequence set forth in SEQ ID NO: 282. In some embodiments, the snRNA promoter comprises the sequence set forth in SEQ ID NO: 283. In some embodiments, the snRNA promoter comprises the sequence set forth in SEQ ID NO: 284.

### Terminator Sequences

The snRNA systems disclosed herein may comprise an snRNA downstream terminator (DT). Downstream terminators define the end of a transcriptional unit, such as an esnRNA or snRNA. In another embodiment the snRNA DT is a U7 DT comprising the sequence

(SEQ ID NO: 147)
CCTCTTATGATGTTTGTTGCCAATGATAGATTGTTTTCACTGTGCAAAA
ATTATGGGTAGTTTTGGTGGTCTTGATGCAGTTGTAAGCTTGGAG.

In one embodiment, the snRNA comprises the SL or eSL, one or more promoters, the TS targeting a SCN1A or SYNGAP1 RNA molecule, the SmBD, the 5′ISD, and the DT. In another embodiment, the snRNA comprises a native stem loop, one or more promoters, the TS targeting SCN1A or SYNGAP1, the SmBD, and the DT. The promoter and DT sequences provided herein may be mixed and matched in any combination.

In some embodiments, the DT comprises, consists essentially of, or consists of a nucleic acid sequence at least 65%, 70%, 75%, 80%, 85%, 90%, 95%, 96%, 97%, 98%, 99% or 100% (or any percentage in between) identical to a DT sequence listed in Table 5, which follows:

TABLE 5
Illustrative DT Sequences

| | | SEQ |
|---|---|---|
| Name | Sequence | ID NO |
| hU1 | ACTTTCTGGAGTTTCAAAAACAGACTGTACGCCAAGGGTCATATCTTTTT | 140 |
| hU2 | CCGGGGATACAACGTGTTTCCTAAAAGTAGAGGGAGGTAAGAGACGGTAG | 141 |
| hU4 | CTGAATTTTCTTGCAGTTGAACAACAGAGGCTTTTTTTGTGTGTGTGGGG | 142 |
| hU5 | ATATGTGGTAATCCAACAATAGAAATTATTTTTAAGTTTGTGTGTTCCTT | 143 |
| hU6 | TTTTTT | N/A |
| h7sk | TTTTTT | N/A |
| tRNA(Val) | TTTTTT | N/A |
| mU1 | GTTTACTTGGTTTTAAAAATAGCTTGCACTAGCGATACGG | 144 |
| | AATATGGTTATTAGGTTTGTTAGGCATCATGTCGTGTCTT | |
| | ACTATAGAAAAATAACGTAGTGTTCATTTTAGCCTGCCTG | |
| | TATGTGTTAATTTGTCCTTATTGCGCATTGTTCTTGTTAAG | |
| | TCTTCTGTAAGGAGTTGCGGGTTTCAAACTGTCAGTCTGA | |
| | GAGCA | |
| mU7+ | Cccaatttcactggtctacaatgaaagcaaaacagttctcttccccgctccccggtgtgtgagag | 145 |
| | gggctttgatccttctctggtttcctaggaaacgcgtatgtgtac | |
| mU7 | Cccaatttcactggtctacaatgaaagcaaaacagttctcttccccgctccccggtgtgtgagag | 146 |
| | gggctttgatccttctctggtttcctaggaaacgcgtatgtg | |
| hU7 | CCTCTTATGATGTTTGTTGCCAATGATAGATTGTTTTCACT | 147 |
| | GTGCAAAAATTATGGGTAGTTTTGGTGGTCTTGATGCAGT | |
| | TGTAAGCTTGGAG | |
| hU7 | CTTATGATGTTTGTTGCCAATGATAGATTGTTTTCACTGTG | 148 |
| | CAAAAATTATGGGTAGTTTTGGTGGTCTTGATGCAGTTGT | |
| | AAGCTTGGAG | |
| mU5 | CCACATTTTGTGTTTAAAAATAGAAATATTTAAGTGAGAT | 149 |
| | CAGTTTAAATCTGCTTTATCTAGGGTGTCTAACTGCTTGC | |
| | ATCTTTTTAACTTTTCCTTATTTGTGAGAAGGTCTGTAAAA | |
| | CTTATTATATGTTAGTACACTGTAGCTGTCTTCAGACACT | |
| | CCAGAAGAGGGATTCAGATCACCTTACAGATGGTTTTGA | |
| mU2 | CCCTCTGGGGAgtaaagttggttttaaagtcagagcatggtgattgtagggcagtcca | 150 |
| | acttttttaaatatgctgtg | |
| mU6 | TTTTTT | N/A |
| mH1 | TTTTTT | N/A |
| rU1 | ATTTGTTTGGTACTAAAGATAGTTATCAGCCGAACCAGAA | 285 |
| | GGCTAAAATGGCTTCTGATACTTACTTGGCCAATGCCTTT | |
| | TCCCTTTATACTGCTATTGCTTTGTATTCTGAAAAGCATCT | |
| | TAGTGGTTTTTAACTTTCTCTACGTTCCCTCTGCACGTTGA | |
| | GTCTTGAGTTATGTTAAATGGTACGGTACCTAGGATTG | |
| bU7 | CCCACACAAGTGTTTATGAACAATAGAATAATATGTGGG | 286 |
| | TGGGAAGCTTGGTTTTTTTTGATACGGGTTTTTTTTTTTTT | |
| | TTTGGTCTACGTGTTTAGTCCGTTCTTGACGGAGAAGACC | |
| | CGGGAGGTTGGGATTCTTGTCTGGAAATTTCCGGCTCTGT | |
| | CTGATGTAGTTTCCCGTTCGCCTCCCTTCTTTGCTGCGAG | |
| SU1 | GCGTGGTTTGTGCAAGGAAAGACATAGATGCTAAGTTTG | 287 |
| | GAGCTCTGCTAATTTCCTGTTTTTGTAGTCGTTTTCGGGTT | |
| | TATGAGGCAGAGGCAGTAAGCAGTTTTCCTGCAACATGG | |
| | ATAAGCTAGTTTGGACTTTTGGTAAAGGATTTTTTTGTAA | |
| | TGTGGGAGACTTGCTTTTGATCCCTGGTTTGGGAAATTCTC | |
| eqU1 | ACAAACTTGGTCTTAAAAGAAGACTACTTGTTTATGTTCT | 288 |
| | TCTTTCTCTAACTTTTATTTGGGGTTTAGTGGCTTTCTAAC | |
| | ATGGTCATTATGAGGTAAGACCTTGGGGTTGGCCTATTAA | |
| | TTAACACCTTGGGGCTGGCCGTATGGCTGAGTGGTTAAGT | |
| | TTGTGTGCTCTGCTTCGGGTTTGGCCTGTTCTAATTCTG | |

In some embodiments, the DT comprises the sequence set forth in SEQ ID NO: 140. In some embodiments, the DT comprises the sequence set forth in SEQ ID NO: 141. In some embodiments, the DT comprises the sequence set forth in SEQ ID NO: 142. In some embodiments, the DT comprises the sequence set forth in SEQ ID NO: 143. In some embodiments, the DT comprises the sequence set forth in SEQ ID NO: 144. In some embodiments, the DT comprises the sequence set forth in SEQ ID NO: 145. In some embodiments, the DT comprises the sequence set forth in SEQ ID NO: 146. In some embodiments, the DT comprises the sequence set forth in SEQ ID NO: 147. In some embodiments, the DT comprises the sequence set forth in SEQ ID NO: 148. In some embodiments, the DT comprises the sequence set forth in SEQ ID NO: 149. In some embodiments, the DT comprises the sequence set forth in SEQ ID NO: 150. In some embodiments, the DT comprises the sequence set forth in SEQ ID NO: 285. In some embodiments, the DT comprises the sequence set forth in SEQ ID NO: 286. In some embodiments, the DT comprises the sequence set forth in SEQ ID NO: 287. In some embodiments, the DT comprises the sequence set forth in SEQ ID NO: 288. In some embodiments, the DT comprises the sequence set forth in SEQ ID NO: TTTTTT.

In one embodiment, the snRNA is delivered in an AAV vector.

In some embodiments, the AAV vector comprises multiple copies of the snRNA. In some embodiments, the multiple copies of the snRNA are 2, 3, 4, 5, 6, 7, 8, 9, or 10 copies (2×, 3×, 4×, 5×, 6×, 7×, 8×, 9× or 10×) of the snRNA. In some embodiments, the multiple copies of the snRNA are 4 or more copies of the snRNA.

In some embodiments, each snRNA of the multiple copies of snRNA is separated by a nucleic acid buffer sequence derived from human non-coding genomic sequences downstream of an snRNA. In one embodiment, the buffer sequence is derived from human genomic sequences downstream of U7.

In one embodiment, the buffer sequence is one of the following nucleic acid sequences:

buffer 1 (30 bp):
(SEQ ID NO: 151)
CAAACTACAGAGCCAAGTGCTATCCACAGA,
buffer 2 (30 bp):
(SEQ ID NO: 152)
GAGCTTTCTGGGTTGCCATCTCAAGCAGAC,
buffer 3 (30 bp):
(SEQ ID NO: 153)
TACAAGGCCATCAGCTCATACTCACAATTG,
or
a combination thereof.

In another embodiment, the buffer sequence is one of the following nucleic acid sequences:

buffer 1 (100 bp):
(SEQ ID NO: 154)
CAAACTACAGAGCCAAGTGCTATCCACAGAGAGCTTTCTGGGTTGCCA
TCTCAAGCAGACTACAAGGCCATCAGCTCATACTCACAATTGACTTTG
AGAG,
buffer 2 (100 bp):
(SEQ ID NO: 155)
TTGACCACATACGTGCTCTTTCAAAGTTCTGTGTTTGAAGTTATGTTA
GTAACAACTGATGCCCATCCTGCAATGACAAATCCAATTCTCAGTGCA
GCTC,
or
a combination thereof.

In another embodiment, the buffer sequence is one of the following nucleic acid sequences:

buffer 1 (500 bp):
(SEQ ID NO: 156)
CAAACTACAGAGCCAAGTGCTATCCACAGAGAGCTTTCTGGGTTGCCA
TCTCAAGCAGACTACAAGGCCATCAGCTCATACTCACAATTGACTTTG
AGAGTCATTTTCCAATGCTCCTACACACCCCTTCTTCACAATCCCCAA
CAAATCTGAGGCTGGAACTTGGTACCATAACAATCATTACATTATTTC
ACCAGAAGTACACCTTGCCTGGAAGATTGGCATTATAGCATCTTCTAA
CATTGTGAAAGTTAGTGACCAATGAGGAGATCCAAGTCAGTTCCAGTT
GGATTTCTCTATACTCTATAATAAATATATATGGTGTCTTCAACAATA
GGACTTTGCCATCCAGTGATGCTAAAAATCAATAACAATGGCAATAAC
CTGCCCTGTTTGGAAAGCCTCTGGCTTCCATGACTAACAATTCAAGGC
AGGTCTCCTATACCTAGTACTGAGATTTTTATTTGATAAACTATATCT
TCTGGGAGGAGAAGCATTGT,
buffer 2 (500 bp):
(SEQ ID NO: 157)
TTGACCACATACGTGCTCTTTCAAAGTTCTGTGTTTGAAGTTATGTTA
GTAACAACTGATGCCCATCCTGCAATGACAAATCCAATTCTCAGTGCA
GCTCTCTGAAATAGTTTTGCTTTCTCTCTCTAGGTCTGTTCTATACTC
CTAACTCTCCAGGAGTTTACAAGGAATAAAATCTCTTCCAAATGCTTT
CTGTTGCAACAACTGGACCATACTGAAAGCTGAGGCCCACAATTGCAA
TCTAGGTTAGCAGGTAATCATTGTTGGTGAGGTCCTCCCTTTCCCCAG
GCTCGTGTTTGTATTGGGGAGCAGGAAATTTTTGCTAGAGCAGCACTG
CCATCTCTCTACACTCCACCTGATTGGTGGGATGGACCAGAGAAATGG
ACATTCCCAACACAGTCCCTCCTTTCACATCTGCTCACCTGCCCACAG
GATACTTTCCACCATGCATACTGGGCTCTGCACCAACCATTCAGCAGT
GATGAAGAGGAAACTTGAAC,
or
a combination thereof.

The 100 bp and 500 bp buffer 1 sequences are derived from a sequence starting 100 bp downstream of the Mus musculus U7 pseudogene 8 (Location Chromosome 14: 4,409,359-4,409,421 reverse strand. GRCm39:CM001007.3). The 100 bp and 500 bp buffer 2 sequences are derived from the sequence starting 130 bp downstream of human U7 pseudogene 5 (Chromosome X: 140,451,148-140,451,208 forward strand.GRCh38:CM000685.2). Both 100 bp buffers are the first 100 bp of the corresponding 500 bp buffer (e.g., “buffer 1 (100 bp)” consists of the first 100 bp of “buffer 1 (500 bp”). The 30 bp buffers 1, 2, and 3, are sequential 30 bp sequences within “100 bp buffer 1”, downstream of the Mus musculus U7 pseudogene 8. These downstream sequences were selected due to the lack of any known regulatory sites or genes within or nearby to the sequence (using Gencode/Ensembl), in addition to lack of repetitive sequence, 40-60% GC content for total buffer, 40-60% GC content in the 20 bp region at both ends of the buffer, and minimal sequence complexity.

snRNA Sequences

The snRNA sequences of the disclosure can comprise any combination of esnRNA or snRNA features described herein.

In some embodiments, the snRNA comprises a targeting sequence that binds an SCN1A RNA sequence and a SL. In some embodiments, the SCN1A targeting sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 16-83. In some embodiments, the SL comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 84-124. In some embodiments, the SL comprises or consists of the nucleic acid sequence set forth in SEQ ID NO: 97.

In some embodiments, the snRNA comprises a targeting sequence that binds an SCN1A RNA sequence comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 16-83, and a SL comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 84-124. In some embodiments, the snRNA comprises a targeting sequence that binds an SCN1A RNA sequence comprising the nucleic acid sequence set forth in any one of SEQ ID NOs: 16-83, and the SL comprises the nucleic acid sequence set forth in SEQ ID NO: 97.

In some embodiments, the snRNA comprises a targeting sequence that binds an SCN1A RNA sequence comprising the nucleic acid sequence set forth in SEQ ID NO: 16, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 17, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 18, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 19, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 20, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 21, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 22, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 23, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 24, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 25, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 26, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 27, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 28, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 29, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 30, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 31, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 32, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 33, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 34, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 35, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 36, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 37, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 38, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 39, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 40, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 41, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 42, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 43, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 44, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 45, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 46, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 47, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 48, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 49, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 50, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 51, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 52, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 53, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 54, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 55, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 56, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 57, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 58, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 59, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 60, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 61, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 62, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 63, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 64, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 65, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 66, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 67, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 68, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 69, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 70, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 71, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 72, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 73, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 74, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 75, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 76, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 77, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 78, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 79, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 80, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 81, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 82, and the SL comprises the nucleic acid sequence set forth in SEQ ID NO: 97. In some embodiments, the snRNA comprises a targeting sequence that binds an SCN1A RNA sequence comprising the nucleic acid sequence set forth in SEQ ID NO: 83, and the SL comprises the nucleic acid sequence set forth in SEQ ID NO: 97.

In some embodiments, the SCN1A targeting sequence is positioned 5′ of the SL.

### Polynucleotides and Vectors

Also provided herein are polynucleotides and vectors (e.g., recombinant expression vectors) comprising the snRNA targeting SCN1A or SYNGAP1.

In some embodiments of the compositions and methods of the disclosure, a polynucleotide or vector comprises an snRNA system targeting SCN1A or SYNGAP1 is provided herein. In some embodiments of the compositions and methods of the disclosure, a polynucleotide or vector comprises an snRNA system targeting SCN1A is provided herein. In some embodiments of the compositions and methods of the disclosure, a polynucleotide or vector comprises an snRNA system targeting SYNGAP1 is provided herein. In some embodiments, the vector is a single or unitary vector.

Provided herein is a polynucleotide or vector comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 162-180. In some embodiments, the polynucleotide or vector comprises the nucleic acid sequence set forth in SEQ ID NO: 162. In some embodiments, the polynucleotide or vector comprises the nucleic acid sequence set forth in SEQ ID NO: 163. In some embodiments, the polynucleotide or vector comprises the nucleic acid sequence set forth in SEQ ID NO: 164. In some embodiments, the polynucleotide or vector comprises the nucleic acid sequence set forth in SEQ ID NO: 165. In some embodiments, the polynucleotide or vector comprises the nucleic acid sequence set forth in SEQ ID NO: 166. In some embodiments, the polynucleotide or vector comprises the nucleic acid sequence set forth in SEQ ID NO: 167. In some embodiments, the polynucleotide or vector comprises the nucleic acid sequence set forth in SEQ ID NO: 168. In some embodiments, the polynucleotide or vector comprises the nucleic acid sequence set forth in SEQ ID NO: 169. In some embodiments, the polynucleotide or vector comprises the nucleic acid sequence set forth in SEQ ID NO: 170. In some embodiments, the polynucleotide or vector comprises the nucleic acid sequence set forth in SEQ ID NO: 171. In some embodiments, the polynucleotide or vector comprises the nucleic acid sequence set forth in SEQ ID NO: 172. In some embodiments, the polynucleotide or vector comprises the nucleic acid sequence set forth in SEQ ID NO: 173. In some embodiments, the polynucleotide or vector comprises the nucleic acid sequence set forth in SEQ ID NO: 174. In some embodiments, the polynucleotide or vector comprises the nucleic acid sequence set forth in SEQ ID NO: 175. In some embodiments, the polynucleotide or vector comprises the nucleic acid sequence set forth in SEQ ID NO: 176. In some embodiments, the polynucleotide or vector comprises the nucleic acid sequence set forth in SEQ ID NO: 177. In some embodiments, the polynucleotide or vector comprises the nucleic acid sequence set forth in SEQ ID NO: 178. In some embodiments, the polynucleotide or vector comprises the nucleic acid sequence set forth in SEQ ID NO: 179. In some embodiments, the polynucleotide or vector comprises the nucleic acid sequence set forth in SEQ ID NO: 180.

In some embodiments, snRNA system is capable of targeting one or more SCN1A RNA sequences. In some aspects, the SCN1A RNA sequence is a SCN1A pre-mRNA sequence. In some embodiments, snRNA system is capable of targeting one or more SYNGAP1 RNA sequences. In some aspects, the SYNGAP1 RNA sequence is a SYNGAP1 pre-mRNA sequence. In some aspects, the snRNA systems are capable of targeting multiple (i.e., two or more) RNAs of interest. In some embodiments, the two or more RNAs of interest can be the same pre-mRNA molecule but different sequences within the pre-mRNA molecule.

One type of vector is a “plasmid,” which refers to a circular double stranded DNA loop into which DNA segments in addition to the nucleotide of interest can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentiviral (such as an integration-deficient lentiviral vector) or adeno-associated viral (AAV) vector. Vectors may be capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes they contain. Common expression vectors are often in the form of plasmids. In some embodiments, recombinant expression vectors comprise a nucleic acid provided herein such as e.g., an esnRNA in a form suitable for expression of an RNA molecule in a host cell. Recombinant expression vectors can include one or more regulatory elements, which may be selected on the basis of the host cells to be used for expression, that is operatively linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. In some embodiments, the regulatory element is a promoter described herein. In some embodiments, the regulatory element is a terminator provided herein.

Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., snRNAs, CRISPR transcripts, proteins, enzymes, mutant forms thereof, fusion proteins thereof, etc.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional snRNA promoters to express snRNAs.

In another embodiment, the vector configurations can comprise linker(s), signal sequence(s), and/or tag(s).

### Viral Vectors

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors.

In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAVrh8 vector, an AAV9 vector, an AAV.rh10 vector, a modified AAV.rh10 vector, an AAVrh.74, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, AAV-Tyr-Ser mutant vector, AAV-Tyr-Ser-Thr mutant vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism.

In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the vector further comprises one or more expression control elements operably linked to the polynucleotide. In some embodiments, the vector further comprises one or more selectable markers. In some embodiments, the vector has low toxicity. In some embodiments, the vector does not incorporate into the host genome, thereby having a low probability of causing insertional mutagenesis.

### Lentiviral Vectors

Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Adeno-Associated Virus Vectors

In some aspect, a vector described herein is an AAV viral vector. The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV (rAAV) genomes of the invention may comprise, consist essentially of, or consist of a nucleic acid molecule encoding at least one esnRNA and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

An AAV vector described herein may comprise, consist essentially of, or consist of one or more nucleic acid molecules and one or more AAV ITRs. In some aspects, the nucleic acid molecule encodes an esnRNA of the disclosure. Such AAV vectors can be replicated and packaged into infectious viral particles when present in a host cell that provides the functionality of rep and cap gene products, for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid encoding an esnRNA of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one terminator sequence. In some aspects, an AAV vector can comprise at least one polyA sequence. In some aspects, an AAV vector can comprise at least one linker sequence. In some aspects, an AAV vector can comprise at least one buffer sequence. In some aspects, an AAV vector of the disclosure can comprise at least one nuclear localization signal, or nuclear export signal and/or both. In some aspects, an AAV vector can comprise a first AAV ITR sequence, a promoter sequence, an snRNA sequence and/or esnRNA sequence, a terminator sequence and a second AAV ITR sequence. In some aspects, an AAV vector can comprise, in the 5′ to 3′ direction, a first AAV ITR sequence, a promoter sequence, an esnRNA sequence, a terminator sequence, and a second AAV ITR sequence. In some aspects, an AAV vector can comprise, in the 5′ to 3′ direction, a first AAV ITR sequence, a promoter sequence, an snRNA sequence, a terminator sequence, and a second AAV ITR sequence.

In some aspects, an AAV vector can comprise a first AAV ITR sequence, a first promoter sequence, a first snRNA sequence, a termination sequence, a second promoter sequence, second snRNA sequence, a second termination sequence and a second AAV ITR sequence. In some aspects, an AAV vector can comprise a first AAV ITR sequence, a first promoter sequence, a first snRNA sequence, a termination sequence, a second promoter sequence, a second snRNA sequence, a second termination sequence, a third promoter sequence, a third snRNA sequence, a third termination sequence, and a second AAV ITR sequence. In some aspects, an AAV vector can comprise a first AAV ITR sequence, a first promoter sequence, a first snRNA sequence, a termination, a second promoter sequence, a second snRNA sequence, a second termination sequence, a third promoter sequence, a third snRNA sequence, a third termination sequence, a fourth promoter sequence, a fourth snRNA sequence, a fourth termination sequence, and a second AAV ITR sequence.

In some embodiments of the compositions and methods of the disclosure, the viral vector comprises a sequence isolated or derived from an adeno-associated virus (AAV). In some embodiments, the viral vector comprises an ITR sequence or a capsid sequence that is isolated or derived from an AAV of serotype AAV1, AAV2, AAV3, AAV4, AAV5, AAV6, AAV7, AAV8, AAV9, AAV10, AAVrh10, AAV11 or AAV12. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the capsid is a ubiquitination resistant capsid. In another embodiment, the ubiquitination capsid is an AAV2 capsid comprising tyrosine (Y) and serine (S) mutations. In another embodiment, the AAV2 capsid comprises Y, S and threonine (T) mutations. In another embodiment, the AAV2 capsid includes, without limitation, AAV2 capsid mutants such as T455V, T491V, T550V, T659V, Y444+500+730F, and Y444+500+730F+T491V. In some embodiments, the viral vector is replication incompetent. In some embodiments, the viral vector is isolated or recombinant (rAAV). In some embodiments, the viral vector is self-complementary (scAAV). In some embodiments, the viral vector is single-stranded (ssAAV).

In some embodiments, the snRNAs provided herein are comprised within a single-stranded AAV (ssAAV). In some embodiments, the snRNAs provided herein are comprised within a self-complementary AAV (scAAV). The single-stranded nature of the parvoviral genome requires the use of cellular mechanisms to provide a complementary-strand for gene expression. This cellular recruitment activity is considered a rate-limiting factor in the efficiency of transduction and gene expression in parvoviruses and parvoviral particles. The use of an scAAV versus an ssAAV remedies this well-known issue by packaging both strands as a single duplex DNA molecule (or inverted repeat genome) that can fold into dsDNA as a result of a self-complementary viral genome sequence. In this regard, the requirement for DNA synthesis or base-pairing between multiple viral genomes is eliminated.

### AAV ITR Sequences

In some embodiments of the compositions and methods of the disclosure, an AAV inverted terminal repeat sequence can comprise any AAV ITR sequence known in the art. In some aspects, an AAV ITR sequence can comprise or consist of an AAV1 ITR sequence, an AAV2 ITR sequence, an AAV3 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAVrh10 ITR sequence, an AAV11 ITR sequence, an AAV12 ITR sequence, an AAV13 ITR sequence, or an AAVrh74 ITR sequence.

In some aspects the ITR sequence can comprise a modified AAV ITR sequence.

In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 158 or SEQ ID NO: 159.

In some embodiments, an AAV vector provided herein comprises a first and a second AAV ITR sequence. In some aspects, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 158 or SEQ ID NO: 159 and a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 158 or SEQ ID NO: 159. In some aspects the first AAV ITR sequence is positioned at the 5′ of an AAV vector. In some aspects the second AAV ITR sequence is positioned at the 3′ of an AAV vector.

In some aspects, a first AAV ITR sequence comprises the sequence set forth in SEQ ID NO: 158 or SEQ ID NO: 159. In some aspects, a second AAV ITR sequence comprises the sequence set forth in SEQ ID NO: 158 or SEQ ID NO: 159. In some embodiments, an AAV vector provided herein comprises a first AAV ITR sequence comprising the sequence set forth in SEQ ID NO: 158 and a second AAV ITR sequence comprising the sequence set forth in SEQ ID NO: 59. In some aspects the first AAV ITR sequence is positioned at the 5′ of an AAV vector. In some aspects the second AAV ITR sequence is positioned at the 3′ of an AAV vector.

In some embodiments of the compositions and methods of the disclosure, the viral vector comprises a sequence isolated or derived from an adeno-associated virus (AAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

snRNA Vector Constructs

Also provided herein are vector constructs targeting SCN1A comprising the snRNA constructs described herein. Also provided herein are vector constructs targeting SYNGAP1 comprising the snRNA constructs described herein.

An illustrative AAV vector of the disclosure targeting SCN1A exon 20N is A05290. The elements of A05290 are set forth in Table 6. In some aspects a nucleic acid sequence encoding AAV vector A05290 comprises SEQ ID NO: 162.

TABLE 6
A05290: scAAV-2x_mU7prom-SCN1A1z15/z6-mU7term_mU1prom-SCN1A1z15/z6-
mU1term; mouseloop with 5′ ISD and eSL; Nucleotide sequences of plasmid
elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z15/z6 | Aggggtaatacagtacccccaagttggagcaagattatcctatac (SEQ ID NO: 55) |
| SMOPT | AATTTTTGGAGca (SEQ ID NO: 160) |
| Stem loop | Ggttttctgacctccgtcggaaaacc (SEQ ID NO: 99) |
| Mouse U7 terminator | Cccaatttcactggtctacaatgaaagcaaaacagttctcttccccgctccccggtgtgt |
| | gagaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z15/z6 | Aggggtaatacagtacccccaagttggagcaagattatcctatac (SEQ ID NO: 55) |
| SMOPT | AATTTTTGGAGca (SEQ ID NO: 160) |
| Stem loop | Ggttttctgacctccgtcggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05290 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtaggggtaatacagtacccccaagttggagcaagattatcctatacAATTTTTGGAG
caggttttctgacctccgtcggaaaacccccaatttcactggtctacaatgaaagcaaaacagttctcttccccgctccccggtgtgtgagag
gggctttgatccttctctggtttcctaggaaacgcgtatgtgTTGTTCCTCTTAGTGTTAATTCACACTAAAGAC
TGTGCATCCGACTCCTACATTTATGAAAGTAAATGCCTGTTGTTAGAACAAAAAAGG
CTACAGAACAAAAAACAAAGCGAAATACCATCTGCTTTAGGTTCAGTGTGGTATTTT
CCCGCTGACAGGGAGGCGGGTTTTTGGGTACAGGAAACGAGTCACTATGGAGGCGG
TACTATGTAGATGAGAATTCAGGTGCAAACTGGGAAAAGCAACTGCTTCCAAATAT
TTGTGATTTTTACAGTGTAGTTTTGGAAAAACTCTTAGCCTACCAATTCTTCTAAGTG
TTTTAAAATGTGGGAGCCAGTACACATGAAGTTATAGAGTGTTTTAATGAGGCTTAA
ATATTTACCGTAACTATGAAATGCTACGCATATCATGCTGTTCAGGCTCCGTGGCCA
CGCAACTCggagtaggggtaatacagtacccccaagttggagcaagattatcctatacAATTTTTGGAGcaggttttctga
cctccgtcggaaaaccGTTTACTTGGTTTTAAAAATAGCTTGCACTAGCGATACGGAATATGG
TTATTAGGTTTGTTAGGCATCATGTCGTGTCTTACTATAGAAAAATAACGTAGTGTTC
ATTTTAGCCTGCCTGTATGTGTTAATTTGTCCTTATTGCGCATTGTTCTTGTTAAGTCT
TCTGTAAGGAGTTGCGGGTTTCAAACTGTCAGTCTGAGAGCAGAATTCGATATCTAG
ATCTCGAGGTAACCACGTGCGGACCCAACGGCCGCaggaacccctagtgatggagttggccactccctct
ctgcgcgctcgctcgctcactgaggccgggcgaccaaaggtcgcccgacgcccgggctttgcccgggcggcctcagtgagcgagcga
gcgcgcagctgcctgcagg (SEQ ID NO: 162).

Another illustrative AAV vector of the disclosure targeting SCN1A exon 20N is A05291. The elements of A05291 are set forth in Table 7. In some aspects a nucleic acid sequence encoding AAV vector A05291 comprises SEQ ID NO: 163.

TABLE 7
A05291: scAAV-2x_mU7prom-SCN1A1z11/z15-mU7term_mU1prom-SCN1A1z11/z15-
mU1term; mouseloop with 5′ ISD and eSL; Nucleotide sequences of plasmid
elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z11/z15 | Gttggagcaagattatcctaaggggtaatacagtaccc (SEQ ID NO: 57) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop | ggttttctgacctccgtcggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05291 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtgttggagcaagattatcctaaggggtaatacagtacccAATTTTTGGAGcaggtttt
ctgacctccgtcggaaaacccccaatttcactggtctacaatgaaagcaaaacagttctcttccccgctccccggtgtgtgagaggggctttg
atccttctctggtttcctaggaaacgcgtatgtgTTGTTCCTCTTAGTGTTAATTCACACTAAAGACTGTGC
ATCCGACTCCTACATTTATGAAAGTAAATGCCTGTTGTTAGAACAAAAAAGGCTACA
GAACAAAAAACAAAGCGAAATACCATCTGCTTTAGGTTCAGTGTGGTATTTTCCCGC
TGACAGGGAGGCGGGTTTTTGGGTACAGGAAACGAGTCACTATGGAGGCGGTACTA
TGTAGATGAGAATTCAGGTGCAAACTGGGAAAAGCAACTGCTTCCAAATATTTGTG
ATTTTTACAGTGTAGTTTTGGAAAAACTCTTAGCCTACCAATTCTTCTAAGTGTTTTA
AAATGTGGGAGCCAGTACACATGAAGTTATAGAGTGTTTTAATGAGGCTTAAATATT
TACCGTAACTATGAAATGCTACGCATATCATGCTGTTCAGGCTCCGTGGCCACGCAA
CTCggagtgttggagcaagattatcctaaggggtaatacagtacccAATTTTTGGAGcaggttttctgacctccgtcggaaaac
cGTTTACTTGGTTTTAAAAATAGCTTGCACTAGCGATACGGAATATGGTTATTAGGTT
TGTTAGGCATCATGTCGTGTCTTACTATAGAAAAATAACGTAGTGTTCATTTTAGCCT
GCCTGTATGTGTTAATTTGTCCTTATTGCGCATTGTTCTTGTTAAGTCTTCTGTAAGG
AGTTGCGGGTTTCAAACTGTCAGTCTGAGAGCAGAATTCGATATCTAGATCTCGAGG
TAACCACGTGCGGACCCAACGGCCGCaggaacccctagtgatggagttggccactccctctctgcgcgctcgctc
gctcactgaggccgggcgaccaaaggtcgcccgacgcccgggctttgcccgggcggcctcagtgagcgagcgagcgcgcagctgcct
gcagg (SEQ ID NO: 163).

Another illustrative AAV vector of the disclosure targeting SCN1A exon 20N is A05378. The elements of A05378 are set forth in Table 8. In some aspects a nucleic acid sequence encoding AAV vector A05378 comprises SEQ ID NO: 164.

TABLE 8
A05378: scAAV-2x_mU7prom-SCN1A1z12/z6-mU7term_mU1prom-SCN1A1z12/z6-
mU1term; mouseloop with 5′ ISD and eSL; Nucleotide sequences of plasmid
elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z12/z6 | Aaaggggtaatacagtacccccaagttggagcaagattatcctatac (SEQ NO: 54) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z12/z6 | aaaggggtaatacagtacccccaagttggagcaagattatcctatac (SEQ NO: 54) |
| SMOPT | AATTTTTGGAGca (SEQ ID NO: 160) |
| Stem loop | ggttttctgacctccgtoggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05378 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtaaaggggtaatacagtacccccaagttggagcaagattatcctatacAATTTTTGGA
Gcaggttttctgacctccgtcggaaaacccccaatttcactggtctacaatgaaagcaaaacagttctcttccccgctccccggtgtgtgaga
ggggctttgatccttctctggtttcctaggaaacgcgtatgtgTTGTTCCTCTTAGTGTTAATTCACACTAAAGA
CTGTGCATCCGACTCCTACATTTATGAAAGTAAATGCCTGTTGTTAGAACAAAAAAG
GCTACAGAACAAAAAACAAAGCGAAATACCATCTGCTTTAGGTTCAGTGTGGTATTT
TCCCGCTGACAGGGAGGCGGGTTTTTGGGTACAGGAAACGAGTCACTATGGAGGCG
GTACTATGTAGATGAGAATTCAGGTGCAAACTGGGAAAAGCAACTGCTTCCAAATA
TTTGTGATTTTTACAGTGTAGTTTTGGAAAAACTCTTAGCCTACCAATTCTTCTAAGT
GTTTTAAAATGTGGGAGCCAGTACACATGAAGTTATAGAGTGTTTTAATGAGGCTTA
AATATTTACCGTAACTATGAAATGCTACGCATATCATGCTGTTCAGGCTCCGTGGCC
ACGCAACTCggagtaaaggggtaatacagtacccccaagttggagcaagattatcctatacAATTTTTGGAGcaggtttt
ctgacctccgtcggaaaaccGTTTACTTGGTTTTAAAAATAGCTTGCACTAGCGATACGGAATA
TGGTTATTAGGTTTGTTAGGCATCATGTCGTGTCTTACTATAGAAAAATAACGTAGT
GTTCATTTTAGCCTGCCTGTATGTGTTAATTTGTCCTTATTGCGCATTGTTCTTGTTAA
GTCTTCTGTAAGGAGTTGCGGGTTTCAAACTGTCAGTCTGAGAGCAGAATTCGATAT
CTAGATCTCGAGGTAACCACGTGCGGACCCAACGGCCGCaggaacccctagtgatggagttggccac
tccctctctgcgcgctcgctcgctcactgaggccgggcgaccaaaggtcgcccgacgcccgggctttgcccgggggcctcagtgagcg
agcgagcgcgcagctgcctgcagg (SEQ ID NO: 164).

Another illustrative AAV vector of the disclosure targeting SCN1A exon 20N is A05379. The elements of A05379 are set forth in Table 9. In some aspects a nucleic acid sequence encoding AAV vector A05379 comprises SEQ ID NO: 165.

TABLE 9
A05379: scAAV-2x_mU7prom-SCN1A1z33/z15-mU7term_mU1prom-SCN1A1z33/z15-
mU1term; mouseloop with 5′ ISD and eSL; Nucleotide sequences of plasmid
elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z33/z15 | AGTTGGAGCAAGATTATCCTAaggggtaatacagtaccc (SEQ NO: 74) |
| SMOPT | AATTTTTGGAGca (SEQ ID NO: 160) |
| Stem loop | ggttttctgacctccgteggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z33/z15 | AGTTGGAGCAAGATTATCCTAaggggtaatacagtaccc (SEQ NO: 74) |
| SMOPT | AATTTTTGGAGca (SEQ ID NO: 160) |
| Stem loop | ggttttctgacctccgteggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05379 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtAGTTGGAGCAAGATTATCCTAaggggtaatacagtacccAATTTT
TGGAGcaggttttctgacctccgtcggaaaacccccaatttcactggtctacaatgaaagcaaaacagttctcttccccgctccccggtgt
gtgagaggggctttgatccttctctggtttcctaggaaacgcgtatgtgTTGTTCCTCTTAGTGTTAATTCACACTA
AAGACTGTGCATCCGACTCCTACATTTATGAAAGTAAATGCCTGTTGTTAGAACAAA
AAAGGCTACAGAACAAAAAACAAAGCGAAATACCATCTGCTTTAGGTTCAGTGTGG
TATTTTCCCGCTGACAGGGAGGCGGGTTTTTGGGTACAGGAAACGAGTCACTATGGA
GGCGGTACTATGTAGATGAGAATTCAGGTGCAAACTGGGAAAAGCAACTGCTTCCA
AATATTTGTGATTTTTACAGTGTAGTTTTGGAAAAACTCTTAGCCTACCAATTCTTCT
AAGTGTTTTAAAATGTGGGAGCCAGTACACATGAAGTTATAGAGTGTTTTAATGAGG
CTTAAATATTTACCGTAACTATGAAATGCTACGCATATCATGCTGTTCAGGCTCCGT
GGCCACGCAACTCggagtAGTTGGAGCAAGATTATCCTAaggggtaatacagtacccAATTTTTG
GAGcaggttttctgacctccgtcggaaaaccGTTTACTTGGTTTTAAAAATAGCTTGCACTAGCGATA
CGGAATATGGTTATTAGGTTTGTTAGGCATCATGTCGTGTCTTACTATAGAAAAATA
ACGTAGTGTTCATTTTAGCCTGCCTGTATGTGTTAATTTGTCCTTATTGCGCATTGTT
CTTGTTAAGTCTTCTGTAAGGAGTTGCGGGTTTCAAACTGTCAGTCTGAGAGCAGAA
TTCGATATCTAGATCTCGAGGTAACCACGTGCGGACCCAACGGCCGCaggaacccctagtgat
ggagttggccactccctctctgcgcgctcgctcgctcactgaggccgggcgaccaaaggtcgcccgacgcccgggctttgcccgggcgg
cctcagtgagcgagcgagcgcgcagctgcctgcagg (SEQ ID NO: 165).

Another illustrative AAV vector of the disclosure targeting SCN1A exon 20N is A05380. The elements of A05380 are set forth in Table 10. In some aspects a nucleic acid sequence encoding AAV vector A05380 comprises SEQ ID NO: 166.

TABLE 10
A05380: scAAV-2x_mU7prom-SCN1A1z15/z33-mU7term_mU1prom-SCN1A1z15/z33-
mU1term; mouseloop with 5′ ISD and eSL; Nucleotide sequences of plasmid
elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z15/z33 | aggggtaatacagtacccAGTTGGAGCAAGATTATCCTA (SEQ ID NO: 75) |
| SMOPT | AATTTTTGGAGca (SEQ ID NO: 160) |
| Stem loop | ggttttctgacctccgteggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z15/z33 | aggggtaatacagtacccAGTTGGAGCAAGATTATCCTA (SEQ ID NO: 75) |
| SMOPT | AATTTTTGGAGca (SEQ ID NO: 160) |
| Stem loop | ggttttctgacctccgtcggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05380 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtaggggtaatacagtacccAGTTGGAGCAAGATTATCCTAAATTTT
TGGAGcaggttttctgacctccgtcggaaaacccccaatttcactggtctacaatgaaagcaaaacagttctcttccccgctccccggtgt
gtgagaggggctttgatccttctctggtttcctaggaaacgcgtatgtgTTGTTCCTCTTAGTGTTAATTCACACTA
AAGACTGTGCATCCGACTCCTACATTTATGAAAGTAAATGCCTGTTGTTAGAACAAA
AAAGGCTACAGAACAAAAAACAAAGCGAAATACCATCTGCTTTAGGTTCAGTGTGG
TATTTTCCCGCTGACAGGGAGGCGGGTTTTTGGGTACAGGAAACGAGTCACTATGGA
GGCGGTACTATGTAGATGAGAATTCAGGTGCAAACTGGGAAAAGCAACTGCTTCCA
AATATTTGTGATTTTTACAGTGTAGTTTTGGAAAAACTCTTAGCCTACCAATTCTTCT
AAGTGTTTTAAAATGTGGGAGCCAGTACACATGAAGTTATAGAGTGTTTTAATGAGG
CTTAAATATTTACCGTAACTATGAAATGCTACGCATATCATGCTGTTCAGGCTCCGT
GGCCACGCAACTCggagtaggggtaatacagtacccAGTTGGAGCAAGATTATCCTAAATTTTTG
GAGcaggttttctgacctccgtcggaaaaccGTTTACTTGGTTTTAAAAATAGCTTGCACTAGCGATA
CGGAATATGGTTATTAGGTTTGTTAGGCATCATGTCGTGTCTTACTATAGAAAAATA
ACGTAGTGTTCATTTTAGCCTGCCTGTATGTGTTAATTTGTCCTTATTGCGCATTGTT
CTTGTTAAGTCTTCTGTAAGGAGTTGCGGGTTTCAAACTGTCAGTCTGAGAGCAGAA
TTCGATATCTAGATCTCGAGGTAACCACGTGCGGACCCAACGGCCGCaggaacccctagtgat
ggagttggccactccctctctgcgcgctcgctcgctcactgaggccgggcgaccaaaggtcgcccgacgcccgggctttgcccgggcgg
cctcagtgagcgagcgagcgcgcagctgcctgcagg (SEQ ID NO: 166).

Another illustrative AAV vector of the disclosure targeting SCN1A exon 20N is A05381. The elements of A05381 are set forth in Table 11. In some aspects a nucleic acid sequence encoding AAV vector A05381 comprises SEQ ID NO: 167.

TABLE 11
A05381: scAAV-2x_mU7prom-SCN1A1z33/z36-mU7term_mU1prom-
SCN1A1z33/z36-mU1term; mouseloop with 5′ ISD and eSL; Nucleotide sequences
of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z33/z36 | AGTTGGAGCAAGATTATCCTAAGTACCCATAATAAAGGG (SEQ ID NO: 88) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z33/z36 | AGTTGGAGCAAGATTATCCTAAGTACCCATAATAAAGGG (SEQ ID NO: 88) |
| SMOPT | AATTTTTGGAGca (SEQ ID NO: 160) |
| Stem loop | ggttttctgacctccgtcggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05381 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtAGTTGGAGCAAGATTATCCTAAGTACCCATAATAAAGG
GAATTTTTGGAGcaggttttctgacctccgtcggaaaacccccaatttcactggtctacaatgaaagcaaaacagttctcttccccg
ctccccggtgtgtgagaggggctttgatccttctctggtttcctaggaaacgcgtatgtgTTGTTCCTCTTAGTGTTAATTC
ACACTAAAGACTGTGCATCCGACTCCTACATTTATGAAAGTAAATGCCTGTTGTTAG
AACAAAAAAGGCTACAGAACAAAAAACAAAGCGAAATACCATCTGCTTTAGGTTCA
GTGTGGTATTTTCCCGCTGACAGGGAGGCGGGTTTTTGGGTACAGGAAACGAGTCAC
TATGGAGGCGGTACTATGTAGATGAGAATTCAGGTGCAAACTGGGAAAAGCAACTG
CTTCCAAATATTTGTGATTTTTACAGTGTAGTTTTGGAAAAACTCTTAGCCTACCAAT
TCTTCTAAGTGTTTTAAAATGTGGGAGCCAGTACACATGAAGTTATAGAGTGTTTTA
ATGAGGCTTAAATATTTACCGTAACTATGAAATGCTACGCATATCATGCTGTTCAGG
CTCCGTGGCCACGCAACTCggagtAGTTGGAGCAAGATTATCCTAAGTACCCATAATA
AAGGGAATTTTTGGAGcaggttttctgacctccgtcggaaaaccGTTTACTTGGTTTTAAAAATAGCT
TGCACTAGCGATACGGAATATGGTTATTAGGTTTGTTAGGCATCATGTCGTGTCTTA
CTATAGAAAAATAACGTAGTGTTCATTTTAGCCTGCCTGTATGTGTTAATTTGTCCTT
ATTGCGCATTGTTCTTGTTAAGTCTTCTGTAAGGAGTTGCGGGTTTCAAACTGTCAGT
CTGAGAGCAGAATTCGATATCTAGATCTCGAGGTAACCACGTGCGGACCCAACGGC
CGCaggaacccctagtgatggagttggccactccctctctgcgcgctcgctcgctcactgaggccgggcgaccaaaggtcgcccgacg
cccgggctttgcccgggcggcctcagtgagcgagcgagcgcgcagctgcctgcagg (SEQ ID NO: 167).

Another illustrative AAV vector of the disclosure targeting SCN1A exon 20N is A05382. The elements of A05382 are set forth in Table 12. In some aspects a nucleic acid sequence encoding AAV vector A05382 comprises SEQ ID NO: 168.

TABLE 12
A05382: scAAV-2x_mU7prom-SCN1A1z36/z33-mU7term_mU1prom-
SCN1A1z36/z33-mU1term; mouseloop with 5′ ISD and eSL; Nucleotide sequences
of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z36/z33 | AGTACCCATAATAAAGGGAGTTGGAGCAAGATTATCCTA (SEQ ID NO: 81) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z36/z33 | AGTACCCATAATAAAGGGAGTTGGAGCAAGATTATCCTA (SEQ ID NO: 81) |
| SMOPT | AATTTTTGGAGca (SEQ ID NO: 160) |
| Stem loop | ggttttctgacctccgteggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3?ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05382 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtAGTACCCATAATAAAGGGAGTTGGAGCAAGATTATCCT
AAATTTTTGGAGcaggttttctgacctccgtcggaaaacccccaatttcactggtctacaatgaaagcaaaacagttctcttccccg
ctccccggtgtgtgagaggggctttgatccttctctggtttcctaggaaacgcgtatgtgTTGTTCCTCTTAGTGTTAATTC
ACACTAAAGACTGTGCATCCGACTCCTACATTTATGAAAGTAAATGCCTGTTGTTAG
AACAAAAAAGGCTACAGAACAAAAAACAAAGCGAAATACCATCTGCTTTAGGTTCA
GTGTGGTATTTTCCCGCTGACAGGGAGGCGGGTTTTTGGGTACAGGAAACGAGTCAC
TATGGAGGCGGTACTATGTAGATGAGAATTCAGGTGCAAACTGGGAAAAGCAACTG
CTTCCAAATATTTGTGATTTTTACAGTGTAGTTTTGGAAAAACTCTTAGCCTACCAAT
TCTTCTAAGTGTTTTAAAATGTGGGAGCCAGTACACATGAAGTTATAGAGTGTTTTA
ATGAGGCTTAAATATTTACCGTAACTATGAAATGCTACGCATATCATGCTGTTCAGG
CTCCGTGGCCACGCAACTCggagtAGTACCCATAATAAAGGGAGTTGGAGCAAGATTA
TCCTAAATTTTTGGAGcaggttttctgacctccgtcggaaaaccGTTTACTTGGTTTTAAAAATAGCTT
GCACTAGCGATACGGAATATGGTTATTAGGTTTGTTAGGCATCATGTCGTGTCTTAC
TATAGAAAAATAACGTAGTGTTCATTTTAGCCTGCCTGTATGTGTTAATTTGTCCTTA
TTGCGCATTGTTCTTGTTAAGTCTTCTGTAAGGAGTTGCGGGTTTCAAACTGTCAGTC
TGAGAGCAGAATTCGATATCTAGATCTCGAGGTAACCACGTGCGGACCCAACGGCC
GCaggaacccctagtgatggagttggccactccctctctgcgcgctcgctcgctcactgaggccgggcgaccaaaggtcgcccgacgc
ccgggctttgcccgggcggcctcagtgagcgagcgagcgcgcagctgcctgcagg (SEQ ID NO: 168)

Another illustrative AAV vector of the disclosure targeting SCN1A exon 20N is A05383. The elements of A05383 are set forth in Table 13. In some aspects a nucleic acid sequence encoding AAV vector A05383 comprises SEQ ID NO: 169.

TABLE 13
A05383: scAAV-2x_mU7prom-SCN1A1z21/z33-mU7term_mU1prom- SCNIA1z21/z33-mU1term;
mouseloop with 5′ ISD and eSL; Nucleotide sequences of plasmid elements in order
N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z21/z33 | agtcacagtgcaaggattaaaggtagAGTTGGAGCAAGATTATCCT |
| | A (SEQ ID NO: 82) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z21/z33 | agtcacagtgcaaggattaaaggtagAGTTGGAGCAAGATTATCCT |
| | A (SEQ ID NO: 82) |
| SMOPT | AATTTTTGGAGca (SEQ ID NO: 160) |
| Stem loop | ggttttctgacctccgtoggaaaacc (SEQ ID NO: 99) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05383 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtagtcacagtgcaaggattaaaggtagAGTTGGAGCAAGATTATCCTAA
ATTTTTGGAGcaggttttctgacctccgtcggaaaacccccaatttcactggtctacaatgaaagcaaaacagttctcttccccgctc
cccggtgtgtgagaggggctttgatccttctctggtttcctaggaaacgcgtatgtgTTGTTCCTCTTAGTGTTAATTCA
CACTAAAGACTGTGCATCCGACTCCTACATTTATGAAAGTAAATGCCTGTTGTTAGA
ACAAAAAAGGCTACAGAACAAAAAACAAAGCGAAATACCATCTGCTTTAGGTTCAG
TGTGGTATTTTCCCGCTGACAGGGAGGCGGGTTTTTGGGTACAGGAAACGAGTCACT
ATGGAGGCGGTACTATGTAGATGAGAATTCAGGTGCAAACTGGGAAAAGCAACTGC
TTCCAAATATTTGTGATTTTTACAGTGTAGTTTTGGAAAAACTCTTAGCCTACCAATT
CTTCTAAGTGTTTTAAAATGTGGGAGCCAGTACACATGAAGTTATAGAGTGTTTTAA
TGAGGCTTAAATATTTACCGTAACTATGAAATGCTACGCATATCATGCTGTTCAGGC
TCCGTGGCCACGCAACTCggagtagtcacagtgcaaggattaaaggtagAGTTGGAGCAAGATTATCC
TAAATTTTTGGAGcaggttttctgacctccgtcggaaaaccGTTTACTTGGTTTTAAAAATAGCTTGC
ACTAGCGATACGGAATATGGTTATTAGGTTTGTTAGGCATCATGTCGTGTCTTACTA
TAGAAAAATAACGTAGTGTTCATTTTAGCCTGCCTGTATGTGTTAATTTGTCCTTATT
GCGCATTGTTCTTGTTAAGTCTTCTGTAAGGAGTTGCGGGTTTCAAACTGTCAGTCTG
AGAGCAGAATTCGATATCTAGATCTCGAGGTAACCACGTGCGGACCCAACGGCCGC
aggaacccctagtgatggagttggccactccctctctgcgcgctcgctcgctcactgaggccgggcgaccaaaggtcgcccgacgcccg
ggctttgcccgggcggcctcagtgagcgagcgagcgcgcagctgcctgcagg (SEQ ID NO: 169).

Another illustrative AAV vector of the disclosure targeting SCN1A exon 20N is A05384. The elements of A05384 are set forth in Table 14. In some aspects a nucleic acid sequence encoding AAV vector A05384 comprises SEQ ID NO: 170.

TABLE 14
A05384: scAAV-2x_mU7prom-SCN1A1z33/z21-mU7term_mU1prom- SCNIA1z33/z21-mU1term;
mouseloop with 5′ ISD and eSL; Nucleotide sequences of plasmid elements in order
N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| ISD extra nt | aa |
| ISD | ggagt |
| Fusion spacer z33/z21 | AGTTGGAGCAAGATTATCCTAagtcacagtgcaaggattaaaggta |
| | g (SEQ ID NO: 83) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| ISD | ggagt |
| Fusion spacer z33/z21 | AGTTGGAGCAAGATTATCCTAagtcacagtgcaaggattaaaggta |
| | g (SEQ ID NO: 83) |
| SMOPT | AATTTTTGGAGca (SEQ ID NO: 160) |
| Stem loop | ggttttctgacctccgtcggaaaacc (SEQ ID NO: 99) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05384 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataagg
aactgtgctttgtgattcacatatcagtggaggggtgtggaaatggcaccttgatctcaccctcatcgaaagtggagttgatgtcctTccctgg
ctcgctacagacgcacttccgcaaggagtAGTTGGAGCAAGATTATCCTAagtcacagtgcaaggattaaaggtagA
ATTTTTGGAGcaggttttctgacctccgtcggaaaacccccaatttcactggtctacaatgaaagcaaaacagttctcttccccgctc
cccggtgtgtgagaggggctttgatccttctctggtttcctaggaaacgcgtatgtgTTGTTCCTCTTAGTGTTAATTCA
CACTAAAGACTGTGCATCCGACTCCTACATTTATGAAAGTAAATGCCTGTTGTTAGA
ACAAAAAAGGCTACAGAACAAAAAACAAAGCGAAATACCATCTGCTTTAGGTTCAG
TGTGGTATTTTCCCGCTGACAGGGAGGCGGGTTTTTGGGTACAGGAAACGAGTCACT
ATGGAGGCGGTACTATGTAGATGAGAATTCAGGTGCAAACTGGGAAAAGCAACTGC
TTCCAAATATTTGTGATTTTTACAGTGTAGTTTTGGAAAAACTCTTAGCCTACCAATT
CTTCTAAGTGTTTTAAAATGTGGGAGCCAGTACACATGAAGTTATAGAGTGTTTTAA
TGAGGCTTAAATATTTACCGTAACTATGAAATGCTACGCATATCATGCTGTTCAGGC
TCCGTGGCCACGCAACTCggagtAGTTGGAGCAAGATTATCCTAagtcacagtgcaaggattaaagg
tagAATTTTTGGAGcaggttttctgacctccgtcggaaaaccGTTTACTTGGTTTTAAAAATAGCTTGCA
CTAGCGATACGGAATATGGTTATTAGGTTTGTTAGGCATCATGTCGTGTCTTACTAT
AGAAAAATAACGTAGTGTTCATTTTAGCCTGCCTGTATGTGTTAATTTGTCCTTATTG
CGCATTGTTCTTGTTAAGTCTTCTGTAAGGAGTTGCGGGTTTCAAACTGTCAGTCTGA
GAGCAGAATTCGATATCTAGATCTCGAGGTAACCACGTGCGGACCCAACGGCCGCag
gaacccctagtgatggagttggccactccctctctgcgcgctcgctcgctcactgaggccgggcgaccaaaggtcgcccgacgcccggg
ctttgcccgggcggcctcagtgagcgagcgagcgcgcagctgcctgcagg (SEQ ID NO: 170).

Another illustrative AAV vector of the disclosure targeting SCN1A exon 20N is A05865. The elements of A05865 are set forth in Table 15. In some aspects a nucleic acid sequence encoding AAV vector A05865 comprises SEQ ID NO: 171.

TABLE 15
A05865: scAAV-2x_mU7prom-SCNIA1z15/z6-mU7term_mU1prom- SCNIA1z15/z6-
mU1term; (mouseloop without 5′ ISD and with eSL) (equivalent to synthetic SR220517);
Nucleotide sequences of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop | ggttttctgacctccgtcggaaaacc (SEQ ID NO: 99) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05865 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaaaggggtaatacagtacccccaagttggagcaagattatcctatacAATTTTTGGAGc
aggttttctgacctccgtcggaaaacccccaatttcactggtctacaatgaaagcaaaacagttctcttccccgctccccggtgtgtg
agaggggctttgatccttctctggtttcctaggaaacgcgtatgtgTTGTTCCTCTTAGTGTTAATTCACACTAAAGACTGTG
CATCCGACTCCTACATTTATGAAAGTAAATGCCTGTTGTTAGAACAAAAAAGGCTAC
AGAACAAAAAACAAAGCGAAATACCATCTGCTTTAGGTTCAGTGTGGTATTTTCCCG
CTGACAGGGAGGCGGGTTTTTGGGTACAGGAAACGAGTCACTATGGAGGCGGTACT
ATGTAGATGAGAATTCAGGTGCAAACTGGGAAAAGCAACTGCTTCCAAATATTTGT
GATTTTTACAGTGTAGTTTTGGAAAAACTCTTAGCCTACCAATTCTTCTAAGTGTTTT
AAAATGTGGGAGCCAGTACACATGAAGTTATAGAGTGTTTTAATGAGGCTTAAATA
TTTACCGTAACTATGAAATGCTACGCATATCATGCTGTTCAGGCTCCGTGGCCACGC
AACTCaggggtaatacagtacccccaagttggagcaagattatcctatacAATTTTTGGAGcaggttttctgacctccgtcgg
aaaaccGTTTACTTGGTTTTAAAAATAGCTTGCACTAGCGATACGGAATATGGTTATTA
GGTTTGTTAGGCATCATGTCGTGTCTTACTATAGAAAAATAACGTAGTGTTCATTTTA
GCCTGCCTGTATGTGTTAATTTGTCCTTATTGCGCATTGTTCTTGTTAAGTCTTCTGTA
AGGAGTTGCGGGTTTCAAACTGTCAGTCTGAGAGCAGAATTCGATATCTAGATCTCG
AGGTAACCACGTGCGGACCCAACGGCCGCaggaacccctagtgatggagttggccactccctctctgcgcgct

Another illustrative AAV vector of the disclosure targeting SCN1A exon 20N is A05866. The elements of A05866 are set forth in Table 16. In some aspects a nucleic acid sequence encoding AAV vector A05866 comprises SEQ ID NO: 172.

TABLE 16
A05866: scAAV-2x_mU7prom-SCN1A1z11/z15-mU7term_mU1prom-
SCN1A1z11/z15-mU1term; (mouseloop without 5′ ISD, and with eSL) (equivalent to synthetic
SR220518); Nucleotide sequences of plasmid elements in order N-terminal to C-terminal;
Nucleotide sequences of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop | ggttttctgacctccgtcggaaaacc (SEQ ID NO: 99) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop | ggttttctgacctccgtoggaaaacc (SEQ ID NO: 99) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05866 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaagttggagcaagattatcctaaggggtaatacagtacccAATTTTTGGAgcaggtttt
ctgacctccgtcggaaaacccccaatttcactggtctacaatgaaagcaaaacagttctcttccccgctccccggtgtgtgagagggg
ctttgatccttctctggtttcctaggaaacgcgtatgtgTTGTTCCTCTTAGTGTTAATTCACACTAAAGACTGTGCATCC
GACTCCTACATTTATGAAAGTAAATGCCTGTTGTTAGAACAAAAAAGGCTACAGAA
CAAAAAACAAAGCGAAATACCATCTGCTTTAGGTTCAGTGTGGTATTTTCCCGCTGA
CAGGGAGGCGGGTTTTTGGGTACAGGAAACGAGTCACTATGGAGGCGGTACTATGT
AGATGAGAATTCAGGTGCAAACTGGGAAAAGCAACTGCTTCCAAATATTTGTGATTT
TTACAGTGTAGTTTTGGAAAAACTCTTAGCCTACCAATTCTTCTAAGTGTTTTAAAAT
GTGGGAGCCAGTACACATGAAGTTATAGAGTGTTTTAATGAGGCTTAAATATTTACC
GTAACTATGAAATGCTACGCATATCATGCTGTTCAGGCTCCGTGGCCACGCAACTCgt
tggagcaagattatcctaaggggtaatacagtacccAATTTTTGGAGcaggttttctgacctccgtcggaaaaccGTTTACT
TGGTTTTAAAAATAGCTTGCACTAGCGATACGGAATATGGTTATTAGGTTTGTTAGG
CATCATGTCGTGTCTTACTATAGAAAAATAACGTAGTGTTCATTTTAGCCTGCCTGTA
TGTGTTAATTTGTCCTTATTGCGCATTGTTCTTGTTAAGTCTTCTGTAAGGAGTTGCG
GGTTTCAAACTGTCAGTCTGAGAGCAGAATTCGATATCTAGATCTCGAGGTAACCAC
GTGCGGACCCAACGGCCGCaggaacccctagtgatggagttggccactccctctctgcgcgctcgctcgctcactgagg
ccgggcgaccaaaggtcgcccgacgcccgggctttgcccgggcggcctcagtgagcgagcgagcgcgcagctgcctgcagg (SEQ
ID NO: 172).

Another illustrative AAV vector of the disclosure targeting SCN1A exon 20N is A05867. The elements of A05867 are set forth in Table 17. In some aspects a nucleic acid sequence encoding AAV vector A05867 comprises SEQ ID NO: 173.

TABLE 17
A05867: scAAV-2x_mU7prom-SCNIA1z15/z6 mU7term_mU1prom-SCNIA1z15/z6-
mU1term; (mouseloop without 5′ ISD and without eSL) (equivalent to synthetic SR220520);
Nucleotide sequences of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/ long) | Ggttttctgacttcggtcggaaaacccct (SEQ ID NO: 119) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/ long) | Ggttttctgacttcggtcggaaaacccct (SEQ ID NO: 119) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05867 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaaaggggtaatacagtacccccaagttggagcaagattatcctatacAATTTTTGGAGc
aggttttctgacttcggtcggaaaacccctcccaatttcactggtctacaatgaaagcaaaacagttctcttccccgctccccggtgt
gtgagaggggctttgatccttctctggtttcctaggaaacgcgtatgtgTTGTTCCTCTTAGTGTTAATTCACACTAAAGACTG
TGCATCCGACTCCTACATTTATGAAAGTAAATGCCTGTTGTTAGAACAAAAAAGGCT
ACAGAACAAAAAACAAAGCGAAATACCATCTGCTTTAGGTTCAGTGTGGTATTTTCC
CGCTGACAGGGAGGCGGGTTTTTGGGTACAGGAAACGAGTCACTATGGAGGCGGTA
CTATGTAGATGAGAATTCAGGTGCAAACTGGGAAAAGCAACTGCTTCCAAATATTT
GTGATTTTTACAGTGTAGTTTTGGAAAAACTCTTAGCCTACCAATTCTTCTAAGTGTT
TTAAAATGTGGGAGCCAGTACACATGAAGTTATAGAGTGTTTTAATGAGGCTTAAAT
ATTTACCGTAACTATGAAATGCTACGCATATCATGCTGTTCAGGCTCCGTGGCCACG
CAACTCaggggtaatacagtacccccaagttggagcaagattatcctatacAATTTTTGGAGcaggttttctgacttcggtcg
gaaaacccctGTTTACTTGGTTTTAAAAATAGCTTGCACTAGCGATACGGAATATGGTTAT
TAGGTTTGTTAGGCATCATGTCGTGTCTTACTATAGAAAAATAACGTAGTGTTCATTT
TAGCCTGCCTGTATGTGTTAATTTGTCCTTATTGCGCATTGTTCTTGTTAAGTCTTCTG
TAAGGAGTTGCGGGTTTCAAACTGTCAGTCTGAGAGCAGAATTCGATATCTAGATCT
CGAGGTAACCACGTGCGGACCCAACGGCCGCaggaacccctagtgatggagttggccactccctctctgcgc
gctcgctcgctcactgaggccgggcgaccaaaggtcgcccgacgcccgggctttgcccgggggcctcagtgagcgagcgagcgcgc
agctgcctgcagg (SEQ ID NO: 173).

Another illustrative AAV vector of the disclosure targeting SCN1A exon 20N is A05868. The elements of A05868 are set forth in Table 18. In some aspects a nucleic acid sequence encoding AAV vector A05868 comprises SEQ ID NO: 174.

TABLE 18
A05868: scAAV-2x_mU7prom-SCNIA1z11/z15-mU7term_mU1prom-
SCN1A1z11/z15-mU1term; (mouseloop without 5′ ISD, and without eSL) (equivalent to
synthetic SR220521); Nucleotide sequences of plasmid elements in order N-terminal to
C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgt |
| | ttatcgaaccgaataaggaactgtgctttgtgattcacatatcagtggaggggtgtggaaa |
| | tggcaccttgatctcaccctcatcgaaagtggagttgatgtcctTccctggctcgctacag |
| | acgcacttccgc (SEQ ID NO: 134) |
| extra nt | aa |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/long) | ggttttctgacttcggtcggaaaacccct (SEQ ID NO: 119) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/long) | ggttttctgacttcggtcggaaaacccct (SEQ ID NO: 119) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05868 Nucleotide Sequence (whole transgene from ITR to ITR):
ctgcgcgctcgctcgctcactgaggccgcccgggcaaagcccgggcgtcgggcgacctttggtcgcccggcctcagtgagcgagc
gagcgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaa
ccgaataaggaactgtgctttgtgattcacatatcagtggaggggtgtggaaatggcaccttgatctcaccctcatcgaaagtgg
agttgatgtcctTccctggctcgctacagacgcacttccgcaagttggagcaagattatcctaaggggtaatacagtacccAATT
TTTGGAgcaggttttctgacttcggtcggaaaacccctcccaatttcactggtctacaatgaaagcaaaacagttctcttccccg
ctccccggtgtgtgagaggggctttgatccttctctggtttcctaggaaacgcgtatgtgTTGTTCCTCTTAGTGTTAATTCACA
CTAAAGACTGTGCATCCGACTCCTACATTTATGAAAGTAAATGCCTGTTGTTAGAACAAAAAAGGCTACAG
AACAAAAAACAAAGCGAAATACCATCTGCTTTAGGTTCAGTGTGGTATTTTCCCGCT
GACAGGGAGGCGGGTTTTTGGGTACAGGAAACGAGTCACTATGGAGGCGGTACTAT
GTAGATGAGAATTCAGGTGCAAACTGGGAAAAGCAACTGCTTCCAAATATTTGTGA
TTTTTACAGTGTAGTTTTGGAAAAACTCTTAGCCTACCAATTCTTCTAAGTGTTTTAA
AATGTGGGAGCCAGTACACATGAAGTTATAGAGTGTTTTAATGAGGCTTAAATATTT
ACCGTAACTATGAAATGCTACGCATATCATGCTGTTCAGGCTCCGTGGCCACGCAAC
TCgttggagcaagattatcctaaggggtaatacagtacccAATTTTTGGAGcaggttttctgacttcggtcggaaaacccctGT
TTACTTGGTTTTAAAAATAGCTTGCACTAGCGATACGGAATATGGTTATTAGGTTTG
TTAGGCATCATGTCGTGTCTTACTATAGAAAAATAACGTAGTGTTCATTTTAGCCTG
CCTGTATGTGTTAATTTGTCCTTATTGCGCATTGTTCTTGTTAAGTCTTCTGTAAGGA
GTTGCGGGTTTCAAACTGTCAGTCTGAGAGCAGAATTCGATATCTAGATCTCGAGGT
AACCACGTGCGGACCCAACGGCCGCaggaacccctagtgatggagttggccactccctctctgcgcgctcgctcgctcactgag
gccgggcgaccaaaggtcgcccgacgcccgggctttgcccgggggcctcagtgagcgagcgagcgcgcagctgcctgc
agg (SEQ ID NO: 174).

Another illustrative AAV vector of the disclosure targeting SCN1A exon 20N is A06047. The elements of A06047 are set forth in Table 19. In some aspects a nucleic acid sequence encoding AAV vector A06047 comprises SEQ ID NO: 175.

TABLE 19
A06047: scAAV-2x_mU7prom-SCN1A1z15/z6-mU7term_mU1prom-SCNIA1z15/z6-
mU1term; (mouseloop without 5′ ISD and without eSL, short SL) (equivalent to synthetic
SR220590); Nucleotide sequences of plasmid elements in order N-terminal to C-term

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/short) | ggttttctgacttcggtoggaaaacc (SEQ ID NO: 120) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/short) | ggttttctgacttcggtcggaaaacc (SEQ ID NO: 120) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A06047 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaaaggggtaatacagtacccccaagttggagcaagattatcctatacAATTTTTGGAGc
aggttttctgacttcggtcggaaaacccccaatttcactggtctacaatgaaagcaaaacagttctcttccccgctccccggtgtgtg
agaggggctttgatccttctctggtttcctaggaaacgcgtatgtgTTGTTCCTCTTAGTGTTAATTCACACTAAAGACTGTG
CATCCGACTCCTACATTTATGAAAGTAAATGCCTGTTGTTAGAACAAAAAAGGCTAC
AGAACAAAAAACAAAGCGAAATACCATCTGCTTTAGGTTCAGTGTGGTATTTTCCCG
CTGACAGGGAGGCGGGTTTTTGGGTACAGGAAACGAGTCACTATGGAGGCGGTACT
ATGTAGATGAGAATTCAGGTGCAAACTGGGAAAAGCAACTGCTTCCAAATATTTGT
GATTTTTACAGTGTAGTTTTGGAAAAACTCTTAGCCTACCAATTCTTCTAAGTGTTTT
AAAATGTGGGAGCCAGTACACATGAAGTTATAGAGTGTTTTAATGAGGCTTAAATA
TTTACCGTAACTATGAAATGCTACGCATATCATGCTGTTCAGGCTCCGTGGCCACGC
AACTCaggggtaatacagtacccccaagttggagcaagattatcctatacAATTTTTGGAGcaggttttctgacttcggtcgga
aaaccGTTTACTTGGTTTTAAAAATAGCTTGCACTAGCGATACGGAATATGGTTATTAG
GTTTGTTAGGCATCATGTCGTGTCTTACTATAGAAAAATAACGTAGTGTTCATTTTAG
CCTGCCTGTATGTGTTAATTTGTCCTTATTGCGCATTGTTCTTGTTAAGTCTTCTGTAA
GGAGTTGCGGGTTTCAAACTGTCAGTCTGAGAGCAGAATTCGATATCTAGATCTCGA
GGTAACCACGTGCGGACCCAACGGCCGCaggaacccctagtgatggagttggccactccctctctgcgcgctcg
ctcgctcactgaggccgggcgaccaaaggtcgcccgacgcccgggctttgcccgggcggcctcagtgagcgagcgagcgcgcagctg
cctgcagg (SEQ ID NO: 175).

Another illustrative AAV vector of the disclosure targeting SCN1A exon 20N is A06048. The elements of A06048 are set forth in Table 20. In some aspects a nucleic acid sequence encoding AAV vector A06048 comprises SEQ ID NO: 176.

TABLE 20
A06048: scAAV-2x_mU7prom-SCNIA1z11/z15-mU7term_mU1prom-
SCNIA1z11/z15-mU1term; (mouseloop without 5′ ISD, and without eSL, short SL) (equivalent
to synthetic SR220591); Nucleotide sequences of plasmid elements in order N-terminal to
C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/short) | ggttttctgacttcggtcggaaaacc (SEQ ID NO: 120) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/short) | ggttttctgacttcggtcggaaaacc (SEQ ID NO: 120) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggggcctcagtgag |
| | cgagcgagcgcgcagctgcctgcagg (SEQ ID NO: 159) |

A06048 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaagttggagcaagattatcctaaggggtaatacagtacccAATTTTTGGAgcaggtttt
ctgacttcggtcggaaaacccccaatttcactggtctacaatgaaagcaaaacagttctcttccccgctccccggtgtgtgagagggg
ctttgatccttctctggtttcctaggaaacgcgtatgtgTTGTTCCTCTTAGTGTTAATTCACACTAAAGACTGTGCATCC
GACTCCTACATTTATGAAAGTAAATGCCTGTTGTTAGAACAAAAAAGGCTACAGAA
CAAAAAACAAAGCGAAATACCATCTGCTTTAGGTTCAGTGTGGTATTTTCCCGCTGA
CAGGGAGGCGGGTTTTTGGGTACAGGAAACGAGTCACTATGGAGGCGGTACTATGT
AGATGAGAATTCAGGTGCAAACTGGGAAAAGCAACTGCTTCCAAATATTTGTGATTT
TTACAGTGTAGTTTTGGAAAAACTCTTAGCCTACCAATTCTTCTAAGTGTTTTAAAAT
GTGGGAGCCAGTACACATGAAGTTATAGAGTGTTTTAATGAGGCTTAAATATTTACC
GTAACTATGAAATGCTACGCATATCATGCTGTTCAGGCTCCGTGGCCACGCAACTCgt
tggagcaagattatcctaaggggtaatacagtacccAATTTTTGGAGcaggttttctgacttcggtcggaaaaccGTTTACT
TGGTTTTAAAAATAGCTTGCACTAGCGATACGGAATATGGTTATTAGGTTTGTTAGG
CATCATGTCGTGTCTTACTATAGAAAAATAACGTAGTGTTCATTTTAGCCTGCCTGTA
TGTGTTAATTTGTCCTTATTGCGCATTGTTCTTGTTAAGTCTTCTGTAAGGAGTTGCG
GGTTTCAAACTGTCAGTCTGAGAGCAGAATTCGATATCTAGATCTCGAGGTAACCAC
GTGCGGACCCAACGGCCGCaggaacccctagtgatggagttggccactccctctctgcgcgctcgctcgctcactgagg
ccgggcgaccaaaggtcgcccgacgcccgggctttgcccgggggcctcagtgagcgagcgagcgcgcagctgcctgcagg (SEQ
ID NO: 176).

Another illustrative AAV vector of the disclosure targeting SCN1A exon 20N is A05869. The elements of A05869 are set forth in Table 21. In some aspects a nucleic acid sequence encoding AAV vector A05869 comprises SEQ ID NO: 177.

TABLE 21
A05869: scAAV-2x_mU7prom-SCN1A1z15/z6-mU7term_mU1prom-SCNIA1z15/z6-
mU1term; (sheeploop without 5′ ISD and without eSL, long) (equivalent to synthetic SR220523);
Nucleotide sequences of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/ long) | GGCTTTCCGGCCTCCGCCGGAAAGCCCCT (SEQ ID NO: 123) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/ long) | GGCTTTCCGGCCTCCGCCGGAAAGCCCCT (SEQ ID NO: 123) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05869 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaaaggggtaatacagtacccccaagttggagcaagattatcctatacAATTTTTGGAGc
aGGCTTTCCGGCCTCCGCCGGAAAGCCCCTcccaatttcactggtctacaatgaaagcaaaacagttctcttccccgctc
cccggtgtgtgagaggggctttgatccttctctggtttcctaggaaacgcgtatgtgTTGTTCCTCTTAGTGTTAATTCA
CACTAAAGACTGTGCATCCGACTCCTACATTTATGAAAGTAAATGCCTGTTGTTAGA
ACAAAAAAGGCTACAGAACAAAAAACAAAGCGAAATACCATCTGCTTTAGGTTCAG
TGTGGTATTTTCCCGCTGACAGGGAGGCGGGTTTTTGGGTACAGGAAACGAGTCACT
ATGGAGGCGGTACTATGTAGATGAGAATTCAGGTGCAAACTGGGAAAAGCAACTGC
TTCCAAATATTTGTGATTTTTACAGTGTAGTTTTGGAAAAACTCTTAGCCTACCAATT
CTTCTAAGTGTTTTAAAATGTGGGAGCCAGTACACATGAAGTTATAGAGTGTTTTAA
TGAGGCTTAAATATTTACCGTAACTATGAAATGCTACGCATATCATGCTGTTCAGGC
TCCGTGGCCACGCAACTCaggggtaatacagtacccccaagttggagcaagattatcctatacAATTTTTGGAGc
aGGCTTTCCGGCCTCCGCCGGAAAGCCCCTGTTTACTTGGTTTTAAAAATAGCTTGCA
CTAGCGATACGGAATATGGTTATTAGGTTTGTTAGGCATCATGTCGTGTCTTACTAT
AGAAAAATAACGTAGTGTTCATTTTAGCCTGCCTGTATGTGTTAATTTGTCCTTATTG
CGCATTGTTCTTGTTAAGTCTTCTGTAAGGAGTTGCGGGTTTCAAACTGTCAGTCTGA
GAGCAGAATTCGATATCTAGATCTCGAGGTAACCACGTGCGGACCCAACGGCCGCag
gaacccctagtgatggagttggccactccctctctgcgcgctcgctcgctcactgaggccgggcgaccaaaggtcgcccgacgcccggg
ctttgcccgggcggcctcagtgagcgagcgagcgcgcagctgcctgcagg (SEQ ID NO: 177).

Another illustrative AAV vector of the disclosure targeting SCN1A exon 20N is A05870. The elements of A05870 are set forth in Table 22. In some aspects a nucleic acid sequence encoding AAV vector A05870 comprises SEQ ID NO: 178.

TABLE 22
A05870: scAAV-2x_mU7prom-SCN1A1z11/215-mU7term_mU1prom-
SCN1A1z11/z15-mU1term; (sheeploop without 5′ ISD, and without eSL, long) (equivalent to
synthetic SR220524); Nucleotide sequences of plasmid elements in order N-terminal to C-
terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/long) | GGCTTTCCGGCCTCCGCCGGAAAGCCCCT (SEQ ID |
| | NO: 123) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/long) | GGCTTTCCGGCCTCCGCCGGAAAGCCCCT (SEQ ID |
| | NO: 123) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A05870 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaagttggagcaagattatcctaaggggtaatacagtacccAATTTTTGGAGcaGGCTTT
CCGGCCTCCGCCGGAAAGCCCCTcccaatttcactggtctacaatgaaagcaaaacagttctcttccccgctccccggt
gtgtgagaggggctttgatccttctctggtttcctaggaaacgcgtatgtgTTGTTCCTCTTAGTGTTAATTCACACT
AAAGACTGTGCATCCGACTCCTACATTTATGAAAGTAAATGCCTGTTGTTAGAACAA
AAAAGGCTACAGAACAAAAAACAAAGCGAAATACCATCTGCTTTAGGTTCAGTGTG
GTATTTTCCCGCTGACAGGGAGGCGGGTTTTTGGGTACAGGAAACGAGTCACTATGG
AGGCGGTACTATGTAGATGAGAATTCAGGTGCAAACTGGGAAAAGCAACTGCTTCC
AAATATTTGTGATTTTTACAGTGTAGTTTTGGAAAAACTCTTAGCCTACCAATTCTTC
TAAGTGTTTTAAAATGTGGGAGCCAGTACACATGAAGTTATAGAGTGTTTTAATGAG
GCTTAAATATTTACCGTAACTATGAAATGCTACGCATATCATGCTGTTCAGGCTCCG
TGGCCACGCAACTCgttggagcaagattatcctaaggggtaatacagtacccAATTTTTGGAGcaGGCTTTC
CGGCCTCCGCCGGAAAGCCCCTGTTTACTTGGTTTTAAAAATAGCTTGCACTAGCGA
TACGGAATATGGTTATTAGGTTTGTTAGGCATCATGTCGTGTCTTACTATAGAAAAA
TAACGTAGTGTTCATTTTAGCCTGCCTGTATGTGTTAATTTGTCCTTATTGCGCATTG
TTCTTGTTAAGTCTTCTGTAAGGAGTTGCGGGTTTCAAACTGTCAGTCTGAGAGCAG
AATTCGATATCTAGATCTCGAGGTAACCACGTGCGGACCCAACGGCCGCaggaacccctag
tgatggagttggccactccctctctgcgcgctcgctcgctcactgaggccgggcgaccaaaggtcgcccgacgcccgggctttgcccgg
gcggcctcagtgagcgagcgagcgcgcagctgcctgcagg (SEQ ID NO: 178).

Another illustrative AAV vector of the disclosure targeting SCN1A exon 20N is A06049. The elements of A06049 are set forth in Table 23. In some aspects a nucleic acid sequence encoding AAV vector A06049 comprises SEQ ID NO: 179.

TABLE 23
A06049: scAAV-2x_mU7prom-SCN1A1z15/z6-mU7term_mU1prom-SCNIA1z15/z6-
mU1term; (sheeploop without 5′ ISD and without eSL, short) (equivalent to synthetic
SR220592); Nucleotide sequences of plasmid elements in order N-terminal to C-terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/short) | GGCTTTCCGGCCTCCGCCGGAAAGCC (SEQ ID NO: 124) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z15/z6 | aggggtaatacagtacccccaagttggagcaagattatcctatac (SEQ ID NO: 55) |
| SMOPT | AATTTTTGGAGca (SEQ ID NO: 160) |
| Stem loop (native/ long) | GGCTTTCCGGCCTCCGCCGGAAAGCC (SEQ ID NO: 124) |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3?ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A06049 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaaaggggtaatacagtacccccaagttggagcaagattatcctatacAATTTTTGGAGc
aGGCTTTCCGGCCTCCGCCGGAAAGCCcccaatttcactggtctacaatgaaagcaaaacagttctcttccccgctccccg
gtgtgtgagaggggctttgatccttctctggtttcctaggaaacgcgtatgtgTTGTTCCTCTTAGTGTTAATTCACAC
TAAAGACTGTGCATCCGACTCCTACATTTATGAAAGTAAATGCCTGTTGTTAGAACA
AAAAAGGCTACAGAACAAAAAACAAAGCGAAATACCATCTGCTTTAGGTTCAGTGT
GGTATTTTCCCGCTGACAGGGAGGCGGGTTTTTGGGTACAGGAAACGAGTCACTATG
GAGGCGGTACTATGTAGATGAGAATTCAGGTGCAAACTGGGAAAAGCAACTGCTTC
CAAATATTTGTGATTTTTACAGTGTAGTTTTGGAAAAACTCTTAGCCTACCAATTCTT
CTAAGTGTTTTAAAATGTGGGAGCCAGTACACATGAAGTTATAGAGTGTTTTAATGA
GGCTTAAATATTTACCGTAACTATGAAATGCTACGCATATCATGCTGTTCAGGCTCC
GTGGCCACGCAACTCaggggtaatacagtacccccaagttggagcaagattatcctatacAATTTTTGGAGcaGG
CTTTCCGGCCTCCGCCGGAAAGCCGTTTACTTGGTTTTAAAAATAGCTTGCACTAGC
GATACGGAATATGGTTATTAGGTTTGTTAGGCATCATGTCGTGTCTTACTATAGAAA
AATAACGTAGTGTTCATTTTAGCCTGCCTGTATGTGTTAATTTGTCCTTATTGCGCAT
TGTTCTTGTTAAGTCTTCTGTAAGGAGTTGCGGGTTTCAAACTGTCAGTCTGAGAGC
AGAATTCGATATCTAGATCTCGAGGTAACCACGTGCGGACCCAACGGCCGCaggaaccc
ctagtgatggagttggccactccctctctgcgcgctcgctcgctcactgaggccgggcgaccaaaggtcgcccgacgcccgggctttgcc
cgggcggcctcagtgagcgagcgagcgcgcagctgcctgcagg (SEQ ID NO: 179).

Another illustrative AAV vector of the disclosure targeting SCN1A exon 20N is A06050. The elements of A06050 are set forth in Table 24. In some aspects a nucleic acid sequence encoding AAV vector A06050 comprises SEQ ID NO: 180.

TABLE 24
A06050: scAAV-2x_mU7prom-SCNIA1z11/z15-mU7term_mU1prom-
SCN1A1z11/z15-mU1term; (sheeploop without 5′ ISD, and without SL, short) (equivalent to
synthetic SR220593); Nucleotide sequences of plasmid elements in order N-terminal to C-
terminal

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | (SEQ ID NO: 158) |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc (SEQ ID NO: 134) |
| extra nt | aa |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/short) | GGCTTTCCGGCCTCCGCCGGAAAGCC (SEQ ID NO: 124) |
| Mouse U7 terminator | cccaatttcactggtctacaatgaaagcaaaacagttctcttccccgctccccggtgtgtg |
| | agaggggctttgatccttctctggtttcctaggaaacgcgtatgtg (SEQ ID NO: |
| | 146) |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C (SEQ ID NO: 132) |
| Fusion spacer z11/z15 | gttggagcaagattatcctaaggggtaatacagtaccc (SEQ ID NO: 57) |
| SMOPT | AATTTTTGGAGca (SEQ ID NO: 160) |
| Stem loop (native/short) | GGCTTTCCGGCCTCCGCCGGAAAGCC (SEQ ID NO: 124) |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA (SEQ ID NO: 144) |
| 3′ITR | aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg (SEQ ID NO: 159) |

A06050 Nucleotide Sequence (whole transgene from ITR to ITR):
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcactgActc
atttgcatagcctttacaagcggtcacaaactcaagaaacgagcggttttaatagtcttttagaatattgtttatcgaaccgaataag
gaactgtgctttgtgattcacatatcagtggaggggtgtggaaatggcaccttgatctcaccctcatcgaaagtggagttgatgtcct
TccctggctcgctacagacgcacttccgcaagttggagcaagattatcctaaggggtaatacagtacccAATTTTTGGAGcaGGCTTT
CCGGCCTCCGCCGGAAAGCCcccaatttcactggtctacaatgaaagcaaaacagttctcttccccgctccccggtgtgtg
agaggggctttgatccttctctggtttcctaggaaacgcgtatgtgTTGTTCCTCTTAGTGTTAATTCACACTAAA
GACTGTGCATCCGACTCCTACATTTATGAAAGTAAATGCCTGTTGTTAGAACAAAAA
AGGCTACAGAACAAAAAACAAAGCGAAATACCATCTGCTTTAGGTTCAGTGTGGTA
TTTTCCCGCTGACAGGGAGGCGGGTTTTTGGGTACAGGAAACGAGTCACTATGGAG
GCGGTACTATGTAGATGAGAATTCAGGTGCAAACTGGGAAAAGCAACTGCTTCCAA
ATATTTGTGATTTTTACAGTGTAGTTTTGGAAAAACTCTTAGCCTACCAATTCTTCTA
AGTGTTTTAAAATGTGGGAGCCAGTACACATGAAGTTATAGAGTGTTTTAATGAGGC
TTAAATATTTACCGTAACTATGAAATGCTACGCATATCATGCTGTTCAGGCTCCGTG
GCCACGCAACTCgttggagcaagattatcctaaggggtaatacagtacccAATTTTTGGAGcaGGCTTTCCG
GCCTCCGCCGGAAAGCCGTTTACTTGGTTTTAAAAATAGCTTGCACTAGCGATACGG
AATATGGTTATTAGGTTTGTTAGGCATCATGTCGTGTCTTACTATAGAAAAATAACG
TAGTGTTCATTTTAGCCTGCCTGTATGTGTTAATTTGTCCTTATTGCGCATTGTTCTTG
TTAAGTCTTCTGTAAGGAGTTGCGGGTTTCAAACTGTCAGTCTGAGAGCAGAATTCG
ATATCTAGATCTCGAGGTAACCACGTGCGGACCCAACGGCCGCaggaacccctagtgatggagtt
ggccactccctctctgcgcgctcgctcgctcactgaggccgggcgaccaaaggtcgcccgacgcccgggctttgcccgggggcctca
gtgagcgagcgagcgcgcagctgcctgcagg (SEQ ID NO: 180).

Provided herein is a vector construct targeting SCN1A comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 162-180. In some embodiments, the vector construct targeting SCN1A comprises the nucleic acid sequence set forth in SEQ ID NO: 162. In some embodiments, the vector construct targeting SCN1A comprises the nucleic acid sequence set forth in SEQ ID NO: 163. In some embodiments, the vector construct targeting SCN1A comprises the nucleic acid sequence set forth in SEQ ID NO: 164. In some embodiments, the vector construct targeting SCN1A comprises the nucleic acid sequence set forth in SEQ ID NO: 165. In some embodiments, the vector construct targeting SCN1A comprises the nucleic acid sequence set forth in SEQ ID NO: 166. In some embodiments, the vector construct targeting SCN1A comprises the nucleic acid sequence set forth in SEQ ID NO: 167. In some embodiments, the vector construct targeting SCN1A comprises the nucleic acid sequence set forth in SEQ ID NO: 168. In some embodiments, the vector construct targeting SCN1A comprises the nucleic acid sequence set forth in SEQ ID NO: 169. In some embodiments, the vector construct targeting SCN1A comprises the nucleic acid sequence set forth in SEQ ID NO: 170. In some embodiments, the vector construct targeting SCN1A comprises the nucleic acid sequence set forth in SEQ ID NO: 171. In some embodiments, the vector construct targeting SCN1A comprises the nucleic acid sequence set forth in SEQ ID NO: 172. In some embodiments, the vector construct targeting SCN1A comprises the nucleic acid sequence set forth in SEQ ID NO: 173. In some embodiments, the vector construct targeting SCN1A comprises the nucleic acid sequence set forth in SEQ ID NO: 174. In some embodiments, the vector construct targeting SCN1A comprises the nucleic acid sequence set forth in SEQ ID NO: 175. In some embodiments, the vector construct targeting SCN1A comprises the nucleic acid sequence set forth in SEQ ID NO: 176. In some embodiments, the vector construct targeting SCN1A comprises the nucleic acid sequence set forth in SEQ ID NO: 177. In some embodiments, the vector construct targeting SCN1A comprises the nucleic acid sequence set forth in SEQ ID NO: 178. In some embodiments, the vector construct targeting SCN1A comprises the nucleic acid sequence set forth in SEQ ID NO: 179. In some embodiments, the vector construct targeting SCN1A comprises the nucleic acid sequence set forth in SEQ ID NO: 180.

### Nucleic Acids

An NOI (nucleotide sequence of interest) includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region but need not be a coding region. An NOI can be RNA/DNA in a sense or antisense orientation. An NOI can be an snRNA. NOIs are also referred herein, without limitation, as transgenes, heterologous sequences, genes, therapeutic genes. An NOI may also encode an RNA (ribonucleoprotein complex) a POI (protein of interest), a partial POI, a mutated version or variant of a POI. A POI may be analogous to or correspond to a wild-type protein. A POI may also be a fusion protein or ribonucleoprotein complex such as an snRNP. In some aspects RNA sequences disclosed herein may be represented as DNA sequences and it is within the ability of the skilled artisan to derive the sequence of an RNA sequence from a DNA sequence. For example, spacer sequences of the disclosure can represent uracil bases as either a U or T. The skilled artisan would readily understand that an RNA sequence can interchangeably use a T or U to indicate a uracil.

### Codon Optimization

In some embodiments, NOIs or transgenes such as nucleic acid sequences of the disclosure are codon optimized nucleic acid sequences.

Codon-optimization is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences can be generated. In some embodiments, such a sequence is optimized for expression in a host or target cell, such as a host cell used to express the snRNA or a cell in which the disclosed methods are practiced (such as in a mammalian cell, e.g., a human cell). Codon preferences and codon usage tables for a particular species can be used to engineer isolated nucleic acid molecules encoding an snRNA that takes advantage of the codon usage preferences of that particular species. In some embodiments, an isolated nucleic acid molecule (which can be part of a vector) includes at least one coding sequence that is codon optimized for expression in a eukaryotic cell, or at least one coding sequence codon optimized for expression in a human cell. In one embodiment, such a codon optimized coding sequence has at least 80%, at least 85%, at least 90%, at least 92%, at least 95%, at least 96%, at least 97%, at least 98%, at least 99%, or 100% sequence identity to its corresponding wildtype or originating sequence. In another embodiment, a eukaryotic cell codon optimized nucleic acid sequence encodes snRNA having at least 85%, at least 90%, at least 92%, at least 95%, at least 96%, at least 97%, at least 98%, at least 99%, or 100% sequence identity to its corresponding wildtype or originating sequence. In another embodiment, a variety of clones containing functionally equivalent nucleic acids may be routinely generated, such as nucleic acids which differ in sequence but which encode the same sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

In some embodiments, the codon optimized sequence exhibits at least 5%, at least 10%, at least 20%, at least 30%, at least 50%, at least 75%, at least 100%, at least 200%, at least 300%, at least 500%, or at least 1000% increased transcription or translation in a human subject relative to a wild-type or non-codon optimized nucleic acid sequence.

In some aspects a codon optimized nucleic acid sequence exhibits increased stability. In some aspects a codon optimized nucleic acid sequence exhibits increased stability through increased resistance to hydrolysis. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects a codon optimized nucleic acid sequence can comprise no donor splice sites. In some aspects a codon optimized nucleic acid sequence can comprise no more than about one, or about two, or about three, or about four, or about five, or about six, or about seven, or about eight, or about nine, or about ten donor splice sites. In some aspects a codon optimized nucleic acid sequence comprises at least one, or at least two, or at least three, or at least four, or at least five, or at least six, or at least seven, or at least eight, or at least nine, or at least ten fewer donor splice sites as compared to a non-codon optimized nucleic acid sequence.

Without wishing to be bound by theory, the removal of donor splice sites in the codon optimized nucleic acid sequence can unexpectedly and unpredictably increase expression of protein of interest in vivo, as cryptic splicing is prevented. Moreover, cryptic splicing may vary between different subjects, meaning that the expression level of a protein comprising donor splice sites may unpredictably vary between different subjects. Such unpredictability is unacceptable in the context of human therapy. Accordingly, the codon optimized nucleic acid sequences which lacks donor splice sites, unexpectedly and surprisingly allows for increased expression of the protein in human subjects and regularizes expression of the protein across different human subjects.

In some aspects a codon optimized nucleic acid sequence can have a GC content that differs from the GC content of the non-codon optimized nucleic acid sequence. In some aspects the GC content of a codon optimized nucleic acid sequence is more evenly distributed across the entire nucleic acid sequence, as compared to the non-codon optimized nucleic acid sequence.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects a codon optimized nucleic acid sequence can have fewer repressive microRNA target binding sites as compared to the non-codon optimized nucleic acid sequence. In some aspects, a codon optimized nucleic acid sequence can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence.

Without wishing to be bound by theory, by having fewer repressive microRNA target binding sites, the codon optimized nucleic acid sequence unexpectedly exhibits increased expression in a human subject.

It should be understood, although not always explicitly stated that the sequences provided herein can be used to provide the expression product as well as substantially identical sequences that encode an RNA or express and produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

Also provided herein are cells comprising the RNA targeting systems and expression constructs described herein. In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell. In one embodiment, a cell or cells of a patient treated with compositions disclosed herein include, without limitation, skeletal muscle (developing and mature muscle fibers and satellite cells), neuromuscular junction, cardiomyocytes, smooth muscle cells, peripheral nervous system (neurons), peripheral motor neurons, and/or sensory neurons.

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, sensory neuron, cortical or GABAergic inhibitory interneurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the disclosure, a somatic cell is an ocular cell. An ocular cell includes, without limitation, corneal epithelial cells, keratyocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells. In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Muller glial cell, a rod cell, or a cone cell. In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of encoding an RNA or expressing an NOI in a cell using the snRNA systems disclosed herein. In one embodiment, the disclosure provides a method of modifying an RNA or the activity of a protein encoded by an RNA molecule comprising contacting the composition of the disclosure and the target RNA molecule under conditions suitable for binding to the RNA molecule.

The disclosure provides a method of modifying the level of expression of an RNA molecule of the disclosure of a protein encoded by the RNA molecule comprising contacting the composition of the disclosure and a cell comprising the RNA molecule under conditions suitable for binding to the RNA molecule. In some embodiments, the cell is in vivo, in vitro, ex vivo or in situ. In some embodiments, the composition of the disclosure comprises a vector comprising snRNA sequences. In some embodiments, the vector is an AAV.

The disclosure provides a method of modifying the level of expression of an RNA molecule of the disclosure or a protein encoded by the RNA molecule comprising contacting a composition of the disclosure and the RNA molecule under conditions suitable for knocking down, blocking, splicing, multi-targeting, restore frame, or editing the target RNA. In some embodiments, the composition of the disclosure comprises a vector comprising snRNA sequences. In some embodiments, the vector is an AAV.

The disclosure provides a method of modifying a target RNA or an activity of a protein encoded by a target RNA molecule comprising contacting a composition and a cell comprising the RNA molecule under conditions suitable knocking down, blocking, splicing, multi-targeting, restore frame, or editing the target RNA. In some embodiments, the cell is in vivo, in vitro, ex vivo or in situ. In some embodiments, the composition comprises a vector comprising the snRNA sequences disclosed herein. In some embodiments, the vector is an AAV.

The disclosure provides a method of treating a disease or disorder comprising administering to a subject a therapeutically effective amount of an snRNA composition of the disclosure.

The disclosure provides a method of treating a disease in a patient in need of such treatment comprising administering to the patient a therapeutically effective amount of an snRNA composition of the disclosure, wherein the composition comprises a vector comprising snRNA sequences disclosed herein, wherein the composition restores frame and function and increases a level of expression of a targeted RNA (compared to the level of expression of a targeted RNA treated with a non-targeting (NT) control or compared to no treatment). In another embodiment, the level of increase is 1-fold or greater. In another embodiment, the level of increase is 2-fold, 3-fold, 4-fold, 5-fold, 6-fold, 7-fold, 8-fold, 9-fold or 10-fold. In another embodiment, the level of increase is 10-fold or greater. In another embodiment, the level of increase is between 10-fold and 20-fold. In another embodiment, the level of increase is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% restoration and/or function. In one embodiment, the % restoration and/or function is any of 20-99%, 25%-99%, 50%-99%, 80%-99%, 90%-99%, 95%-99%. In one embodiment, the % restoration and/or function is 90%, 91%, 92%, 93%, 94%, 95%, 96%, 97%, 98%, or 99%. In another embodiment the gene therapy, sequences disclosed herein, promotes an increase level of expression of the correct RNA transcript, improving protein expression and function. In another embodiment, % up-regulation is 1.5-fold or higher of the targeted RNA. In some embodiments, the targeted RNA is SCN1A. In some embodiments, the targeted RNA is SCN1A Exon 20N. In some embodiments, the targeted RNA is SYNGAP1. In some embodiments, the targeted RNA is SYNGAP1 Exon 11 or poison Exon 11.

The disclosure further provides a method of treating a disease or disorder in a subject comprising administering an RNA-targeting nucleic acid molecule (i.e. an snRNA of the disclosure) or an AAV vector comprising an snRNA of the disclosure.

In some aspects, the disease or disorder is an epilepsy. In some aspects, the epilepsy is a genetic epilepsy.

In some aspects the disease or disorder is Dravet Syndrome. In some aspects the RNA-targeting nucleic acid molecule or AAV vector targets an RNA sequence encoding NaV1.1. (SCN1A). In some aspects the RNA sequence encoding SCN1A comprises an intronic or exonic sequence. In some aspects the exonic sequence comprises exon 20N or a flanking region thereof of SCN1A.

In some aspects the disease or disorder is SYNGAP1-Related Epilepsy. In some aspects the RNA-targeting nucleic acid molecule or AAV vector targets an RNA sequence encoding Synaptic Ras GTPase-activating protein 1 (SYNGAP1). In some aspects the RNA sequence encoding SYNGAP1 comprises an intronic or exonic sequence. In some aspects the exonic sequence comprises exon 11 or poison exon 11 or a flanking region thereof of SYNGAP1.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure a subject of the disclosure is a mammal. In some embodiments, a subject of the disclosure is a non-human mammal.

In some embodiments of the methods of the disclosure a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder. Signs and symptoms of Dravet Syndrome that may be reduced in severity or eliminated using the compositions described herein include seizures (e.g., focal seizures, generalized seizures, febrile seizures or grand mal seizures), ataxia, low motor tone, and crouched gait. In some embodiments, the seizures are not well controlled using standard of care seizure medications (e.g., valproic acid, clobazam, topiramate, stiripentol, fenfluramine, and/or cannabidiol).

In some embodiments of the methods of the disclosure a therapeutically effective amount eliminates the disease or disorder (e.g., Dravet Syndrome).

In some embodiments of the methods of the disclosure a therapeutically effective amount eliminates the disease or disorder (e.g., SYNGAP1-related epilepsy).

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount of the compositions described herein delays the onset of SYNGAP1-related epilepsy or Dravet Syndrome until the 2^{nd}, 3^{rd}, 4^{th}, 5^{th}, 10^{th}, 15^{th}, 20^{th} or 25^{th} year of life.

In some embodiments, a therapeutically effective amount improves a prognosis for the subject.

In some embodiments of the methods of the disclosure, a composition of the disclosure is administered to the subject via intracerebral administration. In some embodiments of the methods of the disclosure, a composition of the disclosure is administered to the subject by intracerebroventricular injection. In some embodiments, the composition of the disclosure is administered to the subject by an intrastriatal route. In some embodiments, the composition of the disclosure is administered to the subject by a stereotaxic injection or an infusion. In some embodiments, the composition is administered intra-venously. In some embodiments, the composition is administered to the brain. In some embodiments of the methods of the disclosure a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### EXAMPLES

The examples described herein are intended for illustration only and are not intended to limit the inventions claimed.

### Example 1: SCN1A Poison Exon 20N Exclusion

### Material and Methods

### In Vitro Assays

Poison Exon Skipping and Quantification of Productive SCN1A mRNA

Splicing assay to evaluate exon skipping was performed in HeLa or HepG2 cells, transfected with plasmids expressing snRNAs or synthetic snRNAs. For SNC1A+/−GABAergic neurons cells were transduced for 7 days with different MOIs of AAV9-2×snRNA cassettes (A05290 or A05291). RNA was extracted by KingFisher using the Flex MirVana RNA extraction kit or the Zymo's Direct-zol-96 MagBead RNA extraction kit. For exon skipping cDNA was made using SuperScript IV RT. PCR was performed using KOD Xtreme Hot Start polymerase and primers annealing to exon 20 and 21. 1 uL PCR product was analyzed on D1000 TapeStation. For productive SCN1A qRT-PCR was performed with Ultraplex 1-Step Toughmix (QuantaBio) and primer and probes targeting SCN1A exon-exon junction at exon 20/21. Custom GAPDH or ATP5b primers and probes were used to quantify the reference genes for normalization. Relative SCN1A mRNA levels were calculated with the delta-delta Ct method.

For in vivo WT mice were injected ICV at P0 with 5E10 vg/animal of AAV9 A05290 or A05291. At P20 multiple areas of cortex and hippocampus were collected and analyzed for snRNA expression, SCN1A exon 20N skipping, and productive SNC1A RNA and protein expression.

### Results

In Vitro Evaluation of U7 snRNAs Targeting SCN1A Exon 20N

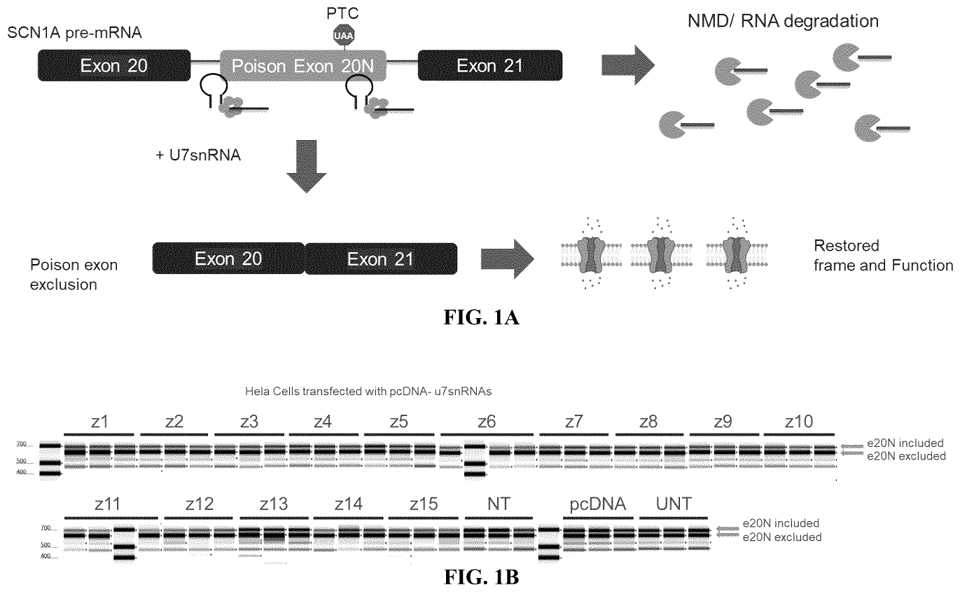

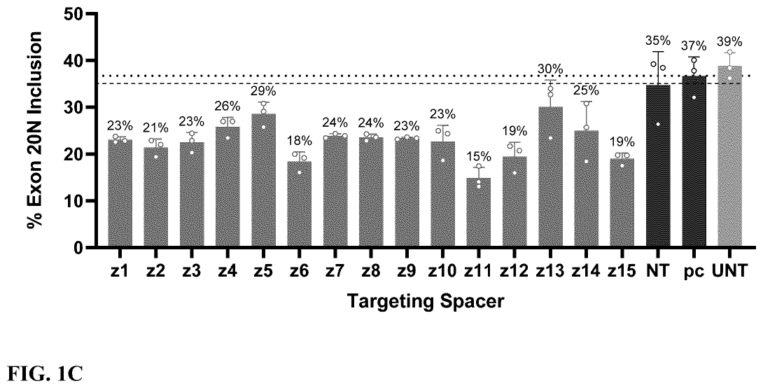

U7 snRNAs were engineered to bind splicing regulatory sequences within the SCN1A poison exon 20N (acceptor and donor sites or splicing enhancer sequences) to promote skipping and restoration of frame and function (FIG. 1A).

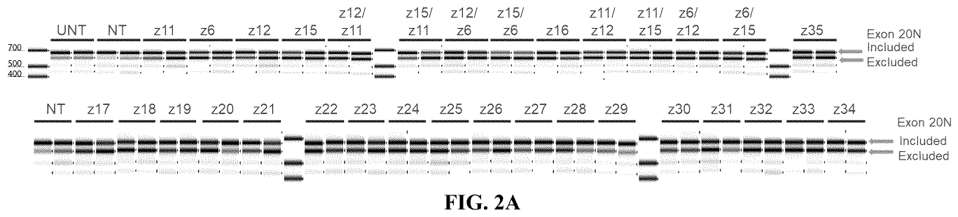

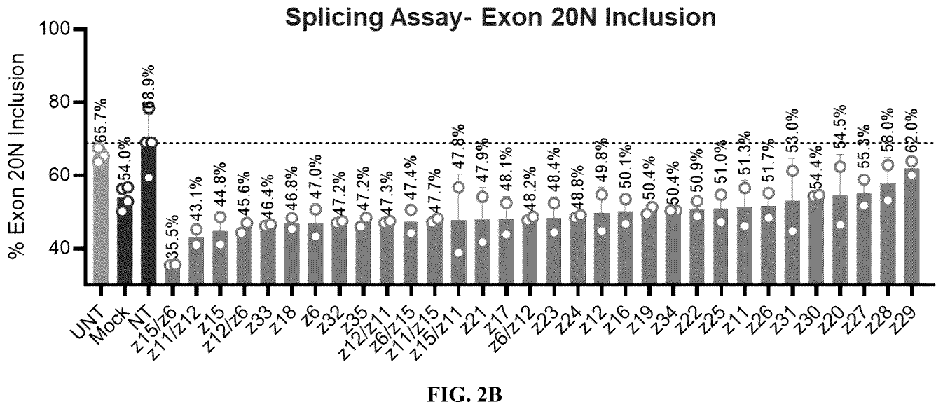

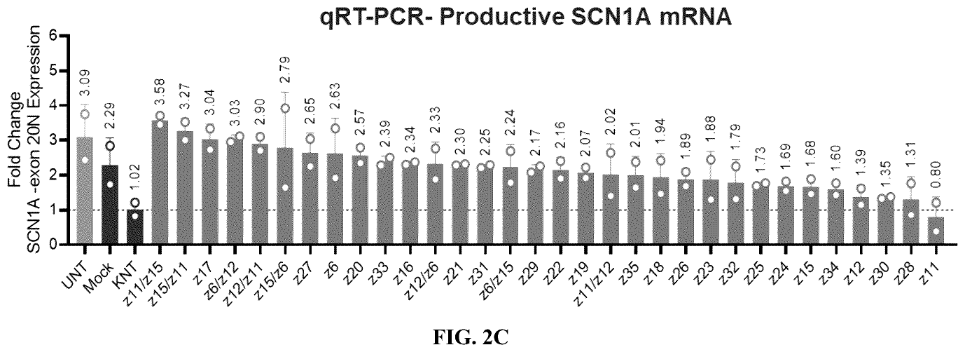

FIG. 1B shows the SCN1A exon 20N non-skipped band (containing the poison exon, top fragment at 669 bp), or the skipped product (exclusion of the poison exon, bottom band at 605 bp) in Hela Cells. Results are quantified in FIG. 1C. FIG. 2A shows a tapestation image of the RT-PCR products after U7 snRNA treatments using single and fusion U7 spacers (cloned into pcDNA plasmid), showing the SCN1A exon 20N non-skipped band, or the skipped product in HepG2 cells. Data are quantified in FIG. 2B. The levels of expression of productive SCN1A (without the poison exon 20N) determined by qRT-PCR following treatment with single and fusion U7 snRNAs is shown in FIG. 2C.

Conclusions: These data show that multiple U7 snRNA spacers (single and fusions) promote poison exon 20N skipping and increase productive SCN1A mRNA expression in vitro. Fusion spacers show more robust skipping in vitro.

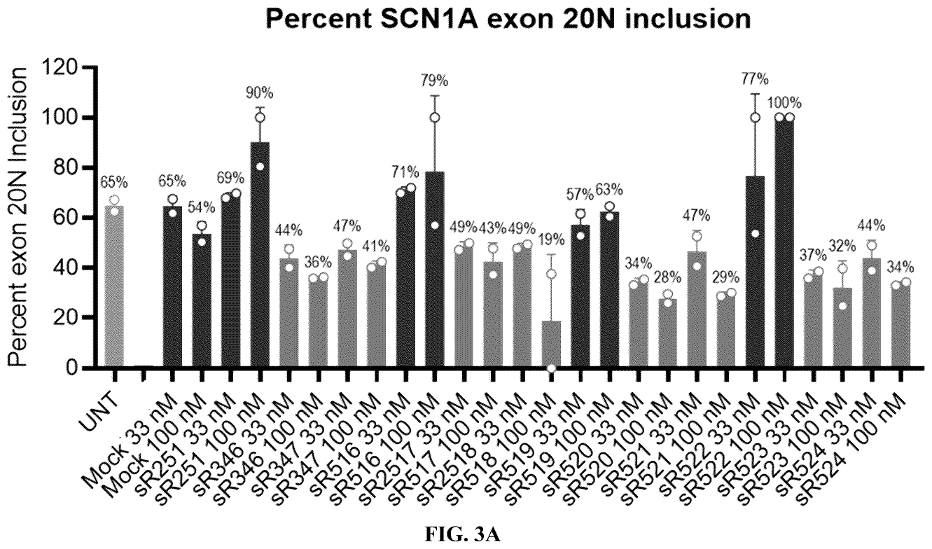

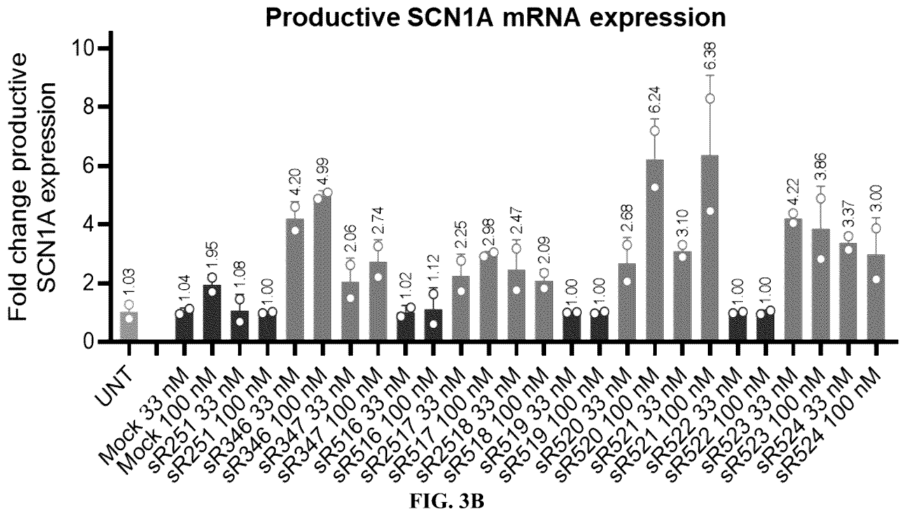

FIG. 3A shows a quantification of the RT-PCR products for percentage of poison exon 20N inclusion after U7 snRNA treatments using fusion synthetic U7 spacers of z15/z6 and z11/z15 with additional modifications to the ISD and stem loop in HepG2 cells. Similarly, FIG. 3B depicts the expression of productive SCN1A (without the poison exon 20N) determined by qRT-PCR following treatment with U7 snRNAs in HepG2 cells transfected with synthetic-U7snRNAs. The snRNA synthetic RNA tested are as follows: SR220251: non targeting; SR220516: non targeting mod1; SR220519: non targeting mod2; SR220522: non targeting mod3; SR220346: z15/z6; SR220517: z15/z6 mod1; SR220520: z15/z6 mod2; SR220523: z15/z6 mod3; SR220347: z11/z15; SR220518: z11/z15 mod1; SR220521: z11/z15 mod2; and SR220524: z11/z15 mod3; (mod1—no ISD, mod2—mouse native loop/no ISD, mod3—sheep loop/no ISD).

Conclusions: These data show that further optimization of the snRNA ISD and loop boosts SCN1A poison exon 20N exclusion in HepG2 cells.

Exon 20N Skipping Activity of U7 snRNA Constructs in Dravet Syndrome Neurons

The two best antisense U7 snRNA sequences based on exon 20N skipping activity in vitro were selected and packaged into a self-complementary AAV9 (scAAV9) genome followed by AAV production. This snRNA packaging strategy, which minimizes the repetitive sequences within the genome, lead to high titer, full-length self-complementary AAV preparations with the intended genome (containing 2×snRNAs) as confirmed by long-read sequencing (data not shown). The scAAV9 constructs targeting poison exon were delivered to SCN1+/−GABAergic inhibitory neurons (in vitro) and to P0 neonate BL6 wildtype mice (in vivo) with intracerebroventricular (ICV) injections. In vitro AAV9 null (empty capsid) and untreated (UNT) cells were used as negative controls.

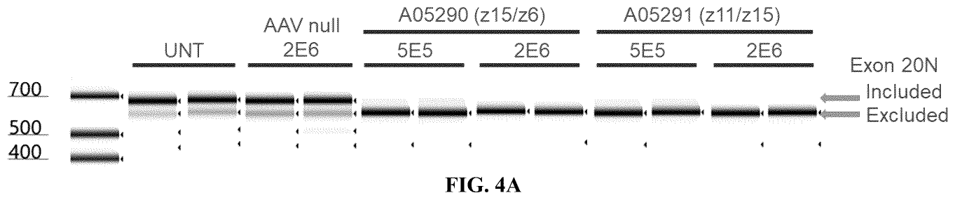

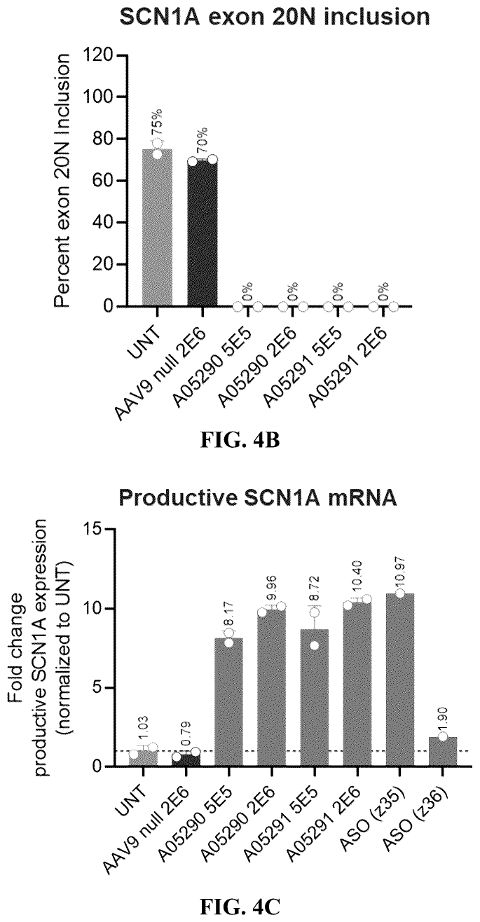

FIG. 4A shows a tapestation image of the RT-PCR products for exon 20N skipping in SCN1A+/−GABAergic neurons and cells after U7 snRNA treatments using increasing MOIs of AAV9 (dual U7 snRNA cassettes expressing fusion spacers under mouse U7 and mouse U1 promoters); A05290 (U7 Fusion z15+z6) and A05291 (U7 Fusion z11+z15).

Data are quantified in FIGS. 4B and 4C. There was complete elimination of SCN1A poison exon 20N with U7 snRNAs and a 10-fold increase in productive SCN1A RNA in SCN1A+/−GABAergic neurons 7 days post AAV9 transduction (FIG. 4C).

Conclusion: These data show elimination of SCN1A poison exon with 2 lead U7 snRNAs constructs 7 days post-transduction with AAV9 in SCN1A+/−GABAergic neurons.

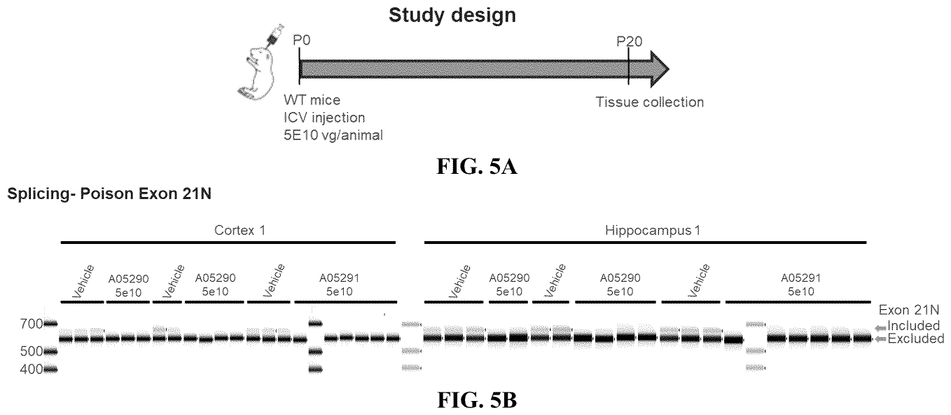

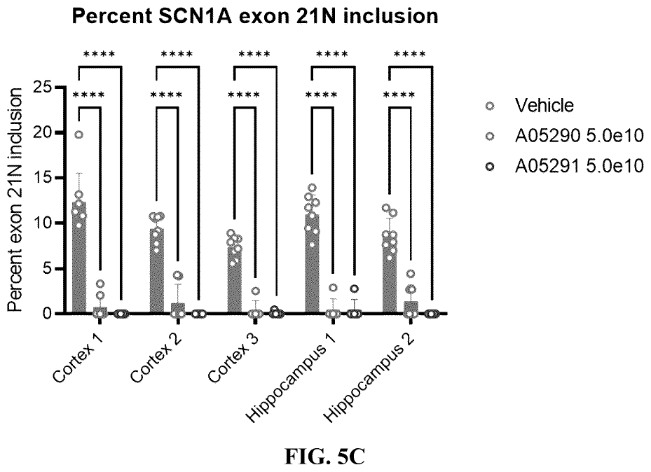

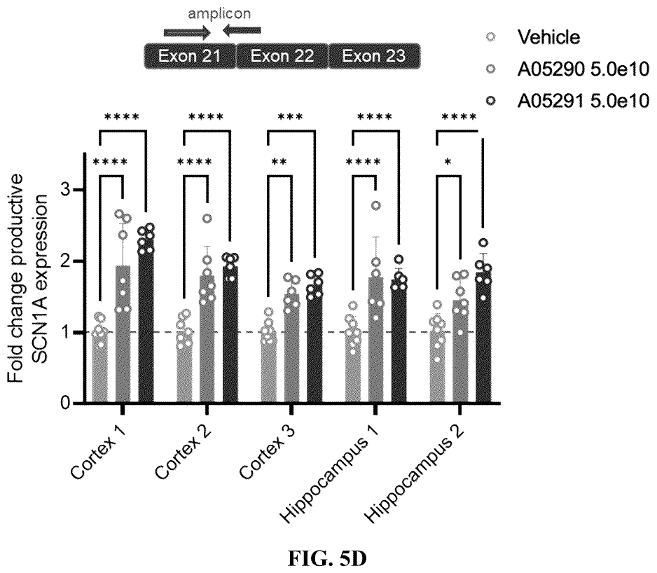

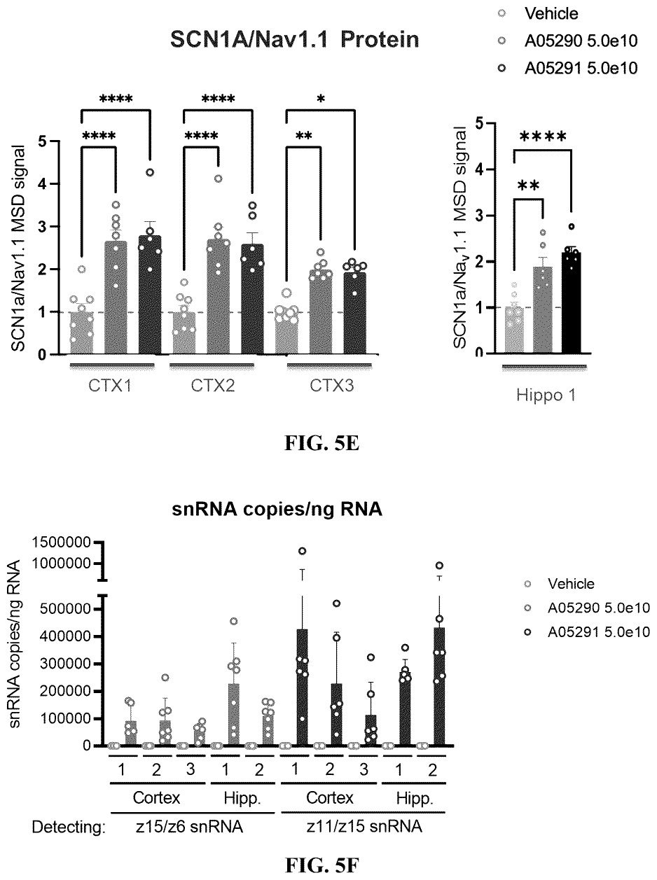

The lead constructs A05290 and A05291 (both containing 2×U7 fusion spacer cassettes) were further evaluated in vivo. The study design is shown in FIG. 5A. FIG. 5B shows a representative tapestation image for cortex and hippocampus samples of the RT-PCR products after U7 snRNA treatments with A05290 and A05291, showing the SCN1A poison exon 21N (mouse equivalent of poison exon 20N in human) non-skipped band, or the skipped product. Vehicle treated samples were used as a negative control. Data are quantified in FIG. 5C from different slices of cortex and hippocampus. These data indicate that scAAV9 delivered U7 snRNAs can promote efficient skipping of SCN1A poison exon in mouse cortex and hippocampus post neonatal ICV delivery.

FIG. 5D shows the levels of productive SCN1A mRNA (without the poison exon) 20 days post ICV injection with AAV9 expressing dual U7 snRNA cassettes of 3 areas of the cortex and 2 areas of the hippocampus. FIG. 5E shows SCN1A/Nav1.1 protein expression levels in cortex and hippocampus 20 days post ICV injection with AAV9 expressing dual U7 snRNA cassettes. FIG. 5F shows snRNA expression in the cortex and hippocampus after AAV9 expressing snRNAs treatment. These data indicate that scAAV9 delivered U7 snRNAs increase SCN1A productive mRNA and protein in mouse cortex and hippocampus post neonatal ICV delivery.

Conclusion: Dose dependent U7 snRNA expression and complete poison exon 20N skipping was observed in the Dravet Syndrome neurons 7 days post treatment. Similarly, complete exon skipping was observed in mouse cortical and hippocampal regions that resulted in ~2.0-fold increase in productive SCN1A mRNA and Nav1.1 protein, 3 weeks post treatment. Overall, these data indicate that AAV-delivered snRNAs can be used to successfully target SCN1A poison exon to rescue haploinsufficiency in vitro and in vivo.

### Example 2: SYNGAP1 Poison Exon 11 Exclusion

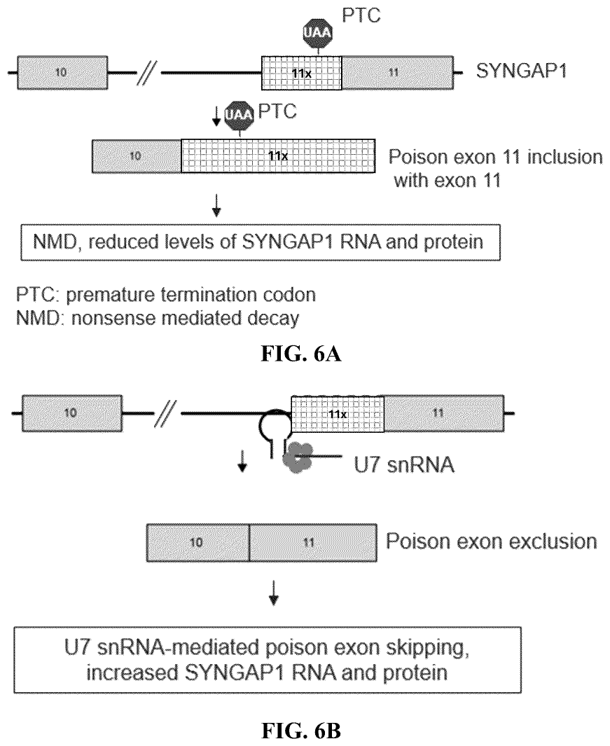

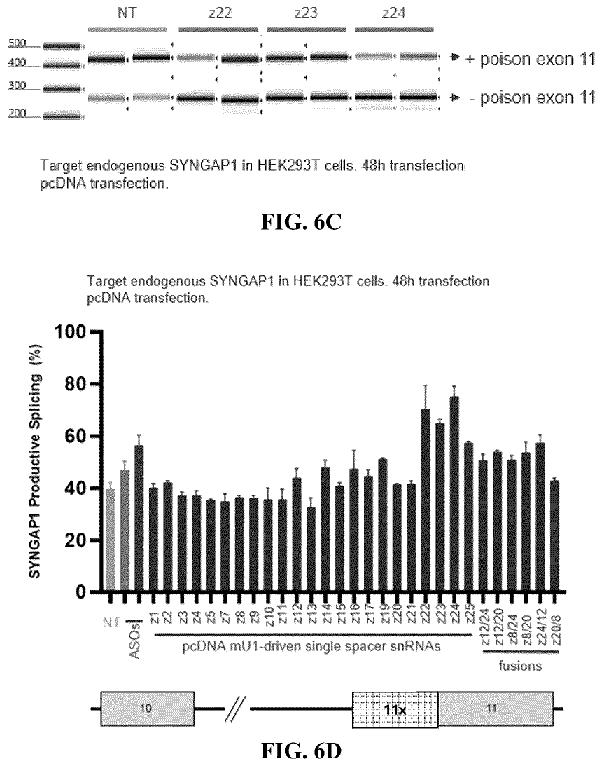

Inclusion of SYNGAP poison exon 11 (shown as a hatched box which contains a premature termination codon (PTC) leads to nonsense mediated decay (NMD) transcript degradation and haploinsufficiency (FIG. 6A). FIG. 6B shows the mechanism of action of SYNGAP poison exon 11 skipping by U7 snRNAs to restore frame and function. U7 snRNAs were engineered to bind splicing regulatory sequences within the SYNGAP1 poison exon 11 to promote skipping and restoration of frame and function. FIG. 6C shows a representative tapestation image of the RT-PCR products after U7 snRNA treatments using single U7 spacers (in pcDNA, z22, z23 and z24), showing the SYNGAP poison exon 11 non-skipped band (containing the poison exon, top fragment), or the skipped product (exclusion of the poison exon, bottom band). Non-targeting U7 (NT), was used as a negative control. ASOs previously shown to promote exclusion of poison exon 11 were used as a positive control. (FIG. 6D) Depicts quantification of tapestation results plotted as percentage of poison exon exclusion post-treatment with multiple single and fusion spacers U7 snRNAs in Hek293-T cells targeting distinct areas of SYNGAP1 pre-mRNA (shown in the graph and diagram below).

Conclusion: U7 snRNAs can binding multiple regions of SYNGAP1 pre-mRNA and promote skipping of poison exon 11 in HEK293T cells.

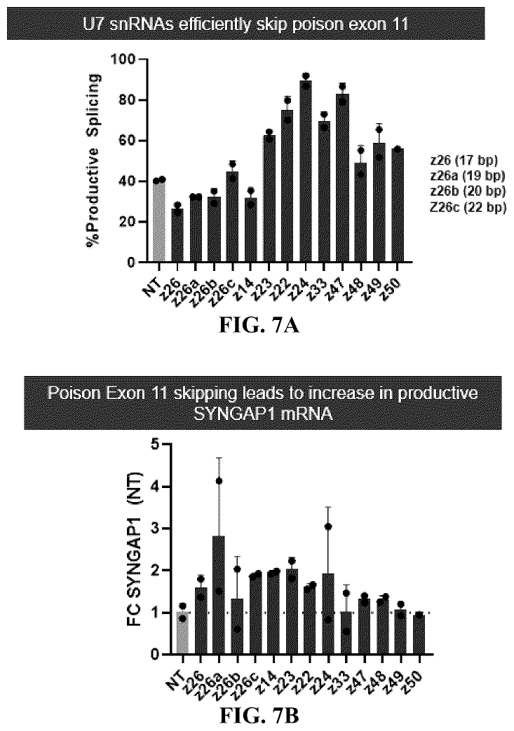

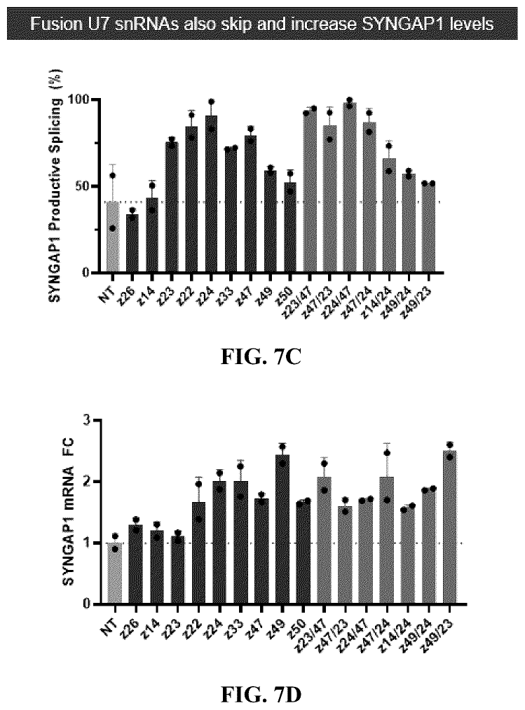

Quantification of percentage of endogenous SYNGAP1 poison exon 11 skipping determined by the splicing assay (FIG. 7A) or expression of productive SYNGAP1 mRNA determined by qRT-PCR (FIG. 7B) post-treatment with single synthetic U7 snRNAs was performed in HEK293-T cells. Non-targeting U7 (NT) was used as a negative control. Quantification of percentage of endogenous SYNGAP1 poison exon 11 skipping determined by the splicing assay (FIG. 7C) or expression of productive SYNGAP1 mRNA determined by qRT-PCR (FIG. 7D) post-treatment with new optimized single and fusion U7 snRNAs in HEK293-T cells. Non-targeting U7 (NT) was used as a negative control.

Conclusion: Optimized synthetic U7 snRNAs single and fusion spacers promote efficient SYNGAP1 poison exon 11 skipping and increase the expression of productive SYNGAP1 mRNA in HEK293T cells.

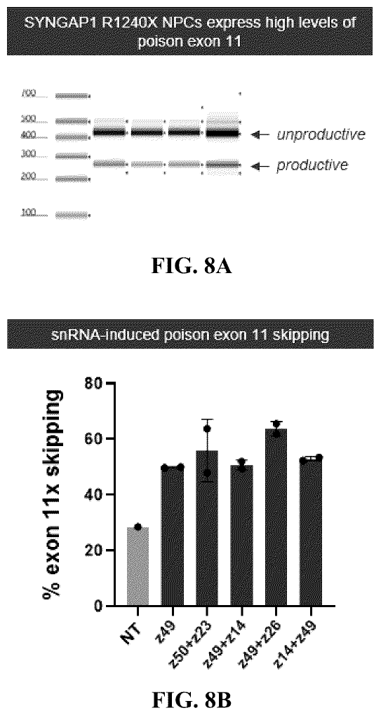

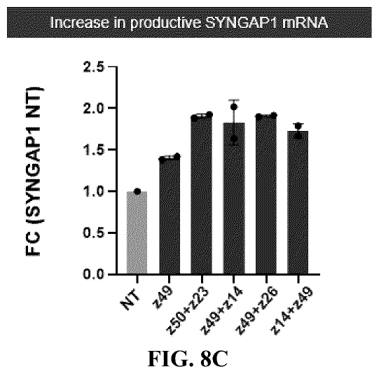

FIG. 8A shows a tapestation image of the RT-PCR products of SYNGAP1 exon 11 levels in patient neural progenitor cells (NPCs) containing the R1240X mutation. Patient NPCS show high levels of SYNGAP1 poison exon 11 inclusion (top band). Quantification of the RT-PCR products for percentage of SYNGAP1 poison exon 11 skipping post-U7 snRNA treatments in patient-derived NPCs (FIG. 8B). Non-targeting U7 (NT) was used as a negative control. FIG. 8C depicts the expression of productive SYNGAP1 mRNA (without the poison exon 11) determined by qRT-PCR following treatment with U7 snRNAs.

Conclusion: Multiple U7 snRNAs promote efficient SYNGAP1 poison exon 11 skipping and increase the expression of productive SYNGAP1 mRNA in patient derived NPCs.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodied herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. An RNA-targeting nucleic acid molecule comprising a small nuclear RNA (snRNA), wherein the snRNA comprises a targeting sequence that binds an SYNGAP1 RNA sequence.

2. The RNA-targeting nucleic acid molecule of claim 1, wherein the SYNGAP1 RNA sequence is Exon 11.

3. The RNA-targeting nucleic acid molecule of claim 1, wherein the SYNGAP1 targeting sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one of SEQ ID NOs: 181-279.

4. The RNA-targeting nucleic acid molecule of claim 1, wherein the snRNA comprises a stem loop (SL).

5. The RNA-targeting nucleic acid molecule of claim 4, wherein the SL comprises one or more nucleic acid sequences set forth in any one of SEQ ID NOs: 84-124.

6. The RNA-targeting nucleic acid molecule of claim 1, wherein the SYNGAP1 RNA sequence is a pre-mRNA or mRNA sequence.

7. The RNA-targeting nucleic acid molecule of claim 1, wherein the snRNA comprises an Sm binding domain (SmBD).

8. The RNA-targeting nucleic acid molecule of claim 7, wherein the SmBD is a U1, U2, U4, or U5 SmBD.

9. The RNA-targeting nucleic acid molecule of claim 7, wherein the SmBD comprises a nucleic acid sequence set forth in any one of SEQ ID NOs: 160 or 161.

10. The RNA-targeting nucleic acid molecule of claim 1, wherein the snRNA comprises a 5′ interaction stabilizer domain (5′ISD).

11. The RNA-targeting nucleic acid molecule of claim 10, wherein the 5′ISD comprises the nucleotide sequence ggagt, cctct, ggaggt, cctcct, agccag, ggaag, gaagaag, gttg, ccgaa, taaggag, gaag, or ggctt.

12. A vector comprising one or more snRNA of claim 1.

13. The vector of claim 14, wherein the vector is an AAV vector.

14. The AAV vector of claim 13, wherein the snRNA is operably linked to a promoter.

15. The AAV vector of claim 13, wherein the snRNA is operably linked to a U7 promoter or a U1 promoter.

16. The AAV vector of claim 13, wherein the snRNA is operably linked to a downstream terminator (DT).

17. The AAV vector of claim 13, wherein the snRNA is operably linked to a U7 downstream terminator or a U1 downstream terminator.

18. The AAV vector of claim 13, wherein the vector comprises at least one, at least two, at least three, at least four, or at least five snRNA.

19. The AAV vector of claim 18, wherein the least one, at least two, at least three, at least four, or at least five snRNA each target the same target RNA sequences.

20. The AAV vector of claim 18, wherein each snRNA is separated by a buffer sequence.

21. The AAV vector of claim 20, wherein the buffer sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one SEQ ID NOs: 151-157.

22. A method of targeting one or more target RNAs of interest and exon-skipping the one or more target RNAs, comprising contacting the snRNA of claim 1 with a cell comprising the one or more target RNAs.

23. A SYNGAP1 RNA-targeting nucleic acid molecule comprising a targeting sequence set forth in any one of SEQ ID NOs: 181-279.

24. A method of treating a disease or disorder in a subject comprising administering to the subject an RNA-targeting nucleic acid molecule of claim 1 or an AAV vector of claim 13.

25. The method of claim 24, wherein the disease or disorder is a genetic epilepsy.

26. The method of claim 24, wherein the disease or disorder is SYNGAP1-related epilepsy.

27. The method of claim 24, wherein the administration is systemic, intravenous, or intracerebroventricular.
